(19) United States
(12) Patent Application Publication
(10) Pub. No.: US 2026/0166051 A1
(43) Pub. Date: Jun. 18, 2026

(54) COMPOUNDS AND METHODS FOR TREATING CANCER

(71) Applicant: The Regents of the University of California, Oakland, CA (US)

(72) Inventors: Michael E. Jung, Los Angeles, CA (US); Frank Pajonk, Los Angeles, CA (US); Ling He, Los Angeles, CA (US); Xiaohong Chen, Los Angeles, CA (US); Hui Ding, Los Angeles, CA (US)

(21) Appl. No.: 19/123,284

(22) PCT Filed: Oct. 23, 2023
(86) PCT No.: PCT/US2023/035704

§ 371 Date: Apr. 22, 2025

Related U.S. Application Data
(60) Provisional application No. 63/418,808, filed on Oct. 24, 2022.

(51) Int. Cl. A61K 31/55; A61K 31/137; A61K 31/166; A61K 31/18; A61K 31/325; A61K 31/341; A61K 31/404; A61K 31/4402; A61K 31/4439; A61K 31/4453; A61K 31/454; A61K 31/4709; A61K 31/496; A61K 31/517; A61K 31/5377; A61P 35/00; C07C 211/27; C07C 211/29; C07C 217/58; C07C 233/65; C07C 271/28; C07C 311/37; C07C 311/47; C07D 207/34; C07D 209/42; C07D 213/38; C07D 215/22; C07D 239/80; C07D 239/96; C07D 295/26; C07D 307/52; C07D 401/12; C07D 403/12; C07D 405/12

(52) U.S. Cl. CPC A61K 31/55; A61K 31/137; A61K 31/166; A61K 31/18; A61K 31/325; A61K 31/341; A61K 31/404; A61K 31/4402; A61K 31/4439; A61K 31/4453; A61K 31/454; A61K 31/4709; A61K 31/496; A61K 31/517; A61K 31/5377; A61P 35/00; C07C 211/27; C07C 211/29; C07C 217/58; C07C 233/65; C07C 271/28; C07C 311/37; C07C 311/47; C07D 207/34; C07D 209/42; C07D 213/38; C07D 215/22; C07D 239/80; C07D 239/96; C07D 295/26; C07D 307/52; C07D 401/12; C07D 403/12; C07D 405/12

(57) ABSTRACT

The present disclosure relates to compounds for treating cancer. The compounds may prevent conversion of non-stem cancer cells into cancer-initiating cells caused by radiation treatment.

### CROSS-REFERENCE TO RELATED APPLICATIONS

This application claims the benefit of priority to U.S. Provisional Patent Application Ser. No. 63/418,808, filed on Oct. 24, 2022, the contents of which are hereby incorporated by reference in its entirety.

### GOVERNMENT SUPPORT

This invention was made with government support under CA200234 and CA211015, awarded by the National Institutes of Health. The government has certain rights in the invention.

### BACKGROUND

Solid cancers are thought to be organized hierarchically with a small number of cancer-initiating cells (CICs; often also called “cancer stem cells’) at the apex of this hierarchy, able to repopulate and regrow the tumor, while their progeny lack these traits. It has been reported that breast-cancer initiating cells (BCICs) are relatively resistant to radiation. The radioresistance of BCICs has been independently confirmed by others and similar results have been reported for glioblastoma. This treatment resistance leads to a selective killing of non-stem cancer cells and thereby enrichment for CICs. However, selective killing of non-stem cancer cells alone cannot explain the magnitude of CIC enrichment by radiation. Indeed, it has been demonstrated that radiation not only spares CICs but also triggers a phenotype conversion of non-stem cancer cells into induced CICs (iCICs). Therefore new treatments for cancer are needed.

### SUMMARY OF THE INVENTION

In certain embodiments, the present disclosure provides compounds represented by formula (I) or a pharmaceutically acceptable salt thereof:

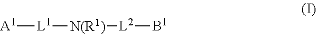

wherein
   (1) L¹ is C₁-C₆ alkylene, C₂-C₆ alkenylene, or —C(O)—;
      R¹ is H, or C₁-C₆ alkylene; and
      A¹ is

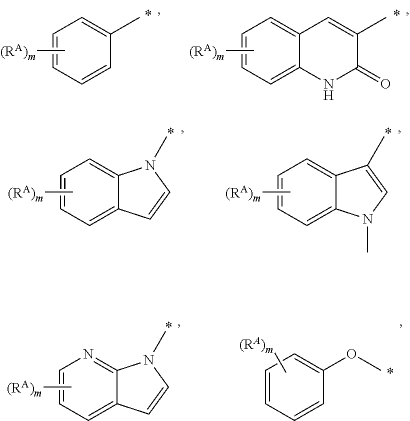

or 5- or 6-membered heteroaryl optionally substituted with one or more R^{A}, where * denotes the linkage to L¹;
or
(2) L¹ is —C(═CH₂)—, —C(R³)(R⁴)—, or —C(O)—; and
   R¹ and A¹ together with the nitrogen to which R¹ and L¹ is attached combine to form:

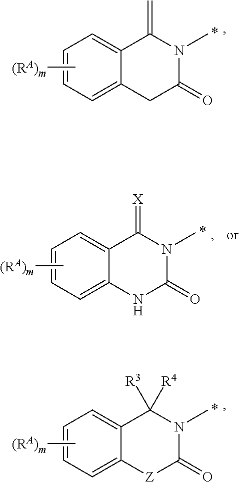

where * denotes the linkage to L²,
   R³ and R⁴ are each independently H, —OH, C₁-C₆ alkyl, C₁-C₆ haloalkyl, or phenyl;
   X is O or CH₂, and
   Z is NH or CH₂;
each R^{A} is independently halo, —OH, —S(O)₂NR⁵R⁶, C₁-C₆ alkyl optionally substituted with amino, C₁-C₆ alkoxy, C₁-C₆ haloalkyl, —NR⁵R⁶, nitro, cyano, or —C(O)NR⁵R⁶;
L² is:
   (1) C₁-C₆ alkylene, C₂-C₆ alkenylene, or —C(O)NH—, or
   (2) L² is **=C(H)—* wherein * denote the linkage to A¹ and ** denotes the attachment to N and R¹ is a bond to L²;
B¹ is aryl optionally substituted with one or more R^{B} groups, 5- or 6-membered heteroaryl optionally substituted with one or more R^{B} groups, or NR⁵R⁶;
each R^{B} is independently halo, C₁-C₆ alkoxy, —C(O)C₁-C₆ alkyl, —S(O)₂NR⁵R⁶, or —C(O)NR⁵R⁶;
R⁵ and R⁶ are each independently H, alkyl, cycloalkyl, heterocyclyl, or phenyl, wherein alkyl, cycloalkyl, heterocyclyl, and phenyl is optionally substituted with R⁷, or
   R⁵ and R⁶ taken together with the attached nitrogen form a heterocyclyl optionally substituted with R⁷;
R⁷ is —OH, halo, C₁-C₆ alkyl, C₁-C₆ alkylene-phenyl, or phenyl optionally substituted with halo;
m is 0, 1, 2, or 3; and
n is 0, 1, 2, or 3.

In certain embodiments, the present disclosure provides a method of treating or cancer, comprising administering to the subject a compound of the present disclosure.

Numerous embodiments are further provided that can be applied to any aspect of the present invention described herein.

### BRIEF DESCRIPTION OF THE DRAWINGS

FIG. 1A-IB. FIG. 1A. Compound 2 inhibits self-renewal in patient-derived glioma cells.

FIG. 1B. Compound 2 penetrates the BBB in mice.

FIG. 2. MCX017 and MCX079 prevent spontaneous and radiation-induced phenotype conversion of non-stem glioma cells into glioblastoma-initiating cells.

FIG. 3. Whole kinome screening identifies MCX017 as a multikinase inhibitor with activity against FLT3, TNIK and JNK3.

FIG. 4. Western blotting shows down-regulation of p-ERK and p-AKT (FLT3 targets) and stabilization of cytosolic beta-catenin (TNIK target) in response to treatment of patient-derived glioblastoma cells with MCX017 or MCX079.

FIGS. 5A-D. Toxicity of the analogs MXC017, 079 and 021 in fibroblasts (NIH3T3, FIG. 5A), microglia cells (EOC20, FIG. 5B), human astrocytes (FIG. 5C) or murine neural stem/progenitor cells (FIG. 5D).

FIGS. 6A and 6B. Sphere-forming assay data from compounds according to the present disclosure.

FIG. 7. Sphere-forming assay data from compounds according to the present disclosure.

FIG. 8. Sphere-forming assay data according compounds according to the present disclosure.

FIG. 9. Sphere-forming assay data from compounds according to the present disclosure.

FIG. 10. Sphere-forming assay data from compounds according to the present disclosure.

FIG. 11. Sphere-forming assay data from compounds according to the present disclosure.

FIG. 12. Sphere-forming assay data from compounds according to the present disclosure.

FIG. 13. Sphere-forming assay data from compounds according to the present disclosure.

FIG. 14. Sphere-forming assay data from compounds according to the present disclosure.

FIG. 15. Sphere-forming assay data from compounds according to the present disclosure.

FIG. 16. Sphere-forming assay data from compounds according to the present disclosure.

FIG. 17. Sphere-forming assay data from compounds according to the present disclosure.

FIG. 18. Sphere-forming assay data from compounds according to the present disclosure.

FIGS. 19A and 19B. Sphere-forming assay data from compounds according to the present disclosure.

FIG. 20. Sphere-forming assay data from compounds according to the present disclosure.

FIG. 21. RNASeq from assays performed in the presence of compounds according to the present disclosure.

FIG. 22. RNASeq from assays performed in the presence of compounds according to the present disclosure.

FIG. 23. Western blots of HIF1a and HIF2a from assays performed in the presence of compounds according to the present disclosure.

FIG. 24. Western blots of HIF1a and HIF2a from assays performed in the presence of compounds according to the present disclosure.

FIG. 25. Western blots of HIF1a and HIF2a from assays performed in the presence of compounds according to the present disclosure.

FIG. 26. Sphere-forming assay data from compounds according to the present disclosure.

FIG. 27. Reprogramming assay data assessed by flow cytometry of compounds according to the present disclosure.

FIG. 28. Plating efficiency assay data from compounds according to the present disclosure.

FIGS. 29A and 29B. Plating efficiency assay data from compounds according to the present disclosure.

FIGS. 30A and 30B. Plating efficiency assay data from compounds according to the present disclosure.

FIGS. 31A and 31B. Plating efficiency assay data from compounds according to the present disclosure.

FIG. 32. Plating efficiency assay data from compounds according to the present disclosure.

FIG. 33. Sphere-forming assay data from compounds according to the present disclosure.

FIG. 34. Sphere-forming assay data from compounds according to the present disclosure.

FIG. 35. Sphere-forming assay data from compounds according to the present disclosure.

FIG. 36. Sphere-forming assay data from compounds according to the present disclosure.

FIG. 37. Sphere-forming assay data from compounds according to the present disclosure.

FIGS. 38A and 38B. PK studies for MXC-017 and MXC-079 Mice were treated with a single dose of the novel compounds at 50 mg/kg. Plasma and brain samples were taken at indicated time points and compound concentration measured by mass spectrometry. MXC-017 showed a 455% enrichment in brain tissue compared to 263% for MXC-079.

FIGS. 39A-D. FIG. 39A NSG mice were injected with HK374 cells. After 2 weeks post grafting, mice received a single dose of 10 Gy and 5 daily injections/week of MXC-017 or saline for 2 weeks. Tumors were harvested and an in vitro extreme limiting dilution assay was performed. FIG. 39B MXC-017 was well tolerated by NSG mice. (FIG. 39C) MXC-017 significantly reduced the number of GSCs. Combination of radiation and MXC-017 in vivo eliminated all GSCs. FIG. 39D Elimination of GSCs was reflected by the complete loss of cells with self-renewing capacity.

FIGS. 40A-B MXC-017 prolongs survival in PDOX models of GBM without normal tissue toxicity (FIG. 40A) A comparison of sections of kidneys, lung, spleen, heart, or liver from saline-treated mice (top) and mice treated with MXC-017 for 2 weeks (5 days on, 2 days off) at 50 mg/kg (bottom) did not reveal any toxicity of MXC-017. (FIG. 40B) Treat of PDOX-bearing with a single dose of 10 Gy and daily injection with MXC-017 (5 days on, 2 days off; 50 mg/kg) significantly prolonged median survival from 29 to 60 days (p<0.0001, log-rank test).

FIGS. 41A-B. Immunofluorescent staining of brains of glioma bearing mice using antibodies against Olig2 and Nestin. Combined treatment with radiation and MXC-017 eliminated Nestin-positive glioma stem cells in the tumors while not effecting the sensitive normal tissue cell population of Olig2-positive oligodendrocytes in the brain. Nuclei were counterstained.

FIG. 42. Inhibition of self-renewal of HK-374 glioma stem cells in the absence of radiation. While MHD001, 002, 004, 005, 008 and 009 significantly inhibited self-renewal of unirradiated cells to some degree (69-52%), MHD003 reduced self renewal to 14% (n=3; p<0.0001, one-way ANOVA).

FIG. 43. Inhibition of self-renewal of HK-374 glioma stem cells in combination with 4 Gy. Out of the 10 MHD compounds, only MHD003 significantly reduced self renewal of glioma stem cells to 6% of irradiated controls (DMSO; n=3; p<0.0001, one-way ANOVA).

### DETAILED DESCRIPTION OF THE INVENTION

The present invention is based, at least in part, on the development of new chemical tools and therapeutics that target radiation-induced phenotype conversion. To this end, a series of compounds was designed and synthesized. A combination of high-throughput screening, cell biology, chemical biology, and structural biology was used to identify and validate small molecules that inhibit the conversion of non-stem cancer cells into induced cancer initiating cells (iCICs). Compounds that block the transition from non-stem cancer cells to iCICs were developed. These compounds prevented radiation-induced phenotype conversion of non-stem glioblastoma cells into glioma-initiating cells (GICs).

In certain embodiments, the present disclosure provides formula (I) or a pharmaceutically acceptable salt thereof:

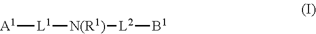

wherein
   (1) L¹ is C₁-C₆ alkylene, C₂-C₆ alkenylene, or —C(O)—;
      R¹ is H; and
      A¹ is

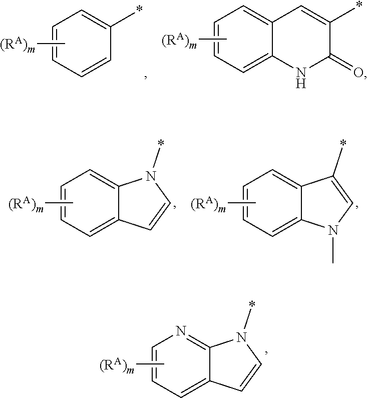

or 5- or 6-membered heteroaryl optionally substituted with one or more R^{A}, where * denotes the linkage to L¹;
or
(2) L¹ is —C(═CH₂)—, —C(R³)(R⁴)—, or —C(O)—; and
   R¹ and A¹ together with the nitrogen to which R¹ and L¹ is attached combine to form:

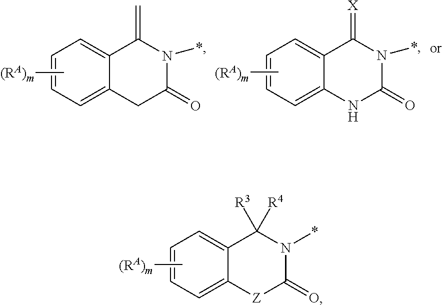

where * denotes the linkage to L²,
   R³ and R⁴ are each independently H, —OH, C₁-C₆ alkyl, C₁-C₆ haloalkyl, or phenyl;
   X is O or CH₂, and
   Z is NH or CH₂;
each R^{A} is independently halo, —OH, —S(O)₂NR⁵R⁶, C₁-C₆ alkyl optionally substituted with amino, C₁-C₆ alkoxy, C₁-C₆ haloalkyl, —NR⁵R⁶, or nitro;
L² is:
   (1) C₁-C₆ alkylene, C₂-C₆ alkenylene, or —C(O)NH—, or
   (2) L² is **=C(H)—* wherein * denote the linkage to A¹ and ** denotes the attachment to N and R¹ is a bond to L²;
B¹ is aryl optionally substituted with one or more R^{B} groups, 5- or 6-membered heteroaryl optionally substituted with one or more R^{B} groups, or NR⁵R⁶;
each R^{B} is independently halo, C₁-C₆ alkoxy, —C(O)C₁-C₆ alkyl, —S(O)₂NR⁵R⁶, or —C(O)NR⁵R⁶;
R⁵ and R⁶ are each independently H, alkyl, cycloalkyl, heterocyclyl, or phenyl, wherein alkyl, cycloalkyl, heterocyclyl, and phenyl is optionally substituted with R⁷, or
   R⁵ and R⁶ taken together with the attached nitrogen form a heterocyclyl optionally substituted with R⁷;
R⁷ is —OH, halo, C₁-C₆ alkyl, C₁-C₆ alkylene-phenyl, or phenyl optionally substituted with halo;
m is 0, 1, 2, or 3; and
n is 0, 1, 2, or 3.

In certain embodiments, the compound is represented by formula (Ia), (Ib), or (Ic):

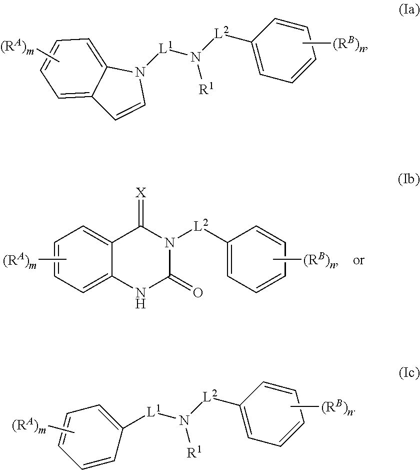

In certain embodiments, the compound is represented by formula (Ia-1), (1b-1) or (1c-1):

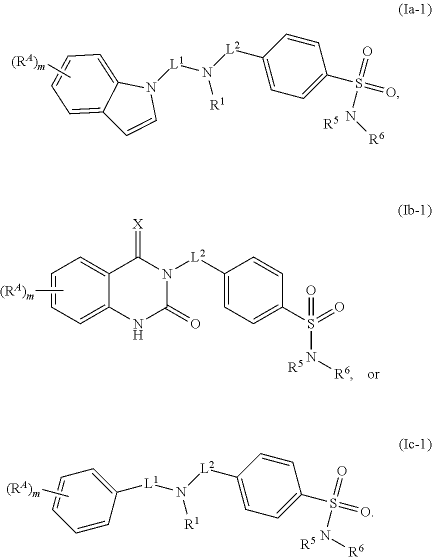

In certain embodiments, the compound is represented by formula (Ia-2), (1b-2), or (Ic-2):

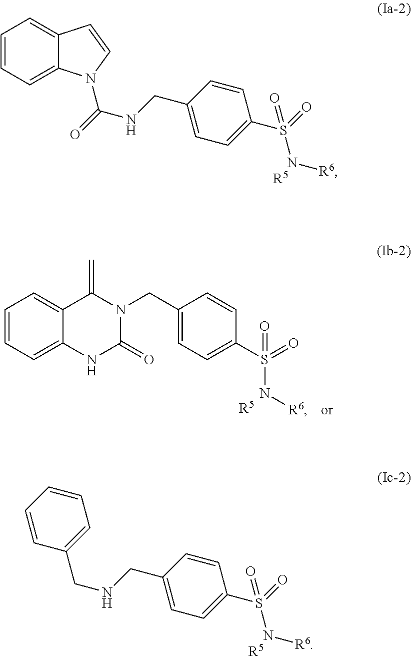

In certain embodiments, the present disclosure provides formula (I) or a pharmaceutically acceptable salt thereof:

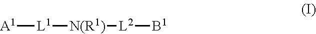

wherein
   (3) L¹ is C₁-C₆ alkylene, C₂-C₆ alkenylene, or —C(O)—;
      R¹ is H, or C₁-C₆ alkylene; and
      A¹ is

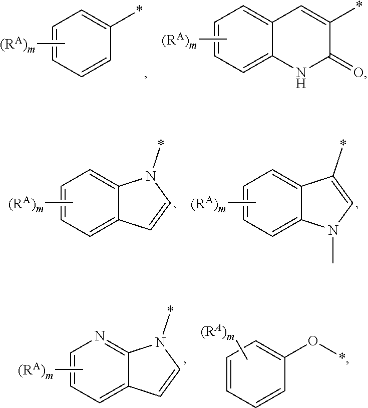

or 5- or 6-membered heteroaryl optionally substituted with one or more R^{A}, where * denotes the linkage to L¹;
or
(4) L¹ is —C(═CH₂)—, —C(R³)(R⁴)—, or —C(O)—; and
   R¹ and A¹ together with the nitrogen to which R¹ and L¹ is attached combine to form:

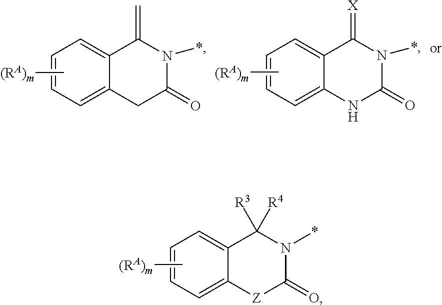

where * denotes the linkage to L²,
   R³ and R⁴ are each independently H, —OH, C₁-C₆ alkyl, C₁-C₆ haloalkyl, or phenyl;
   X is O or CH₂, and
   Z is NH or CH₂;
each R^{A} is independently halo, —OH, —S(O)₂NR⁵R⁶, C₁-C₆ alkyl optionally substituted with amino, C₁-C₆ alkoxy, C₁-C₆ haloalkyl, —NR⁵R⁶, nitro, cyano, or —C(O)NR⁵R⁶;
L² is:
   (1) C₁-C₆ alkylene, C₂-C₆ alkenylene, or —C(O)NH—, or
   (2) L² is **=C(H)—* wherein * denote the linkage to A¹ and ** denotes the attachment to N and R¹ is a bond to L²;
B¹ is aryl optionally substituted with one or more R^{B} groups, 5- or 6-membered heteroaryl optionally substituted with one or more R^{B} groups, or NR⁵R⁶;
each R^{B} is independently halo, C₁-C₆ alkoxy, —C(O)C₁-C₆ alkyl, —S(O)₂NR⁵R⁶, or —C(O)NR⁵R⁶;
R⁵ and R⁶ are each independently H, alkyl, cycloalkyl, heterocyclyl, or phenyl, wherein alkyl, cycloalkyl, heterocyclyl, and phenyl is optionally substituted with R⁷, or
   R⁵ and R⁶ taken together with the attached nitrogen form a heterocyclyl optionally substituted with R⁷;
R⁷ is —OH, halo, C₁-C₆ alkyl, C₁-C₆ alkylene-phenyl, or phenyl optionally substituted with halo;
m is 0, 1, 2, or 3; and
n is 0, 1, 2, or 3.

In certain embodiments, the compound is represented by formula (Ia), (Ib), or (Ic):

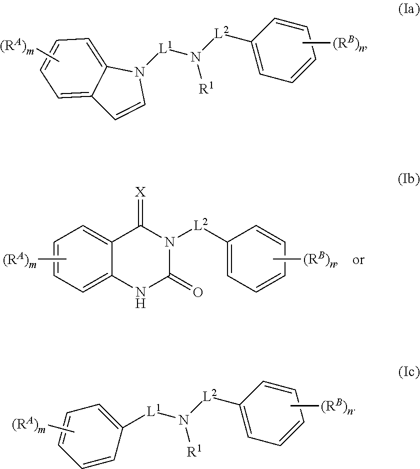

In certain embodiments, the compound is represented by formula (Ia-1), (1b-1) or (1c-1):

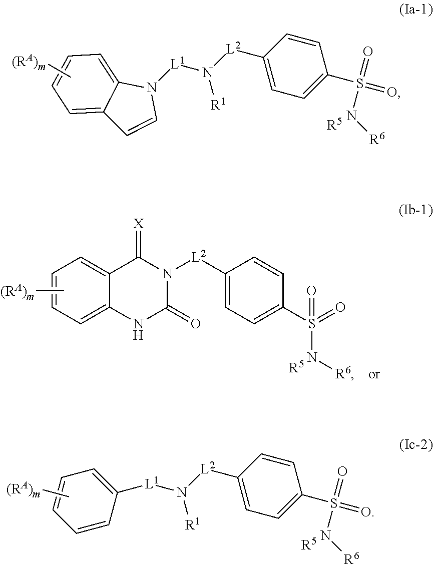

In certain embodiments, the compound is represented by formula (Ia-2), (1b-2), or (Ic-2):

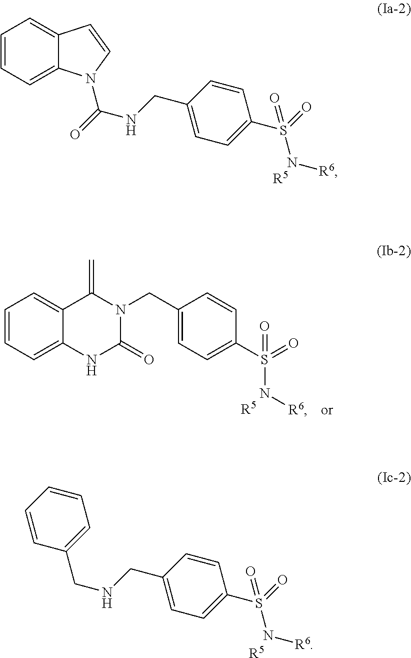

In certain embodiments, the compound is represented by formula (1a-1):

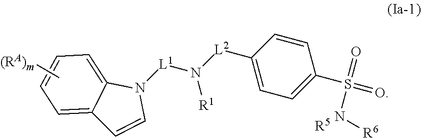

In certain embodiments, the compound is selected from:

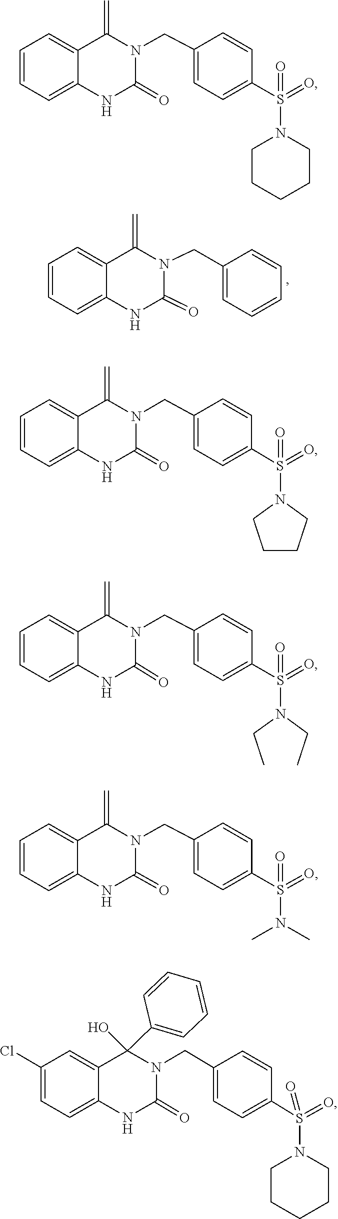 [SMILES: C=C1c2ccccc2NC(=O)N1Cc1ccc(S(=O)(=O)N2CCCCC2)cc1 ; C=C1c2ccccc2NC(=O)N1Cc1ccccc1 ; C=C1c2ccccc2NC(=O)N1Cc1ccc(S(=O)(=O)N2CCCC2)cc1 ; C=C1c2ccccc2NC(=O)N1Cc1ccc(S(=O)(=O)N(CC)CC)cc1 ; C=C1c2ccccc2NC(=O)N1Cc1ccc(S(=O)(=O)N(C)C)cc1 ; O=C1Nc2ccc(Cl)cc2C(O)(c2ccccc2)N1Cc1ccc(S(=O)(=O)N2CCCCC2)cc1]

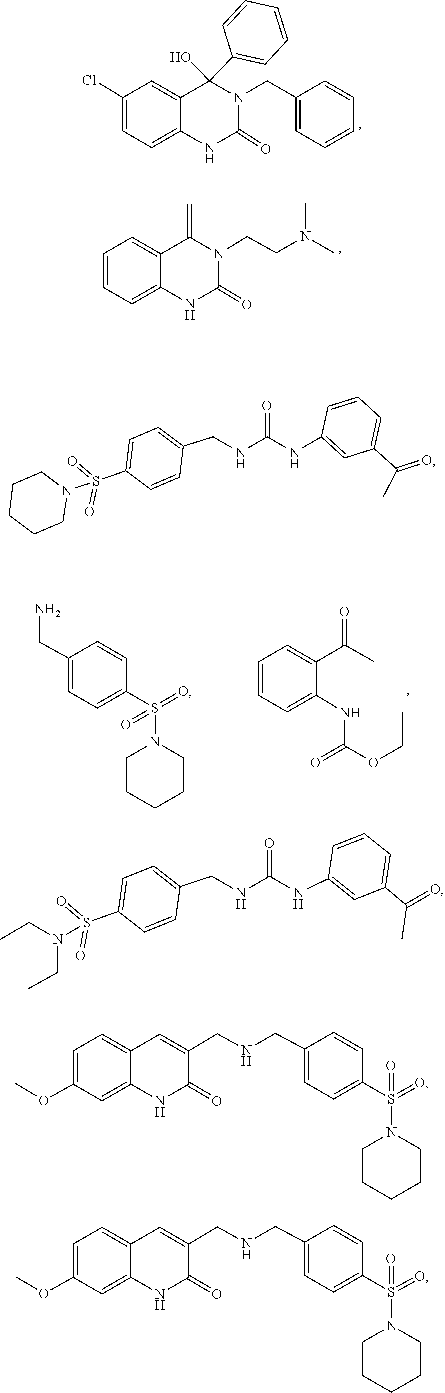

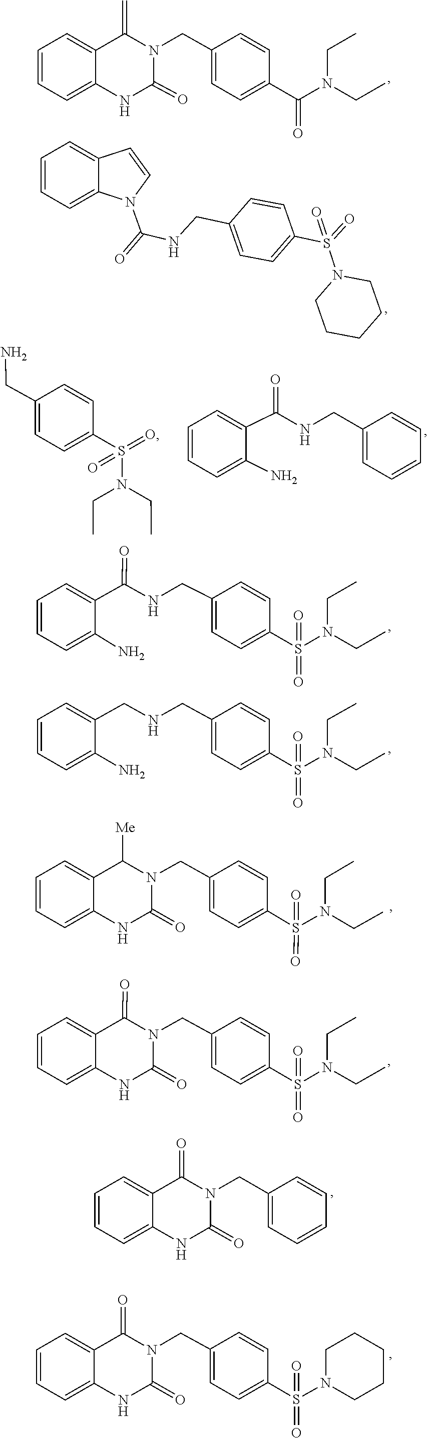

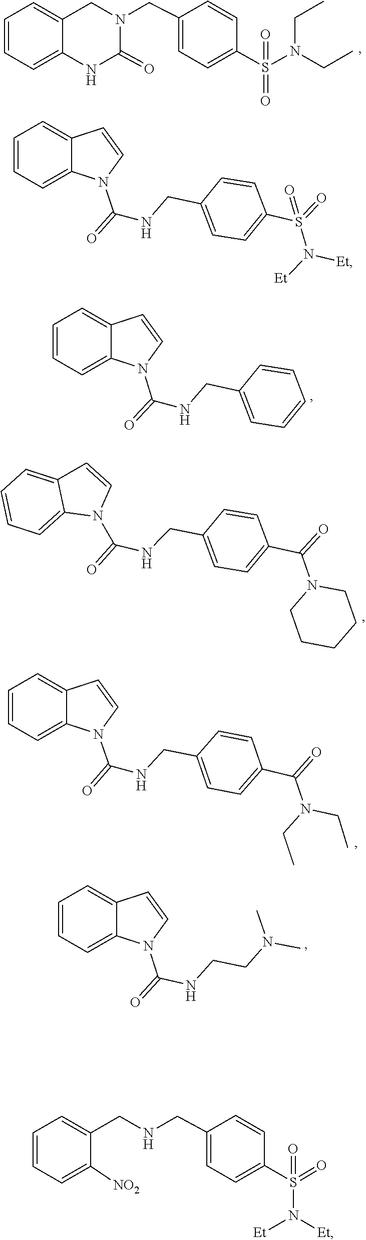

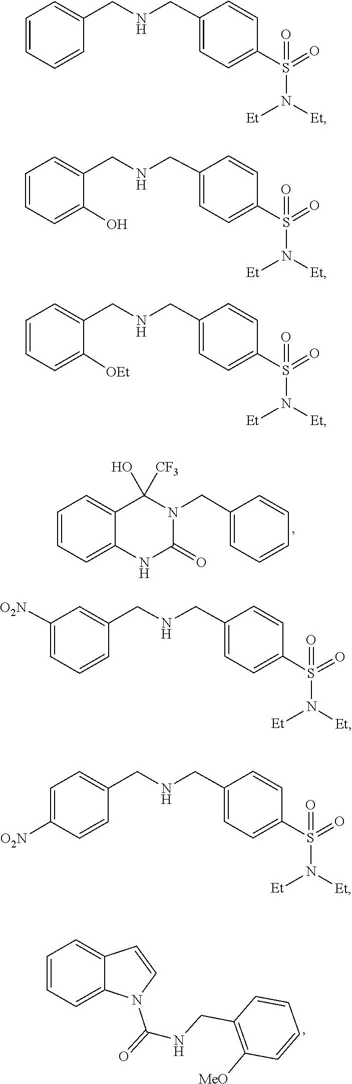

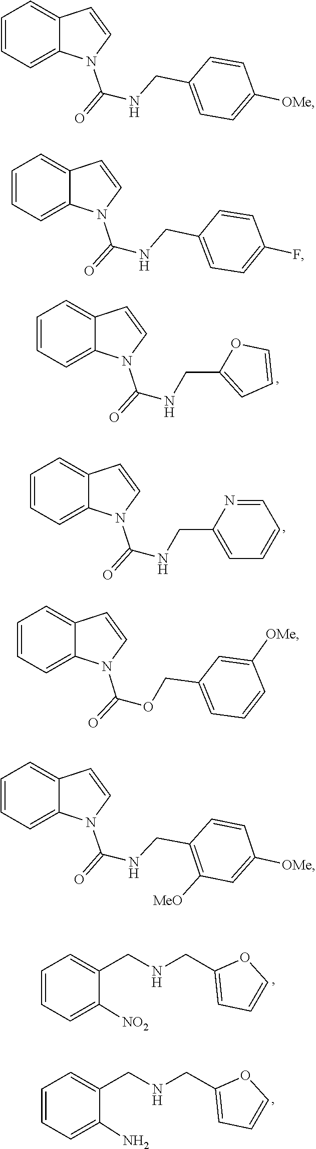

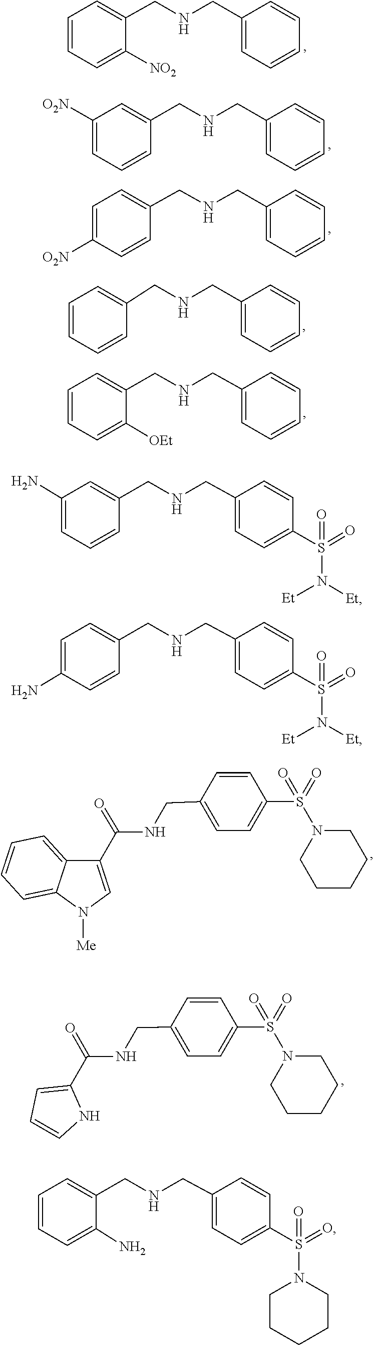

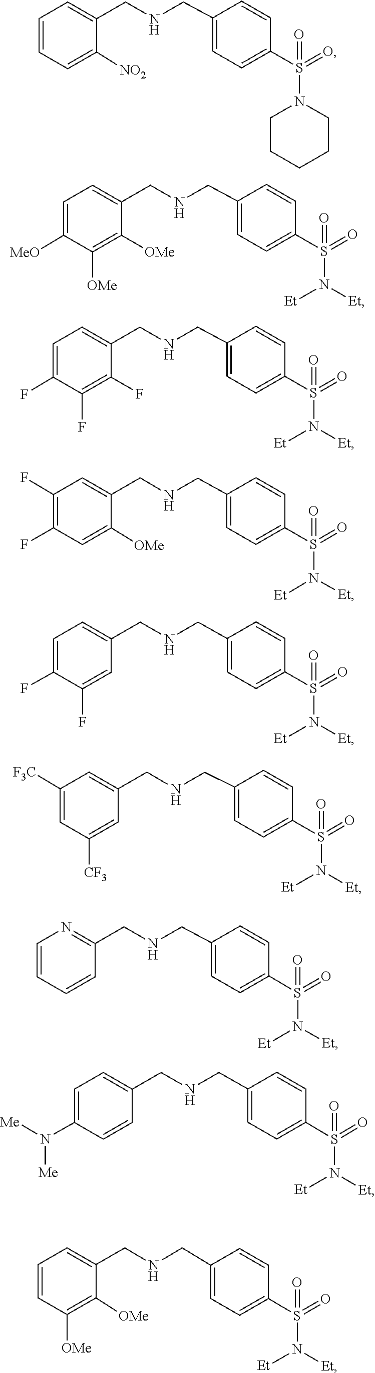

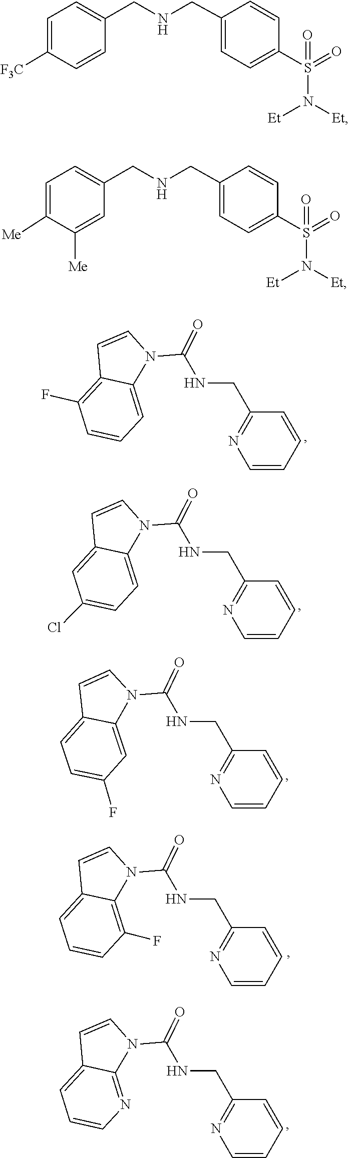

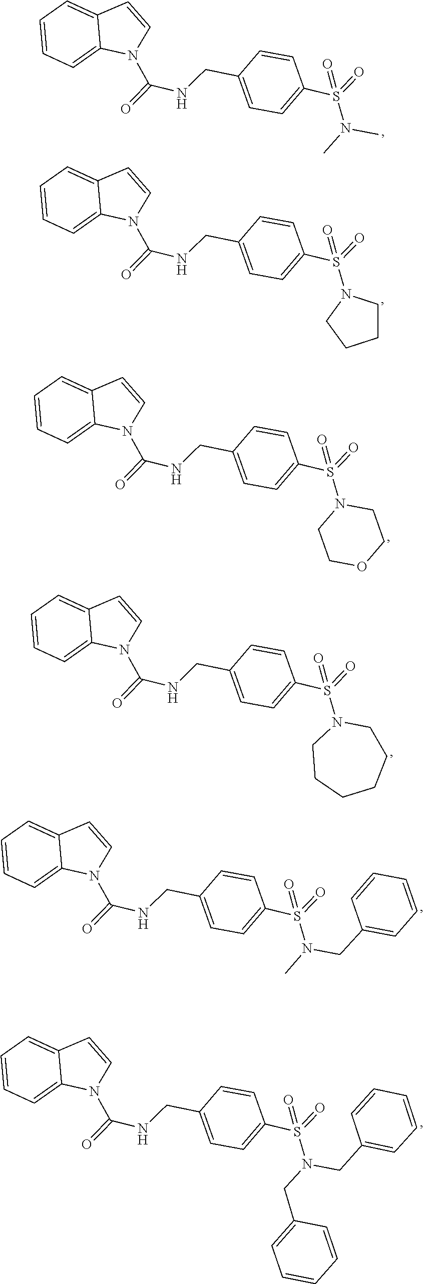 [SMILES: CN(C)S(=O)(=O)c1ccc(CNC(=O)n2ccc3ccccc32)cc1 ; O=C(NCc1ccc(S(=O)(=O)N2CCCC2)cc1)n1ccc2ccccc21 ; O=C(NCc1ccc(S(=O)(=O)N2CCOCC2)cc1)n1ccc2ccccc21 ; O=C(NCc1ccc(S(=O)(=O)N2CCCCCC2)cc1)n1ccc2ccccc21 ; CN(Cc1ccccc1)S(=O)(=O)c1ccc(CNC(=O)n2ccc3ccccc32)cc1 ; O=C(NCc1ccc(S(=O)(=O)N(Cc2ccccc2)Cc2ccccc2)cc1)n1ccc2ccccc21]

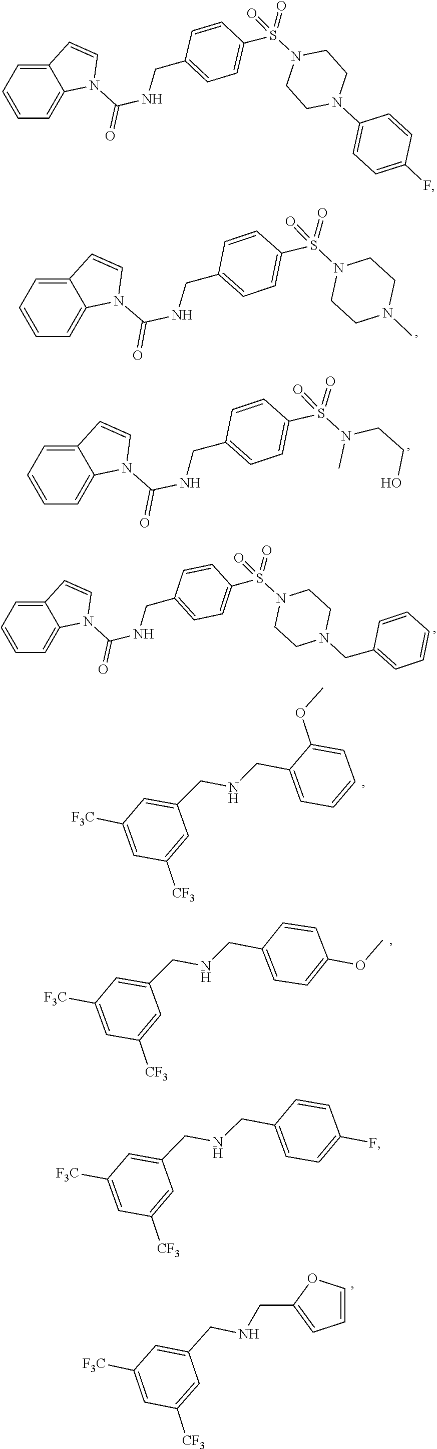

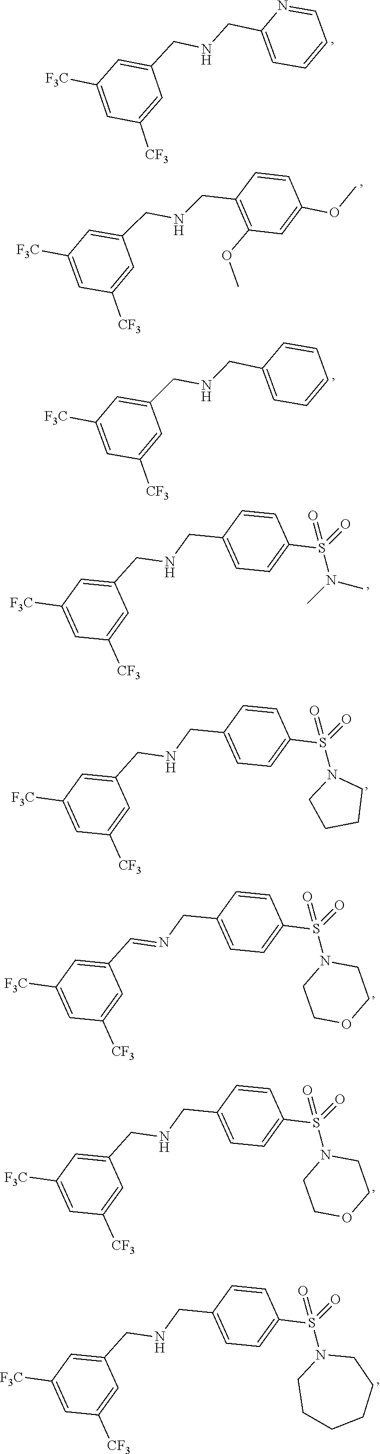

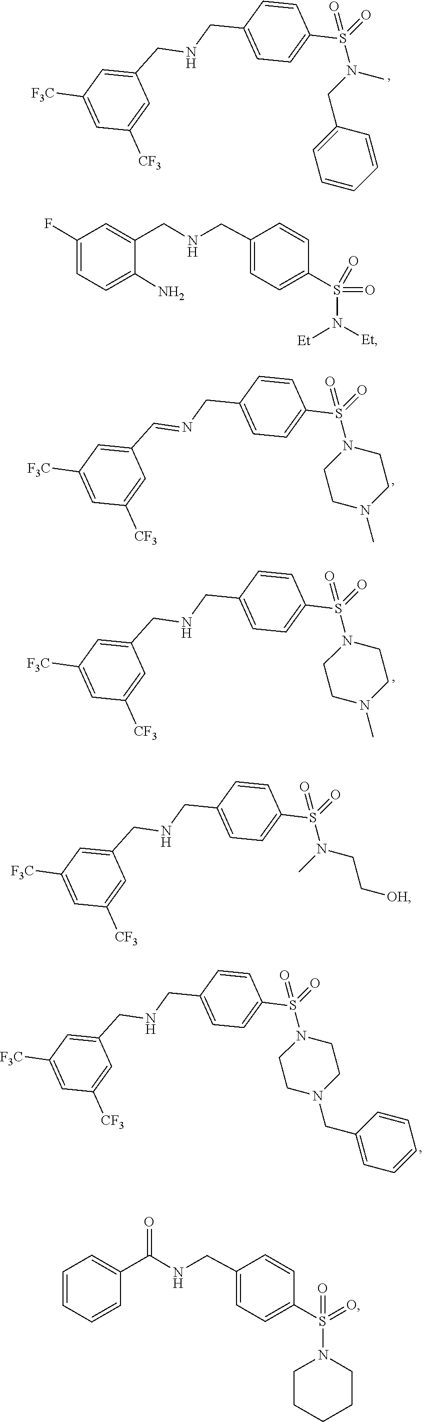

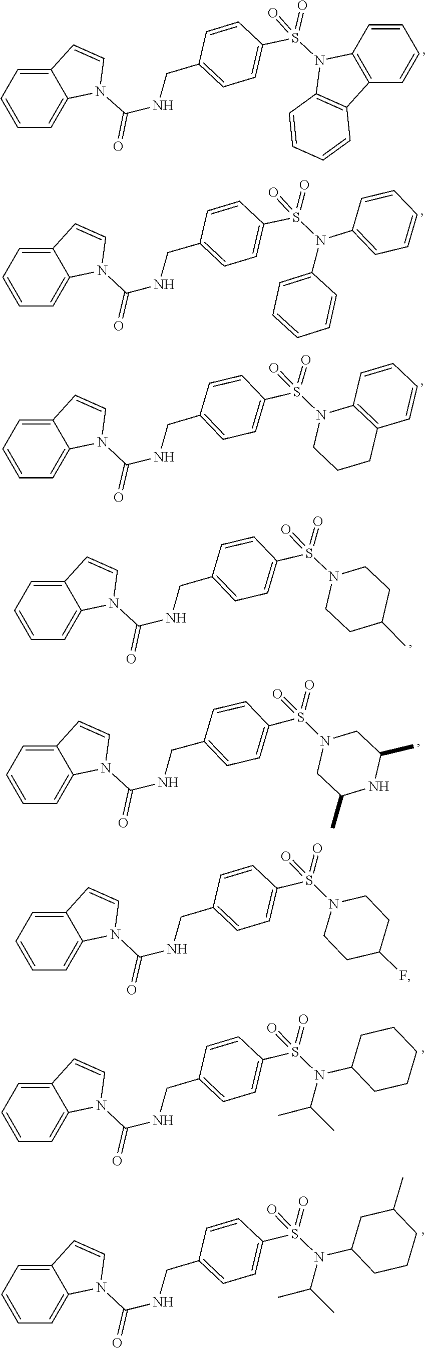

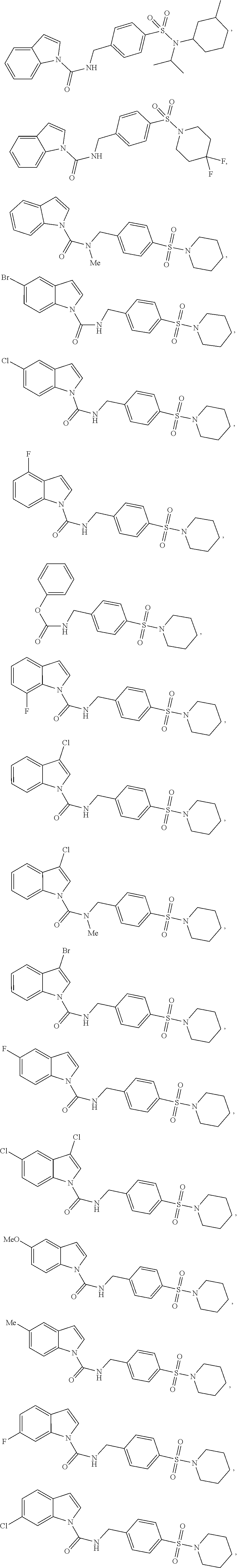

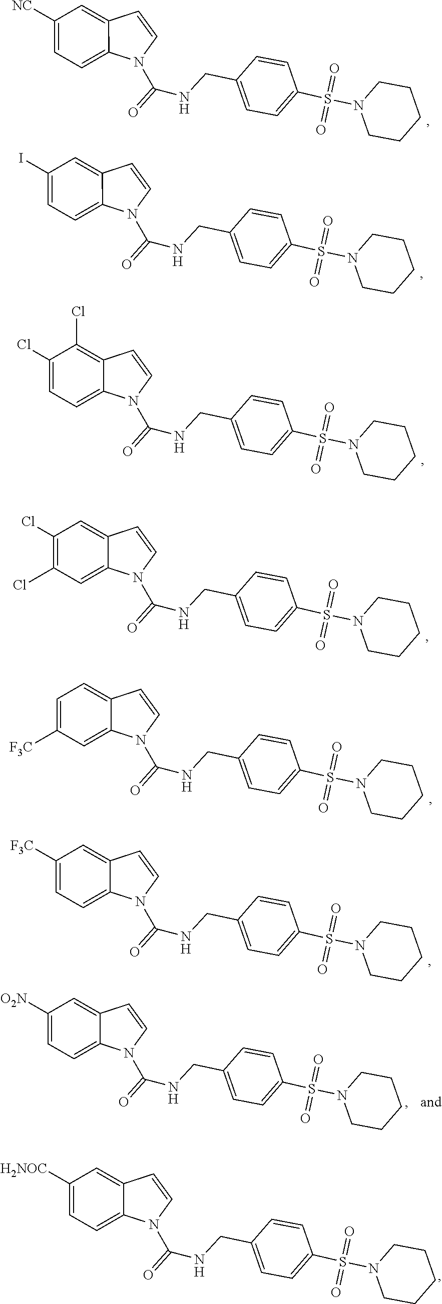

a pharmaceutically acceptable salt thereof.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

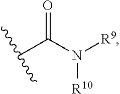

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

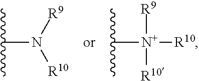

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

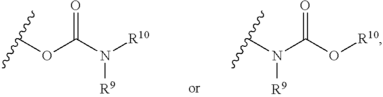

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

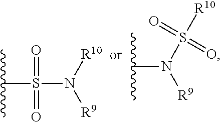

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

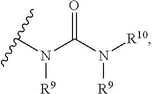

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples, which are included merely for purposes of illustration of certain aspects and embodiments of the present invention, and are not intended to limit the invention.

### High-Throughput Screening

To discover compounds that target radiation-induced phenotype conversion an unbiased, phenotypic screen of 83,000 compounds was performed for their ability inhibit phenotype conversion of non-stem cancer cells into iCICs without significantly increasing the toxicity of radiation. The high-throughput screen made use of the finding that CICs show low 26S proteasome activity that can be exploited to prospectively identify and track CICs when infected with a reporter of proteasome activity. The latter is based on the expression of a fusion protein of the fluorescent protein ZsGreen and the C-terminal degron of murine ornithine carboxylase. In cells with normal proteasome activity the fusion protein is immediately degraded after translation while in cells lacking proteasome activity it accumulates and can be used to identify cells enriched for CICs.

For primary screening of drug libraries ZsGreen-cODC-negative SUM159PT cells (Asterand Bioscience, Cambridge, MA), which show high levels of radiation-induced phenotype conversion (Lagadec, Vlashi et al. 2012) were used as a discovery platform. Cells were sorted by high-speed FACS (FACS Aria I/II) in the UCLA Jonsson Comprehensive Cancer Center (JCCC) and Center for AIDS Research Flow Cytometry Core Facility.

Concurrently, low evaporation 384-well plates were filled by a manifold liquid dispenser (Multidrop 384, Thermo LabSystems) with 35 μL media/well (F-12, hydrocortisone (100 mg/2 mL), 5% FBS, 1% penicillin-streptomycin, 0.1% insulin, and 1M HEPES). Using a liquid handler (Biomek FX, Beckman Coulter, Brea, CA) with custom pin tool (V&P scientific, San Diego, CA), 320 unique compounds (500 nL each from 1 mM stocks in neat DMSO) were then transferred to each 384-well plate according to pre-defined plate maps. The other 64 wells received an equal volume of DMSO alone, serving as negative controls. Following re-suspension of cells in media at a concentration of 200,000 cells/mL, 15 μL cell suspension (3,000 cells) was plated into each well of the pre-filled 384-well plates. After completion of cell plating, 384-well plates were kept at 37° C., 5% CO₂.

Twenty-four hours after plating cells were irradiated with 8 Gy. Five days later, 10 μL Hoechst 33342 solution (25 μg/ml) was added to the cells. The plates were incubated at 37° C., 5% CO₂ for one hour. Plates were scanned using an Acumen Mark III laser-scanning cytometer cytometer (TTP Labtech, Melboum, UK). Laser scanning with a 488 nm laser was used for detection of cells expressing the fusion protein, ZsGreen-cODC. Additionally, scanning with a UC laser at 405 nm allowed for detection Hoechst-stained nuclei, giving a measure of total viable cells.

Z-score statistics were used for hit identification: For every well of each 384-well plate, a z-score

$$( z = \frac{x - μ}{σ} )$$

was calculated for both the ZsGreen-cODC-positive count and the total viable cell count. The population, characterized by the parameters p and a, consists of the 64 negative controls in each plate. Hits for inhibitors of reprogramming are defined as compounds with both 1) a ZsGreen-cODC-positive z-score ≤−2.0 and 2) a total viable cell z-score ≥−1.0. Hits meeting the above criteria from the primary screens were tested in secondary screens. Due to the presence of replicates in the secondary screen setup, the z-score method of identifying hits from the primary screen was not used. Instead, verification of hits in the secondary screen utilized the Student's t-test, in order to take the compound-specific variances into account. A Bonferroni correction for multiple comparisons was used to maintain a false-positive rate of less than 0.05 within each secondary screen.

This screen led to the discovery of 216 primary hits and was narrowed to 169 primary hits with no Lipinski violation (rule of five; predicts the likelihood a compound can be developed into an orally available drug) and CNS Multiparameter Optimization Score of 4 or higher (in silico prediction of BBB penetration).

### Initial Testing

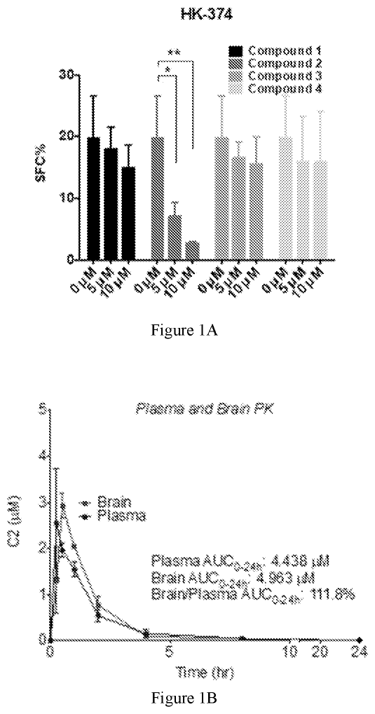

Four compounds were picked out of the list of 169 primary hits and subjected to secondary screening, confirming activity of compound 2 (4-Methylene-3-[4-(1-piperidinylsulfonyl)benzyl]-3,4-dihydro-2(1H)-quinazolinone) (see below) against self-renewal in GBM (FIG. 1A).

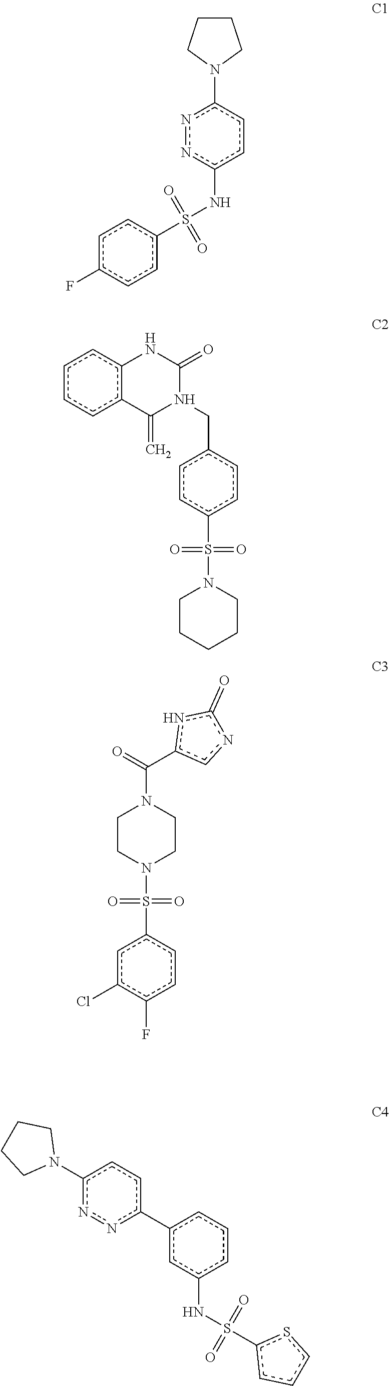

Structure of compound 2 (“C2,” also referred to as MXC001) and the structure of C1, C3, and C4.

In vivo studies confirmed the in silico prediction of BBB penetration by compound 2 (FIG. 1B).

### Synthesis of MXC Compounds:

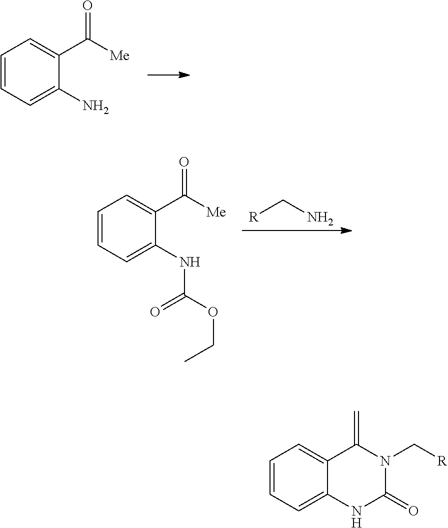

### General Procedure A

A solution of 1.35 g (10 mmol) of 2 ′-aminoacetophenone in 15 mL of dry pyridine was treated with 1.43 mL (15 mmol) of ethyl chioroformate with stirring at 10-15° C. After it had stirred for 1 h, the mixture was concentrated under vacuum. The crude residue was diluted with ethyl acetate (20 mL) and washed with DI water (2×10 mL) and satd. NaCl (15 mL) and dried over Na₂SO₄. It was then filtered and the solvent removed under vacuum to obtain ethyl (2-acetyl-phenyl)carbamate as white solid in 82% yield.

A mixture of 103 mg of ethyl (2-acetylphenyl)carbamate (0.5 mmol), an amine (0.6 mmol), such as benzylamine (64 mg) or (4-(piperidin-1-ylsulfonyl)phenyl)-methanamine (152.6 mg), and acetic acid (0.5 mmol) in toluene was heated at 120° C. for 4 h. After it had cooled, the residue was dried under vacuum. The crude material was diluted with ethyl acetate (10 mL) and washed with satd. NaHCO₃ solution (2×10 mL), DI water (2×10 mL), and satd. NaCl (15 mL) and dried over Na₂SO₄. It was then filtered and the solvent removed under vacuum. The mixture was purified by flash column chromatography on silica gel (50% EtOAc in hexane) to obtain the desired product as a white solid.

The following compounds were synthesized using procedure A: MXC001, MXC002, MXC003, MXC004, MXC005, MXC006, MXC009, MXC011, MXC012, MXC015, MXC016, MXC018, MXC022.

### General Procedure B

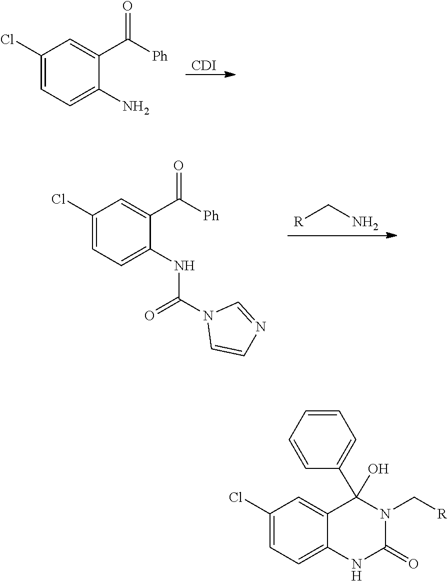

To a solution of 2-amino-5-chlorobenzophenone (1.15 g, 5 mmol) in dichloromethane (10 mL) was added 1,1′-carbonyldiimidizole (CDI, 891 mg, 5.5 mmol). The reaction mixture was heated to reflux for 6 h and then allowed to stir at 21° C. overnight. The heterogeneous mixture was recrystallized from 50% dichloromethane in hexane to give N-(2-benzoyl-4-chlorophenyl)-1H-imidazole-1-carboxamide in 68% yield as a white solid.

To a suspension of N-(2-benzoyl-4-chlorophenyl)-1H-imidazole-1-carboxamide (162 mg, 0.5 mmol) in THF (5.0 mL) was added an amine, such as benzylamine (64 mg, 0.6 mmol) or (4-(piperidin-1-ylsulfonyl)phenyl)methanamine (152.6 mg, 0.6 mmol), and the suspension became homogeneous as it was stirred at 50° C. for 24 hours. The reaction mixture was cooled, concentrated and the residue purified by flash column chromatography on silica gel (25% EtOAc in hexane) to afford the desired product as a white solid.

The following compounds were synthesized using procedure B: MXC007, MXC008, MXC010, MXC013, MXC036.

### General Procedure C

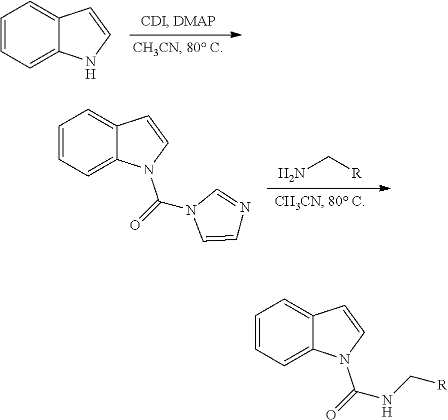

To a stirred solution of the indole (351.5 mg, 3 mmol) in anhydrous acetonitrile (5 mL) was added 1,1′-carbonyldiimidazole (CDI, 515.6 mg, 3.3 mmol, 1.06 equiv.) followed by 4-dimethylaminopyridine (DMAP, 10 mg). The resulting solution was stirred at reflux under argon until all the indole was consumed by TLC (or for 8 h). The resulting reaction mixture was cooled, the amine or alcohol (1.3 equiv.) was added and the mixture was again heated at reflux under argon overnight (16 h). The resulting reaction mixture was cooled and the solvent evaporated under vacuum>The residue was purified by flash column chromatography on silica gel using 4% ethyl acetate in hexane as eluent to afford the desired product.

The following compounds were synthesized using procedure C: MXC017, MXC027, MXC028, MXC029, MXC030, MXC031, MXC041, MXC042, MXC043, MXC044, MXC045, MXC046, MXC047, MXC057, MXC058, MXC071, MXC072, MXC073, MXC074, MXC075, MXC076, MXC077, MXC078, MXC079, MXC080, MXC081, MXC082, MXC083, MXC084, MXC085, MXC105.

### General Procedure D

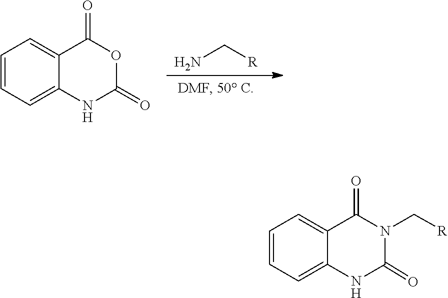

A solution of the amine (1 mmol) in dimethylformamide (DMF, 30 mL) was stirred while isatoic anhydride (1.3 mmol) was added portionwise over a period of 30 min. The temperature was maintained at 50° C. for an additional 30 min as the CO₂ was evaporated. The mixture was stirred at 21° C. for 2 h, then hot water (100 mL) was added and the mixture was kept at 21° C. without stirring for 6 h. The solid was collected by filtration, dried under high vacuum and purified by flash chromatography on silica gel using dichloromethane as eluent. If no solid precipitated during the reaction, then the mixture was exacted with ethyl acetate (200 mL). The organic layer was washed with water (300 mL×3) and brine and dried over Na₂SO₄. The solvent was removed under vacuum and the residue was purified by flash chromatography on silica gel using dichloromethane as eluent.

The following compounds were synthesized using procedure D: MXC019, MXC020, MXC021, MXC023, MXC024, MXC025, MXC026.

### General Procedure E

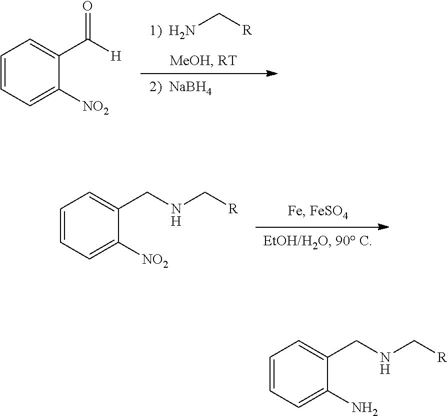

To a cooled solution of 2-nitrobenzaldehyde (302.2 mg, 2 mmol) in dry methanol (2 mL) was added Na₂SO₄ (340.9 mg, 2.4 mmol) under argon and the corresponding primary amine (2.2 mmol, 1.1 equiv.). After the mixture had stirred at 21° C. for 15 h, NaBH₄ (37.8 mg, 1.5 mmol) was added and the reaction was stirred for 3 h at 21° C. The mixture was concentrated under vacuum and the residue was dissolved in dichloromethane (15 mL), washed with water, dried and concentrated to afford the crude mixture that was purified by flash column chromatography on silica gel eluting with 50% ethyl acetate in hexane.

To a suspension of the nitroarene (1 mmol) from the previous step in aqueous ethanol (water/ethanol, 4/1) was added 3.0 mL of a mixture of iron (10 mmol) and iron (II) sulfate (1 mmol). The reaction was heated under reflux for 3 h, filtered through Celite and then extracted with dichloromethane (15 mL×3). The combined organic layers were washed with brine, dried over Na₂SO₄ and evaporated under vacuum. The residue was purified by flash column chromatography on silica gel eluting with 25% ethyl acetate in dichloromethane.

The following compounds were synthesized using procedure E: MXC014, MXC021, MXC032, MXC033, MXC034, MXC035, MXC039, MXC040, MXC048, MXC049, MXC050, MXC051, MXC052, MXC053, MXC054, MXC055, MXC056, MXC059, MXC060, MXC061, MXC062, MXC063, MXC064, MXC065, MXC066, MXC067, MXC068, MXC069, MXC070, MXC086, MXC087, MXC088, MXC089, MXC090, MXC091, MXC092, MXC093, MXC094, MXC095, MXC096, MXC097, MXC098, MXC099, MXC101, MXC102, MXC103, MXC104.

### General Procedure F

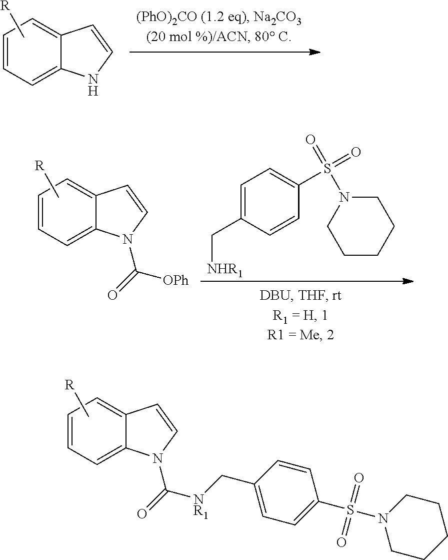

A mixture of the indole (0.50 mmol), diphenyl carbonate [(PhO)₂CO (0.51 mmol)] and sodium carbonate (Na₂CO₃, 20 mol %) in anhydrous acetonitrile (0.7 mL) in a sealed vial was stirred at reflux under argon for 1 d. The reaction mixture was cooled, concentrated and purified by flash column chromatography on silica gel using 5% diethyl ether in hexane as eluent to afford the desired carbamate product. This was then mixed with the corresponding amine 1 or 2 (1 eq) and DBU (1.1 eq) in anhydrous THF (1.0 M) in a sealed vial. After stirring at 21° C. for 22 h, the mixture was purified by flash column chromatography on silica gel using 33% ethyl acetate in hexane as eluent to afford the desired urea product.

The following compounds were synthesized using procedure F: MHD001, MHD002, MHD003, MHD004, MHD005, MHD006, MHD007, MHD008, MHD009, MHD0010, MHD011, MHD012, MHD013, MHD014, MHD015, MHD016, MHD017, MHD018, MHD019, MHD020, MHD021, MHD022, MHD023. Note: MHD001 and MHD008 were made with the secondary amine 2. The rest of the analogues were made with the primary amine 1.

### NMR Data for MXC001-MXC105:

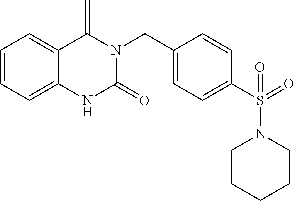 [SMILES: C=C1c2ccccc2NC(=O)N1Cc1ccc(S(=O)(=O)N2CCCCC2)cc1]

### 4-Methylene-3-(4-(piperidin-1-ylsulfonyl)benzyl)-3,4-dihydroquinazolin-2(1H)-one (Compound 2, also referred to as MXC001)

¹H NMR (400 MHz, CD₃OD) δ 8.27 (s, 1H), 7.71 (d, J=8.4 Hz, 2H), 7.54 (d, J=7.9 Hz, 1H), 7.44 (d, J=8.5 Hz, 2H), 7.28 (m, 1H), 7.03 (m, 1H), 6.79 (d, J=8.0 Hz, 1H), 5.17 (s, 2H), 4.81 (d, J=2.9 Hz, 1H), 4.12 (d, J=3.0 Hz, 1H), 2.97 (m, 4H), 1.63 (m, 4H), 1.42 (m, 2H).

¹³C NMR (101 MHz, CD₃OD) δ 151.2, 141.7, 140.0, 138.6, 135.2, 134.6, 130.4, 128.1, 126.9, 124.1, 123.2, 116.7, 114.7, 47.0, 46.9, 25.2, 23.5.

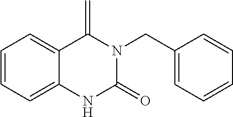 [SMILES: C=C1c2ccccc2NC(=O)N1Cc1ccccc1]

### 3-Benzyl-4-methylene-3,4-dihydroquinazolin-2(1H)-one (MXC002)

¹H NMR (400 MHz, CD₃OD) δ 8.51 (s, 1H), 7.52 (d, J=7.9 Hz, 1H), 7.32 (m, 3H), 7.24 (m, 3H), 6.98 (m, 1H), 6.79 (d, J=8.0 Hz, 1H), 5.12 (s, 2H), 4.78 (s, 1H), 4.22 (s, 1H).

¹³C NMR (126 MHz, Acetone-d₆) δ 150.2, 140.4, 137.4, 130.1, 128.4, 126.7, 126.5, 123.9, 122.2, 116.6, 114.6, 114.5, 84.8, 46.4.

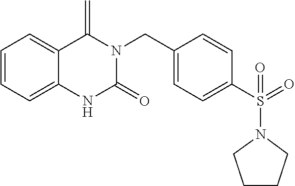 [SMILES: C=C1c2ccccc2NC(=O)N1Cc1ccc(S(=O)(=O)N2CCCC2)cc1]

### 4-Methylene-3-(4-(pyrrolidin-1-ylsulfonyl)benzyl)-3,4-dihydroquinazolin-2(1H)-one (MXC003)

¹H NMR (400 MHz, CD₃OD) δ 8.32 (s, 1H), 7.79 (d, J=8.4 Hz, 2H), 7.54 (d, J=7.6 Hz, 1H), 7.44 (d, J=8.5 Hz, 2H), 7.29 (m, 1H), 7.03 (m, 1H), 6.80 (d, J=7.8 Hz, 1H), 5.17 (s, 2H), 4.81 (d, J=2.9 Hz, 1H), 4.12 (d, J=3.0 Hz, 1H), 3.23 (m, 4H), 1.75 (m, 4H). ¹³C NMR (126 MHz, CDCl₃) δ 151.7, 141.7, 140.0, 138.6, 136.2, 135.4, 129.3, 128.5, 127.7, 124.4, 123.6, 115.2, 114.4, 47.9, 43.6, 25.2, 22.6.

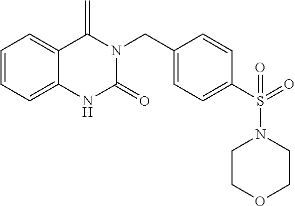 [SMILES: C=C1c2ccccc2NC(=O)N1Cc1ccc(S(=O)(=O)N2CCOCC2)cc1]

### 4-Methylene-3-(4-(morpholinosulfonyl)benzyl)-3,4-dihydroquinazolin-2(1H)-one (MXC004)

¹H NMR (400 MHz, CD₃OD) δ 8.33 (s, 1H), 7.71 (d, J=8.4 Hz, 2H), 7.54 (d, J=7.6 Hz, 1H), 7.47 (d, J=8.4 Hz, 2H), 7.29 (m, 1H), 7.04 (m, 1H), 6.80 (d, J=8.0 Hz, 1H), 5.18 (s, 2H), 4.82 (d, J=2.9 Hz, 1H), 4.11 (d, J=3.0 Hz, 1H), 3.74 (m, 4H), 2.98 (m, 4H).

¹³C NMR (126 MHz, CDCl₃) δ 151.7, 142.3, 140.5, 138.7, 135.4, 134.3, 129.6, 128.4, 127.7, 124.4, 123.8, 115.2, 114.3, 66.1, 45.9, 43.6.

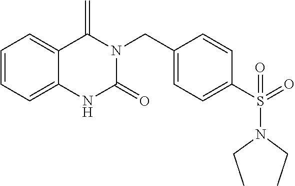 [SMILES: C=C1c2ccccc2NC(=O)N1Cc1ccc(S(=O)(=O)N(CC)CC)cc1]

### N,N-Diethyl-4-((4-methylene-2-oxo-1,4-dihydroquinazolin-3(2H)-yl)methyl)benzenesulfonamide (MXC005)

¹H NMR (400 MHz, CD₃OD) δ 8.77 (s, 1H), 7.76 (d, J=8.4 Hz, 2H), 7.52 (d, J=7.7 Hz, 1H), 7.41 (d, J=8.3 Hz, 2H), 7.18 (m, 1H), 7.02 (m, 1H), 6.80 (d, J=8.0 Hz, 1H), 5.16 (s, 2H), 4.79 (d, J=2.9 Hz, 1H), 4.10 (d, J=2.9 Hz, 1H), 3.22 (q, J=7.2 Hz, 4H), 1.12 (t, J=7.2 Hz, 6H).

¹³C NMR (126 MHz, CDCl₃) δ 151.4, 141.4, 140.0, 139.2, 136.9, 134.7, 130.4, 127.5, 126.9, 124.0, 123.1, 116.7, 114.8, 47.0, 42.1, 14.3.

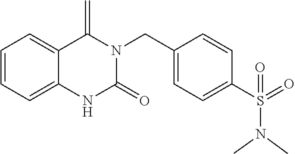 [SMILES: C=C1c2ccccc2NC(=O)N1Cc1ccc(S(=O)(=O)N(C)C)cc1]

### N,N-Dimethyl-4-((4-methylene-2-oxo-1,4-dihydroquinazolin-3(2H)-yl)methyl)benzenesulfonamide (MXC006)

¹H NMR (400 MHz, CD₃OD) δ 8.74 (s, 1H), 7.74 (d, J=8.4 Hz, 2H), 7.55 (d, J=8.0 Hz, 1H), 7.45 (d, J=8.5 Hz, 2H), 7.30 (m, 1H), 7.04 (m, 1H), 6.77 (d, J=8.0 Hz, 1H), 5.16 (s, 2H), 4.82 (d, J=3.0 Hz, 1H), 4.12 (d, J=3.0 Hz, 1H), 2.70 (s, 6H).

¹³C NMR (126 MHz, CDCl₃) δ 151.6, 141.8, 140.0, 138.6, 135.4, 134.7, 130.7, 128.7, 127.0, 124.1, 123.6, 115.2, 114.4, 47.2, 37.9.

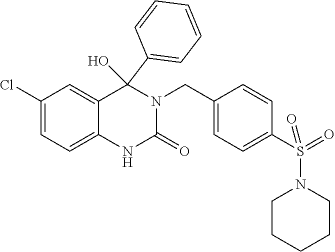 [SMILES: O=C1Nc2ccc(Cl)cc2C(O)(c2ccccc2)N1Cc1ccc(S(=O)(=O)N2CCCCC2)cc1]

### 6-Chloro-4-hydroxy-4-phenyl-3-(4-(piperidin-1-ylsulfonyl)benzyl)-3,4-dihydroquinazolin-2(1H)-one (MXC007)

¹H NMR (400 MHz, DMSO-d₆) δ 10.08 (s, 1H), 7.44 (m, 3H), 7.32 (m, 4H), 7.22 (m, 4H), 6.89 (d, J=8.6 Hz, 1H), 6.84 (d, J=2.3 Hz, 1H), 4.41 (d, J=16.2 Hz, 1H), 4.31 (d, J=16.1 Hz, 1H), 2.79 (m, 4H), 1.49 (m, 4H), 1.34 (m, 2H).

¹³C NMR (101 MHz, DMSO-d₆) δ 171.7, 152.1, 145.7, 144.9, 134.3, 133.4, 129.4, 128.7, 128.4, 127.5, 127.3, 126.9, 126.4, 125.0, 120.2, 116.0, 87.6, 47.0, 43.2, 25.1, 23.4. (one extra low-field peak due to impurity).

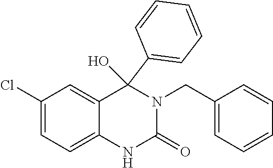 [SMILES: O=C1Nc2ccc(Cl)cc2C(O)(c2ccccc2)N1Cc1ccccc1]

### 3-Benzyl-6-chloro-4-hydroxy-4-phenyl-3,4-dihydroquinazolin-2(1H)-one (MXC008)

¹H NMR (400 MHz, DMSO-d₆) δ 9.97 (s, 1H), 7.39 (s, 1H), 7.36 (m, 2H), 7.27 (m, 2H), 7.19 (m, 2H), 7.08 (m, 5H), 6.87 (d, J=2.4 Hz, 1H), 6.85 (d, J=8.6 Hz, 1H), 4.32 (d, J=15.5 Hz, 1H), 4.15 (d, J=15.5 Hz, 1H).

¹³C NMR (101 MHz, DMSO-d₆) δ 152.2, 145.3, 140.2, 134.3, 129.2, 128.8, 128.3, 127.9, 127.7, 127.4, 127.0, 126.4, 126.2, 124.9, 115.9, 87.7, 46.3

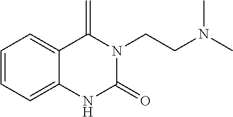 [SMILES: C=C1c2ccccc2NC(=O)N1CCN(C)C]

### 3-(2-(Dimethylamino)ethyl)-4-methylene-3,4-dihydroquinazolin-2(1H)-one (MXC009)

¹H NMR (500 MHz, CDCl₃) δ 9.16 (s, 1H), 7.53 (d, J=8.1 Hz, 1H), 7.22 (app. t, J=8.3 Hz, 1H,), 6.97 (m, 1H), 6.72 (d, J=8.0 Hz, 1H), 4.82 (d, J=2.6 Hz, 1H), 4.35 (d, J=2.7 Hz, 1H), 3.86 (t, J=7.5 Hz, 2H), 2.61 (t, J=7.6 Hz, 2H), 2.34 (s, 6H).

¹³C NMR (126 MHz, CDCl₃) δ 150.6, 139.5, 134.8, 130.3, 124.1, 123.1, 122.3, 116.7, 114.6, 58.4, 53.4, 44.3.

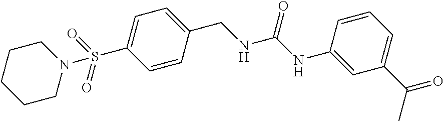 [SMILES: CC(=O)c1cccc(NC(=O)NCc2ccc(S(=O)(=O)N3CCCCC3)cc2)c1]

### 1-(3-Acetylphenyl)-3-(4-(piperidin-1-ylsulfonyl)benzyl)urea (MXC010)

¹H NMR (500 MHz, CDCl₃) δ 7.90 (s, 1H), 7.74 (d, J=8.1 Hz, 1H), 7.65 (s, 1H), 7.55 (m, 3H), 7.35 (m, 3H), 6.09 (t, J=5.9 Hz, 1H). 4.47 (d, J=5.9 Hz, 2H), 2.95 (m, 4H), 2.54 (s, 3H), 1.59 (m, 4H), 1.41 (m, 2H).

¹³C NMR (126 MHz, CDCl₃) δ 198.7, 155.8, 144.8, 139.6, 137.6, 134.4, 129.3, 127.8, 127.6, 124.0, 122.8, 118.5, 46.9, 43.2, 26.8, 25.1, 23.3.

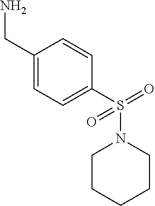 [SMILES: NCc1ccc(S(=O)(=O)N2CCCCC2)cc1]

### (4-(Piperidin-1-ylsulfonyl)phenyl)methanamine (MXC011)

¹H NMR (500 MHz, CDCl₃) δ 7.69 (d, J=8.1 Hz, 2H), 7.47 (d, J=8.1 Hz, 2H), 3.96 (s, 2H), 2.97 (m, 4H), 1.62 (m, 4H), 1.40 (m, 2H).

¹³C NMR (126 MHz, CDCl₃) δ 148.1, 145.7, 127.9, 127.5, 46.9, 45.8, 25.1, 23.5.

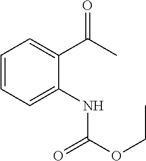

### Ethyl (2-acetylphenyl)carbamate (MXC012)

¹H NMR (500 MHz, CDCl₃) δ 11.13 (s, 1H), 8.47 (dd, J=8.5, 1.0 Hz), 7.86 (dd, J=8.0, 1.5 Hz, 1H), 7.52 (ddd, J=8.5, 7.3, 1.3 Hz, 1H), 7.04 (m, 1H), 4.21 (q, J=7.1 Hz, 2H), 2.64 (s, 3H), 1.31 (t, J=7.1 Hz, 3H).

¹³C NMR (126 MHz, CDCl₃) δ 202.3, 153.9, 141.5, 135.0, 131.7, 121.3, 119.2, 61.2, 28.6, 14.5. (One low-field carbon not observed.)

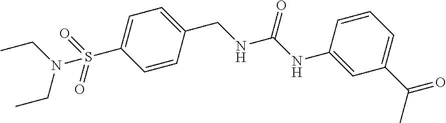 [SMILES: CCN(CC)S(=O)(=O)c1ccc(CNC(=O)Nc2cccc(C(C)=O)c2)cc1]

### 4-((3-(3-Acetylphenyl)ureido)methyl)-N,N-diethylbenzenesulfonamide (MXC013)

¹H NMR (500 MHz, CDCl₃) δ 7.89 (s, 1H), 7.72 (d, J=8.0 Hz, 1H), 7.66 (s, 1H), 7.55 (m, 3H), 7.35 (m, 3H), 6.09 (t, J=5.8 Hz, 1H). 4.47 (d, J=5.8 Hz, 2H), 3.16 (q, J=7.1 Hz, 4H), 1.08 (t, J=7.1 Hz, 6H).

¹³C NMR (126 MHz, CDCl₃) δ 198.7, 155.9, 144.4, 139.7, 138.4, 137.5, 129.2, 127.7, 127.1, 123.9, 122.7, 118.5, 42.2, 31.7, 26.7, 14.2.

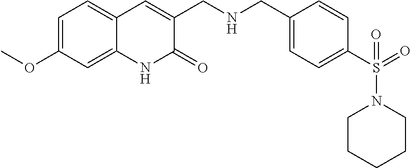 [SMILES: COc1ccc2cc(CNCc3ccc(S(=O)(=O)N4CCCCC4)cc3)c(=O)[nH]c2c1]

### 7-Methoxy-3-(((4-(piperidin-1-ylsulfonyl)benzyl)amino)methyl)quinolin-2(1H)-one (MXC014)

¹H NMR (500 MHz, CDCl₃) δ 10.59 (s, 1H), 7.71 (d, J=8.5 Hz, 2H), 7.69 (s, 1H), 7.54 (d, J=8.2 Hz, 2H), 7.46 (d, J=8.7 Hz, 1H), 7.24 (s, 1H), 6.83 (d, J=8.7 Hz, 1H), 6.69 (br s, 1H), 3.92 (s, 2H), 3.87 (s, 3H), 3.82 (s, 2H), 2.96 (m, 4H), 1.67 (m, 4H), 1.41 (m, 2H).

¹³C NMR (126 MHz, CDCl₃) δ 164.2, 161.5, 144.2, 139.6, 138.9, 135.0, 129.8, 128.9, 128.7, 127.8, 114.1, 112.2, 98.3, 55.5, 52.2, 48.8, 46.9, 25.1, 23.4.

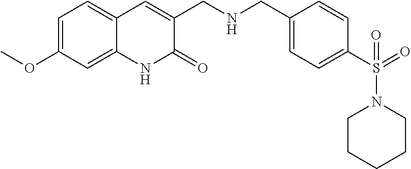 [SMILES: COc1ccc2cc(CNCc3ccc(S(=O)(=O)N4CCCCC4)cc3)c(=O)[nH]c2c1]

### 4-Methylene-3-(4-(piperidine-1-carbonyl)benzyl)-3,4-dihydroquinazolin-2(1H)-one (MXC015)

¹H NMR (500 MHz, CDCl₃) δ 8.24 (s, 1H), 7.52 (d, J=7.9 Hz, 1H), 7.34 (m, 3H), 7.25 (m, 2H), 7.00 (m, 1H), 6.78 (d, J=8.0 Hz, 1H), 5.11 (s, 2H), 4.78 (d, J=2.8 Hz, 1H), 4.17 (d, J=2.9 Hz, 1H), 3.69 (m, 2H), 3.33 (m, 2H), 1.66 (m, 4H), 1.48 (m, 2H).

¹³C NMR (126 MHz, CDCl₃) δ 170.2, 151.4, 140.2, 138.8, 138.3, 135.5, 128.7, 128.3, 127.4, 126.9, 123.2, 120.0, 115.3, 114.4, 48.8, 43.7, 25.6, 24.3.

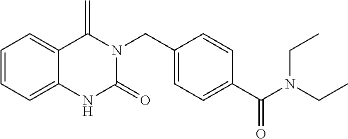 [SMILES: C=C1c2ccccc2NC(=O)N1Cc1ccc(C(=O)N(CC)CC)cc1]

### N,N-Diethyl-4-((4-methylene-2-oxo-1,4-dihydroquinazolin-3(2H)-yl)methyl)benzamide (MXC016)

¹H NMR (500 MHz, CDCl₃) δ 9.11 (s, 1H), 7.50 (d, J=7.6 Hz, 1H), 7.33 (m, 4H), 7.23 (m, 1H), 6.98 (m, 1H), 6.81 (dd, J=8.0, 0.9 Hz, 1H), 5.12 (s, 2H), 4.77 (d, J=2.7 Hz, 1H), 4.15 (d, J=2.9 Hz, 1H), 3.52 (m, 2H), 3.25 (m, 2H), 1.22 (m, 3H), 1.09 (m, 3H).

¹³C NMR (126 MHz, CDCl₃) δ 171.3, 151.7, 140.0, 138.9, 1381, 136.2, 135.1, 128.9, 128.2, 126.9, 126.4, 123.2, 115.4, 114.4, 47.1, 43.3, 14.2.

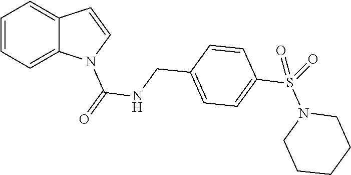 [SMILES: O=C(NCc1ccc(S(=O)(=O)N2CCCCC2)cc1)n1ccc2ccccc21]

### N-(4-(Piperidin-1-ylsulfonyl)benzyl)-1H-indole-1-carboxamide (MXC017)

¹H NMR (500 MHz, CDCl₃) δ 8.20 (d, J=8.3 Hz, 1H), 7.59 (d, J=7.8 Hz, 1H), 7.56 (d, J=3.7 Hz, 1H), 7.52 (d, J=8.2 Hz, 2H), 7.40 (d, J=8.2 Hz, 2H), 7.31 (t, J=7.7 Hz, 1H), 7.23 (t, J=7.5 Hz, 1H), 6.66 (t, J=6.0 Hz, 1H), 6.64 (d, J=3.6 Hz, 1H), 4.69 (d, J=5.9 Hz, 2H), 2.91 (m, 4H), 1.62 (m, 4H), 1.36 (m, 2H).

¹³C NMR (126 MHz, CDCl₃) δ 152.3, 143.3, 135.3, 135.2, 130.2, 127.9, 127.8, 124.4, 123.6, 122.5, 121.2, 114.5, 107.6, 46.9, 44.0, 25.1, 23.4.

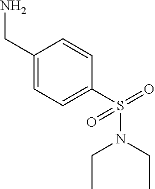 [SMILES: CCN(CC)S(=O)(=O)c1ccc(CN)cc1]

### 4-(Aminomethyl)-N,N-diethylbenzenesulfonamide (MXC018)

¹H NMR (500 MHz, CDCl₃) δ 7.74 (d, J=8.3 Hz, 2H), 7.43 (d, J=8.3 Hz, 2H), 3.93 (s, 2H), 3.21 (q, J=7.1 Hz, 4H), 1.89 (br s, 2H), 1.10 (t, J=7.2 Hz, 6H).

¹³C NMR (126 MHz, CDCl₃) δ 147.6, 138.7, 127.6, 127.3, 45.8, 42.1, 14.2.

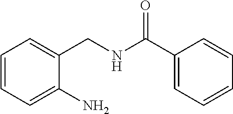 [SMILES: Nc1ccccc1CNC(=O)c1ccccc1]

### 2-Amino-N-benzylbenzamide (MXC019)

¹H NMR (500 MHz, CDCl₃) δ 7.36 (m, 4H), 7.31 (m, 2H), 7.21 (t, J=7.6 Hz, 1H), 6.69 (d, J=8.2 Hz, 1H), 6.63 (t, J=7.4 Hz, 1H), 6.32 (s, 1H), 5.55 (br s, 2H), 4.61 (d, J=5.6 Hz, 2H).

¹³C NMR (126 MHz, CDCl₃) δ 168.9, 146.8, 138.1, 132.5, 128.8, 127.8, 127.6, 127.1, 118.4, 118.1, 116.9, 43.8.

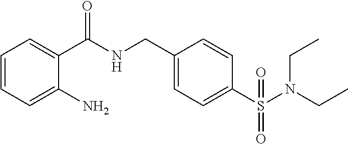 [SMILES: CCN(CC)S(=O)(=O)c1ccc(CNC(=O)c2ccccc2N)cc1]

### 2-Amino-N-(4-(N,N-diethylsulfamoyl)benzyl)benzamide (MXC020)

¹H NMR (500 MHz, CDCl₃) δ 7.70 (d, J=7.5 Hz, 2H), 7.40 (m, 3H), 7.22 (t, J=7.6 Hz, 1H), 6.72 (s, 1H), 6.69 (d, J=8.2 Hz, 1H), 6.64 (t, J=7.5 Hz, 1H), 5.57 (br s, 2H), 4.64 (d, J=6.0 Hz, 2H), 3.20 (q, J=7.1 Hz, 4H), 1.12 (t, J=7.1 Hz, 6H).

¹³C NMR (126 MHz, CDCl₃) δ 169.5, 148.9, 143.6, 138.8, 132.6, 127.8, 127.4, 127.2, 117.4, 116.6, 115.3, 42.8, 42.2, 14.3.

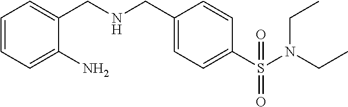 [SMILES: CCN(CC)S(=O)(=O)c1ccc(CNCc2ccccc2N)cc1]

### 4-(((2-Aminobenzyl)amino)methyl)-N,N-diethylbenzenesulfonamide (MXC021)

¹H NMR (500 MHz, CDCl₃) δ 7.76 (d, J=8.2 Hz, 2H), 7.43 (d, J=8.1 Hz, 2H), 7.10 (t, J=7.1 Hz, 1H), 7.01 (d, J=7.2 Hz, 1H), 6.69 (m, 2H), 3.85 (s, 2H), 3.83 (s, 2H), 3.23 (q, J=7.1 Hz, 4H), 1.13 (t, J=7.1 Hz, 6H).

¹³C NMR (126 MHz, CDCl₃) δ 146.6, 143.4, 139.4, 130.5, 129.0, 128.9, 127.2, 122.1, 118.1, 116.2, 52.0, 51.7, 42.1, 14.2.

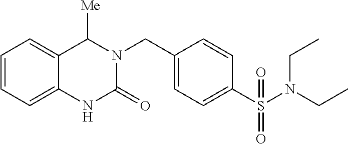 [SMILES: CCN(CC)S(=O)(=O)c1ccc(CN2C(=O)Nc3ccccc3C2C)cc1]

### N,N-Diethyl-4-((4-methyl-2-oxo-1,4-dihydroquinazolin-3(2H)-yl)methyl)benzenesulfonamide (MXC022)

¹H NMR (500 MHz, CDCl₃) δ 8.04 (s, 1H), 7.75 (d, J=8.3 Hz, 2H), 7.43 (d, J=8.2 Hz, 2H), 7.17 (m, 1H), 6.94 (d, J=4.3 Hz, 2H), 6.77 (d, J=7.9 Hz, 1H), 5.29 (d, J=16.0 Hz, 1H), 4.39 (q, J=6.5 Hz, 1H), 4.28 (d, J=16.0 Hz, 1H), 3.22 (q, J=7.1 Hz, 4H), 1.36 (d, J=6.6 Hz, 3H), 1.12 (t, J=7.1 Hz, 6H).

¹³C NMR (126 MHz, CDCl₃) δ 154.7, 142.5, 139.5, 135.8, 128.3, 128.0, 127.4, 125.3, 123.1, 122.4, 114.1, 54.7, 47.8, 42.1, 21.3, 14.2.

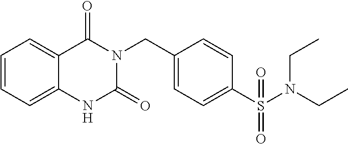 [SMILES: CCN(CC)S(=O)(=O)c1ccc(Cn2c(=O)[nH]c3ccccc3c2=O)cc1]

### 4-((2,4-Dioxo-1,4-dihydroquinazolin-3(2H1)-yl)methyl)-N,N-diethylbenzenesulfonamide (MXC023)

¹H NMR (500 MHz, CDCl₃) δ 10.34 (s, 1H), 8.13 (d, J=7.9 Hz, 1H), 7.75 (d, J=8.3 Hz, 2H), 7.63 (m, 3H), 7.25 (m, 1H), 7.08 (d, J=8.1 Hz, 1H), 5.31 (s, 2H), 3.19 (q, J=7.1 Hz, 4H), 1.11 (t, J=7.1 Hz, 6H).

¹³C NMR (126 MHz, CDCl₃) δ 162.3, 152.1, 141.3, 139.6, 138.5, 135.5, 129.4, 128.5, 127.3, 123.8, 115.2, 114.4, 43.7, 42.2, 14.3.

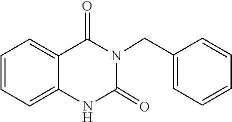 [SMILES: O=c1[nH]c2ccccc2c(=O)n1Cc1ccccc1]

### 3-Benzylquinazoline-2,4(1H,3H)-dione (MXC024)

¹H NMR (500 MHz, CDCl₃) δ 10.09 (s, 1H), 8.14 (d, J=7.9 Hz 1H), 7.60 (m, 1H), 7.53 (d, J=7.3 Hz, 2H), 7.31 (app. t, J=7.4 Hz, 2H), 7.25 (m, 2H), 7.06 (d, J=8.1 Hz, 1H), 5.28 (s, 2H).

¹³C NMR (126 MHz, CDCl₃) δ 162.3, 152.1, 138.5, 136.9, 135.1, 128.9, 128.6, 128.5, 127.7, 123.5, 115.0, 114.7, 44.2.

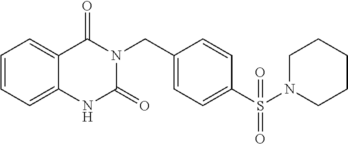 [SMILES: O=c1[nH]c2ccccc2c(=O)n1Cc1ccc(S(=O)(=O)N2CCCCC2)cc1]

### 3-(4-(Piperidin-1-ylsulfonyl)benzyl)quinazoline-2,4(1H,3H)-dione (MXC025)

¹H NMR (500 MHz, CDCl₃) δ 9.72 (s, 1H), 8.15 (dd, J=8.0, 1.4 Hz, 1H), 7.69 (m, 5H), 7.26 (m, 1H), 7.06 (d, J=8.0 Hz, 1H), 5.32 (s, 2H), 2.95 (m, 4H), 1.61 (m, 4H), 1.39 (m, 2H).

¹³C NMR (126 MHz, CDCl₃) δ 162.2, 151.5, 141.6, 138.3, 135.6, 135.5, 129.4, 128.7, 127.9, 123.8, 114.9, 114.4, 46.9, 43.7, 25.2, 23.5.

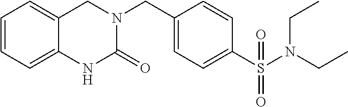 [SMILES: CCN(CC)S(=O)(=O)c1ccc(CN2Cc3ccccc3NC2=O)cc1]

### N,N-Diethyl-4-((2-oxo-1,4-dihydroquinazolin-3(2H)-yl)methyl)benzenesulfonamide (MXC026)

¹H NMR (500 MHz, CDCl₃) δ 8.63 (s, 1H), 7.78 (d, J=8.3 Hz, 2H), 7.48 (d, J=8.3 Hz, 2H), 7.15 (app. t, J=7.6 Hz, 1H), 6.95 (d, J=7.2 Hz, 1H), 6.91 (app. t, J=7.4 Hz, 1H), 6.78 (d, J=7.9 Hz, 1H), 4.74 (s, 2H), 4.37 (s, 2H), 3.22 (q, J=7.2 Hz, 4H), 1.12 (t, J=7.2 Hz, 6H).

¹³C NMR (126 MHz, CDCl₃) δ 155.0, 141.6, 139.7, 136.6, 128.4, 127.5, 125.5, 122.2, 117.0, 114.1, 50.1, 48.5, 42.2, 14.3 (one low-field carbon signal not observed).

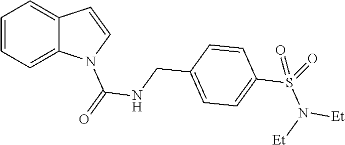 [SMILES: CCN(CC)S(=O)(=O)c1ccc(CNC(=O)n2ccc3ccccc32)cc1]

### N-(4-(N,N-Diethylsulfamoyl)benzyl)-1H-indole-1-carboxamide (MXC027)

¹H NMR (500 MHz, CDCl₃) δ 8.22 (d, J=8.2 Hz, 1H), 7.58 (m, 2H), 7.53 (d, J=8.2 Hz, 2H), 7.33 (d, J=8.2 Hz, 2H), 7.28 (m, 1H), 7.22 (app. t, J=7.3 Hz, 1H), 6.88 (t, J=5.7 Hz, 1H), 6.61 (d, J=3.6 Hz, 1H), 4.63 (d, J=5.7 Hz, 2H), 3.15 (q, J=7.0 Hz, 4H), 1.09 (t, J=7.0 Hz, 6H).

¹³C NMR (126 MHz, CDCl₃) δ 152.7, 143.5, 138.5, 135.6, 130.1, 127.8, 127.0, 124.3, 124.0, 122.4, 121.0, 114.9, 107.5, 43.8, 42.3, 14.3.

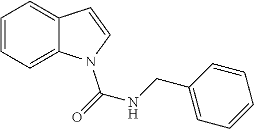 [SMILES: O=C(NCc1ccccc1)n1ccc2ccccc21]

### N-Benzyl-1H-indole-1-carboxamide (MXC028)

¹H NMR (500 MHz, CDCl₃) δ 8.13 (d, J=8.2 Hz, 1H), 7.61 (d, J=7.7 Hz, 1H), 7.46 (d, J=3.6 Hz, 1H), 7.33 (m, 6H), 7.24 (t, J=7.4 Hz, 1H), 6.60 (d, J=3.6 Hz, 1H), 6.17 (br. s, 1H), 4.60 (d, J=5.5 Hz, 2H).

¹³C NMR (126 MHz, CDCl₃) δ 152.5, 138.0, 135.4, 130.1, 128.8, 127.8, 127.7, 124.2, 124.1, 122.4, 121.1, 114.5, 107.2, 44.8.

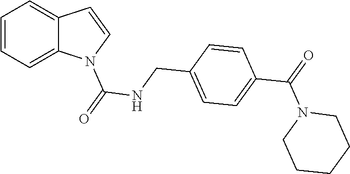 [SMILES: O=C(c1ccc(CNC(=O)n2ccc3ccccc32)cc1)N1CCCCC1]

### N-(4-(Piperidine-1-carbonyl)benzyl)-1H-indole-1-carboxamide (MXC029)

¹H NMR (500 MHz, CDCl₃) δ 8.25 (d, J=8.3 Hz, 1H), 7.72 (d, J=3.6 Hz, 1H), 7.62 (t, J=5.7 Hz, 1H), 7.57 (d, J=7.7 Hz, 1H), 7.27 (m, 1H), 7.17 (m, 5H), 6.58 (d, J=3.5 Hz, 1H), 4.47 (d, J=5.7 Hz, 2H), 3.69 (m, 2H), 3.29 (m, 2H), 1.66 (m, 4H), 1.46 (m, 2H).

¹³C NMR (126 MHz, CDCl₃) δ 170.5, 152.9, 140.4, 135.8, 134.5, 130.0, 126.9, 126.7, 124.4, 124.0, 122.2, 120.8, 115.2, 106.9, 48.9, 43.8, 25.7, 24.5.

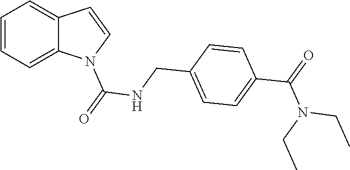 [SMILES: CCN(CC)C(=O)c1ccc(CNC(=O)n2ccc3ccccc32)cc1]

### N-(4-(Diethylcarbamoyl)benzyl)-1H-indole-1-carboxamide (MXC030)

¹H NMR (500 MHz, CDCl₃) δ 8.23 (d, J=8.3 Hz, 1H), 7.73 (d, J=3.6 Hz, 1H), 7.58 (d, J=7.8 Hz, 1H), 7.35 (s, 1H), 7.29 (app. t, J=7.7 Hz, 1H), 7.20 (m, 5H), 6.60 (d, J=3.5 Hz, 1H), 4.53 (d, J=5.7 Hz, 2H), 3.54 (m, 2H), 3.23 (m, 2H), 1.26 (t, J=7.1 Hz, 3H), 1.08 (m, 3H).

¹³C NMR (126 MHz, CDCl₃) δ 171.7, 152.9, 140.2, 135.8, 135.1, 130.0, 126.9, 126.1, 124.5, 124.0, 122.0, 120.8, 115.2, 106.9, 43.8, 39.7, 29.7, 14.2, 12.9.

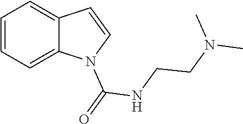 [SMILES: CN(C)CCNC(=O)n1ccc2ccccc21]

### N-(2-(Dimethylamino)ethyl)-1H-indole-1-carboxamide (MXC031)

¹H NMR (500 MHz, CDCl₃) δ 8.13 (d, J=8.4 Hz, 1H), 7.59 (d, J=7.8 Hz, 1H), 7.54 (d, J=3.6 Hz, 1H), 7.32 (app. t, J=7.7 Hz, 1H), 7.22 (app. t, J=7.5 Hz, 1H), 6.64 (br. s, 1H), 6.61 (d, J=3.6 Hz, 1H), 3.55 (t, J=5.8 Hz, 2H), 2.60 (t, J=5.8 Hz, 2H), 2.33 (s, 6H).

¹³C NMR (126 MHz, CDCl₃) δ 152.3, 135.2, 130.2, 124.4, 124.1, 122.2, 121.1, 114.3, 106.8, 57.7, 45.0, 37.9.

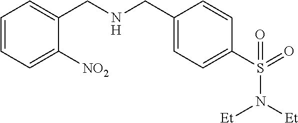 [SMILES: CCN(CC)S(=O)(=O)c1ccc(CNCc2ccccc2[N+](=O)[O-])cc1]

### N,N-Diethyl-4-(((2-nitrobenzyl)amino)methyl)benzenesulfonamide (MXC032)

¹H NMR (500 MHz, CDCl₃) δ 7.95 (d, J=8.0 Hz, 1H), 7.76 (d, J=8.3 Hz, 2H), 7.58 (m, 2H), 7.47 (d, J=8.3 Hz, 2H), 7.43 (m, 1H), 4.06 (s, 2H), 3.88 (s, 2H), 3.23 (q, J=7.1 Hz, 4H), 1.13 (t, J=7.2 Hz, 6H).

¹³C NMR (126 MHz, CDCl₃) δ 149.2, 144.6, 139.1, 134.9, 133.3, 131.4, 128.6, 128.3, 127.2, 124.9, 52.8, 50.3, 42.1, 14.2.

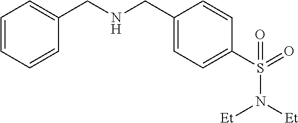 [SMILES: CCN(CC)S(=O)(=O)c1ccc(CNCc2ccccc2)cc1]

### 4-((Benzylamino)methyl)-N,N-diethylbenzenesulfonamide (MXC033)

¹H NMR (500 MHz, CDCl₃) δ 7.76 (d, J=8.4 Hz, 2H), 7.47 (d, J=8.4 Hz, 2H), 7.33 (m, 4H), 7.26 (m, 1H), 3.86 (s, 1H), 3.80 (s, 2H), 3.23 (q, J=7.2 Hz, 4H), 1.12 (t, J=7.2 Hz, 6H).

¹³C NMR (126 MHz, CDCl₃) δ 144.8, 139.5, 138.9, 128.7, 128.5, 128.3, 127.3, 127.1, 53.1, 52.3, 42.1, 14.2.

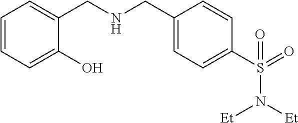 [SMILES: CCN(CC)S(=O)(=O)c1ccc(CNCc2ccccc2O)cc1]

### N,N-Diethyl-4-(((2-hydroxybenzyl)amino)methyl)benzenesulfonamide (MXC034)

¹H NMR (500 MHz, CDCl₃) δ 7.77 (d, J=8.4 Hz, 2H), 7.42 (d, J=8.4 Hz, 2H), 7.18 (app. td, J=8.0, 1.7 Hz, 1H), 6.98 (dd, J=7.4, 1.3 Hz, 1H), 6.85 (dd, J=8.1, 1.0 Hz, 1H), 6.79 (td, J=7.4, 1.1 Hz, 1H), 4.02 (s, 2H), 3.87 (s, 2H), 3.23 (q, J=7.2 Hz, 4H), 1.12 (t, J=7.2 Hz, 6H).

¹³C NMR (126 MHz, CDCl₃) δ 156.6, 141.4, 138.7, 128.2, 128.1, 128.0, 126.4, 120.3, 118.4, 115.4, 50.6, 50.4, 41.1, 13.2.

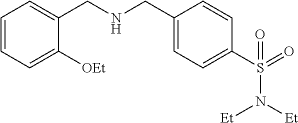 [SMILES: CCOc1ccccc1CNCc1ccc(S(=O)(=O)N(CC)CC)cc1]

### 4-(((2-Ethoxybenzyl)amino)methyl)-N,N-diethylbenzenesulfonamide (MXC035)

¹H NMR (500 MHz, CDCl₃) δ 7.75 (d, J=8.4 Hz, 2H), 7.47 (d, J=8.4 Hz, 2H), 7.22 (m, 2H), 6.90 (td, J=7.4, 1.0 Hz, 1H), 6.85 (d, J=8.1 Hz, 1H), 4.05 (q, J=7.0 Hz, 2H), 3.82 (s, 2H), 3.80 (s, 2H), 3.22 (q, J=7.2 Hz, 4H), 1.40 (t, J=7.0 Hz, 3H), 1.12 (t, J=7.2 Hz, 6H).

¹³C NMR (126 MHz, CDCl₃) δ 157.1, 145.0, 138.8, 130.1, 128.7, 128.6, 127.4, 127.1, 120.3, 111.2, 63.5, 52.1, 48.8, 42.1, 15.0, 14.2.

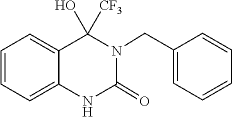 [SMILES: O=C1Nc2ccccc2C(O)(C(F)(F)F)N1Cc1ccccc1]

### 3-Benzyl-4-hydroxy-4-(trifluoromethyl)-3,4-dihydroquinazolin-2(1H)-one (MXC036)

¹H NMR (500 MHz, CDCl₃) δ 7.96 (m, 1H), 7.54 (m, 1H), 7.29 (m, 5H), 7.09 (m, 1H), 6.74 (m, 1H), 5.28 (d, J=15.3 Hz, 1H), 4.72 (d, J=15.3 Hz, 1H), 3.43 (s, 1H), 2.04 (s, 1H).

¹³C NMR (126 MHz, CDCl₃) δ 152.5, 139.5, 135.4, 131.8, 128.6, 127.3, 127.2, 123.6 (q, J=291.3 Hz), 122.6, 116.4, 114.3, 85.0 (q, J=32.8 Hz), 29.7.

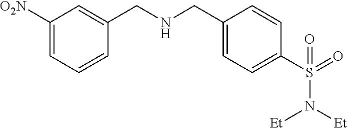 [SMILES: CCN(CC)S(=O)(=O)c1ccc(CNCc2cccc([N+](=O)[O-])c2)cc1]

### N,N-Diethyl-4-(((3-nitrobenzyl)amino)methyl)benzenesulfonamide (MXC039)

¹H NMR (500 MHz, CDCl₃) δ 8.23 (s, 1H), 8.09 (d, J=7.7 Hz, 1H), 7.75 (d, J=8.1 Hz, 2H), 7.68 (d, J=7.5 Hz, 1H), 7.49 (m, 3H), 3.90 (s, 2H), 3.87 (s, 2H), 3.22 (q, J=7.2 Hz, 4H), 1.11 (t, J=7.2 Hz, 6H).

¹³C NMR (126 MHz, CDCl₃) δ 148.4, 144.4, 142.0, 139.2, 134.3, 129.4, 128.6, 127.2, 122.9, 122.3, 52.5, 52.3, 42.1, 14.2.

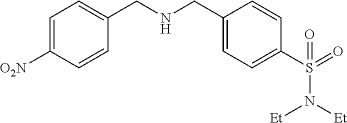 [SMILES: CCN(CC)S(=O)(=O)c1ccc(CNCc2ccc([N+](=O)[O-])cc2)cc1]

### N,N-Diethyl-4-(((4-nitrobenzyl)amino)methyl)benzenesulfonamide (MXC040)

¹H NMR (500 MHz, CDCl₃) δ 8.18 (d, J=8.4 Hz, 2H). 7.76 (d, J=8.0 Hz, 2H), 7.53 (d, J=8.4 Hz, 2H), 7.47 (d, J=8.0 Hz, 2H), 3.92 (s, 2H), 3.87 (s, 2H), 3.23 (q, J=7.1 Hz, 4H), 1.12 (t, J=7.1 Hz, 6H).

¹³C NMR (126 MHz, CDCl₃) δ 147.3, 146.4, 143.3, 139.5, 129.0, 128.8, 127.3, 123.7, 52.2, 52.0, 42.1, 14.2.

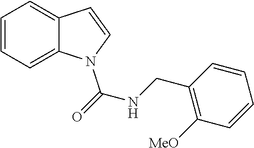 [SMILES: COc1ccccc1CNC(=O)n1ccc2ccccc21]

### N-(2-Methoxybenzyl)-1H-indole-1-carboxamide (MXC041)

¹H NMR (500 MHz, CDCl₃) δ 8.05 (dd, J=8.3, 0.8 Hz, 1H), 7.60 (d, J=7.7 Hz, 1H), 7.49 (d, J=3.7 Hz, 1H), 7.38 (dd, J=7.4, 1.6 Hz, 1H), 7.31 (m, 2H), 7.22 (m, 1H), 6.96 (app. td, J=7.5, 1.0 Hz, 1H), 6.92 (d, J=8.2 Hz, 1H), 6.59 (dd, J=3.6, 0.7 Hz, 1H), 6.37 (s, 1H), 4.65 (d, J=5.8 Hz, 2H), 3.90 (s, 3H).

¹³C NMR (126 MHz, CDCl₃) δ 157.6, 152.2, 135.1, 130.3, 129.8, 129.2, 125.8, 124.5, 124.0, 122.2, 121.2, 128.8, 114.1, 110.5, 106.8, 55.4, 41.0.

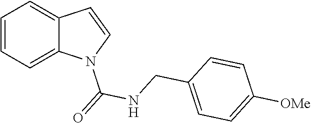 [SMILES: COc1ccc(CNC(=O)n2ccc3ccccc32)cc1]

### N-(4-Methoxybenzyl)-1H-indole-1-carboxamide (MXC042)

¹H NMR (500 MHz, CDCl₃) δ 8.09 (dd, J=8.3, 0.8 Hz, 1H), 7.59 (d, J=7.7 Hz, 1H), 7.44 (d, J=3.7 Hz, 1H), 7.30 (m, 3H), 7.22 (m, 1H), 6.88 (d, J=8.7 Hz, 2H), 6.59 (dd, J=3.7, 0.7 Hz, 1H), 5.92 (s, 1H), 4.56 (d, J=5.5 Hz, 2H), 3.80 (s, 3H).

¹³C NMR (126 MHz, CDCl₃) δ 159.2, 152.3, 135.3, 130.2, 129.9, 129.2, 124.2, 124.1, 122.4, 121.2, 114.4, 114.2, 107.2, 55.3, 44.4.

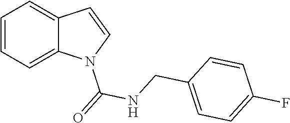 [SMILES: O=C(NCc1ccc(F)cc1)n1ccc2ccccc21]

### N-(4-Fluorobenzyl)-1H-indole-1-carboxamide (MXC043)

¹H NMR (500 MHz, CDCl₃) δ 8.08 (dd, J=8.3, 0.8 Hz, 1H), 7.59 (d, J=7.5 Hz, 1H), 7.44 (d, J=3.7 Hz, 1H), 7.32 (m, 3H), 7.23 (m, 1H), 7.02 (t, J=8.7 Hz, 2H), 6.60 (dd, J=3.7, 0.7 Hz, 1H), 6.03 (s, 1H), 4.57 (d, J=5.7 Hz, 2H).

¹³C NMR (126 MHz, CDCl₃) δ 162.3 (d, J=246.1 Hz), 152.2, 135.2, 133.6 (d, J=3.2 Hz), 130.2, 129.5, 129.4, 124.3, 124.0, 122.5, 121.3, 115.7 (d, J=21.5 Hz), 114.3, 44.1.

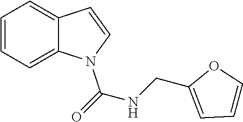 [SMILES: O=C(NCc1ccco1)n1ccc2ccccc21]

### N-(Furan-2-ylmethyl)-1H-indole-1-carboxamide (MXC044)

¹H NMR (500 MHz, CDCl₃) δ 8.10 (dd, J=8.4, 0.8 Hz, 1H), 7.59 (d, J=7.8 Hz, 1H), 7.45 (d, J=3.7 Hz, 1H), 7.39 (dd, J=1.8, 0.9 Hz, 1H), 7.31 (m, 1H), 7.23 (m, 1H), 6.61 (dd, J=3.7, 0.6 Hz, 1H), 6.35 (dd, J=3.2, 1.8 Hz, 1H), 6.34 (dd, J=3.2, 0.7 Hz, 1H), 5.96 (s, 1H), 4.64 (d, J=5.5 Hz, 2H).

¹³C NMR (126 MHz, CDCl₃) δ 152.2, 150.9, 142.4, 135.3, 130.2, 124.3, 124.1, 122.4, 121.2, 114.4, 110.6, 107.9, 107.3, 37.8.

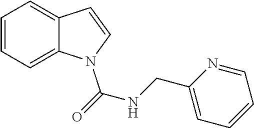 [SMILES: O=C(NCc1ccccn1)n1ccc2ccccc21]

### N-(Pyridin-2-ylmethyl)-1H-indole-1-carboxamide (MXC045)

¹H NMR (500 MHz, CDCl₃) δ 8.57 (d, J=4.9 Hz, 1H), 8.22 (dd, J=8.4, 0.8 Hz, 1H), 7.70 (app. td, J=7.7, 1.8 Hz, 1H), 7.60 (m, 2H), 7.34 (m, 3H), 7.23 (m, 2H), 6.61 (dd, J=3.6, 0.7 Hz, 1H), 4.77 (d, J=4.6 Hz, 2H).

¹³C NMR (126 MHz, CDCl₃) δ 156.1, 152.3, 148.9, 137.0, 135.4, 130.2, 124.22, 124.19, 122.6, 122.3, 122.1, 121.1, 114.6, 107.1, 45.4.

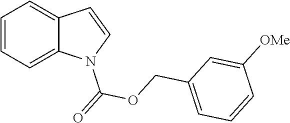 [SMILES: COc1cccc(COC(=O)n2ccc3ccccc32)c1]

### 3-Methoxybenzyl 1H-indole-1-carboxylate (MXC046)

¹H NMR (500 MHz, CDCl₃) δ 8.19 (s, 1H), 7.64 (d, J=3.6 Hz, 1H), 7.57 (d, J=7.8 Hz, 1H), 7.33 (m, 2H), 7.24 (app. td, J=7.7, 1.0 Hz, 1H), 7.06 (d, J=7.6 Hz, 1H), 7.02 (m, 1H), 6.92 (dd, J=7.9, 2.2 Hz, 1H), 6.60 (d, J=3.7 Hz, 1H), 5.43 (s, 2H), 3.83 (s, 3H).

¹³C NMR (126 MHz, CDCl₃) δ 159.9, 150.9, 136.7, 135.3, 130.6, 129.9, 125.6, 124.6, 123.1, 121.1, 120.6, 115.2, 114.2, 113.9, 108.3, 68.6, 55.3.

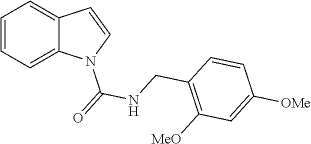 [SMILES: COc1ccc(CNC(=O)n2ccc3ccccc32)c(OC)c1]

### N-(2,4-Dimethoxybenzyl)-1H-indole-1-carboxamide (MXC047)

¹H NMR (500 MHz, CDCl₃) δ 8.00 (d, J=8.4 Hz, 1H), 7.58 (d, J=8.0 Hz, 1H), 7.46 (d, J=3.6 Hz, 1H), 7.28 (m, 2H), 7.20 (m, 1H), 6.58 (m, 1H), 6.48 (d, J=3.7 Hz, 1H), 6.18 (m, 1H), 4.57 (d, J=5.4 Hz, 2H), 3.87 (s, 3H), 3.80 (s, 3H).

¹³C NMR (126 MHz, CDCl₃) δ 160.8, 158.6, 151.9, 135.0, 130.8, 130.3, 124.5, 122.1, 121.2, 118.3, 113.8, 106.6, 104.1, 98.8, 55.5, 55.4, 40.8 (one low-field carbon signal is missing).

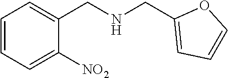 [SMILES: O=[N+]([O-])c1ccccc1CNCc1ccco1]

### 1-(Furan-2-yl)-N-(2-nitrobenzyl)methanamine (MXC048)

¹H NMR (500 MHz, CDCl₃) δ 7.96 (d, J=8.1 Hz, 1H), 7.63 (d, J=7.5 Hz, 1H), 7.59 (t, J=7.3 Hz, 1H), 7.42 (app. t, J=7.5 Hz, 1H), 7.37 (m, 1H), 6.32 (m, 1H), 6.20 (m, 1H), 4.05 (s, 2H), 3.82 (s, 2H).

¹³C NMR (126 MHz, CDCl₃) δ 153.4, 142.0, 135.2, 133.2, 131.4, 128.0, 124.8, 110.2, 107.3, 49.7, 45.6.

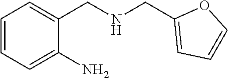 [SMILES: Nc1ccccc1CNCc1ccco1]

### 2-(((Furan-2-ylmethyl)amino)methyl)aniline (MXC049)

¹H NMR (500 MHz, CDCl₃) δ 7.37 (J=1.8, 0.7 Hz, 1H), 7.09 (dt, J=7.7, 1.5 Hz, 1H), 7.01 (dd, J=7.4, 1.1 Hz, 1H), 6.67 (m, 2H), 6.33 (dd, J=3.1, 1.9 Hz, 1H), 6.18 (d, J=2.6 Hz, 1H), 3.78 (s, 2H), 3.77 (s, 2H).

¹³C NMR (126 MHz, CDCl₃) δ 148.5, 146.6, 143.1, 131.7, 129.9, 125.5, 118.5, 117.1, 110.8, 110.6, 48.8, 43.0.

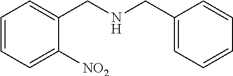 [SMILES: O=[N+]([O-])c1ccccc1CNCc1ccccc1]

### N-Benzyl-1-(2-nitrophenyl)methanamine (MXC050)

¹H NMR (500 MHz, CDCl₃) δ 7.94 (dd, J=8.1, 1.2 Hz, 1H), 7.63 (dd, J=7.7, 1.1 Hz, 1H), 7.58 (app. td, J=7.5, 1.3 Hz, 1H), 7.41 (m, 1H), 7.34 (m, 4H), 7.26 (m, 1H), 4.06 (s, 2H), 3.82 (s, 2H).

¹³C NMR (126 MHz, CDCl₃) δ 149.2, 140.0, 135.6, 133.1, 131.3, 128.5, 128.2, 128.0, 127.1, 124.8, 53.5, 50.2.

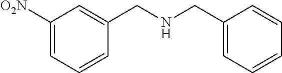 [SMILES: O=[N+]([O-])c1cccc(CNCc2ccccc2)c1]

### N-Benzyl-1-(3-nitrophenyl)methanamine (MXC051)

¹H NMR (500 MHz, CDCl₃) δ 8.24 (s, 1H), 8.10 (d, J=8.2 Hz, 1H), 7.70 (d, J=7.6 Hz, 1H), 7.48 (app. t, J=7.9 Hz, 1H), 7.35 (m, 4H), 7.28 (m, 1H), 3.90 (s, 2H), 3.82 (s, 2H).

¹³C NMR (126 MHz, CDCl₃) δ 148.4, 142.7, 139.8, 134.3, 129.3, 128.6, 128.2, 127.2, 122.9, 122.1, 53.3, 52.2.

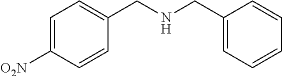 [SMILES: O=[N+]([O-])c1ccc(CNCc2ccccc2)cc1]

### N-Benzyl-1-(4-nitrophenyl)methanamine (MXC052)

¹H NMR (500 MHz, CDCl₃) δ 8.17 (d, J=8.8 Hz, 2H), 7.53 (d, J=8.8 Hz, 2H), 7.34 (m, 4H), 7.28 (m, 1H), 3.91 (s, 2H), 3.82 (s, 2H).

¹³C NMR (126 MHz, CDCl₃) δ 148.3, 147.0, 139.9, 128.7, 128.5, 128.1, 127.2, 123.6, 53.3, 52.3.

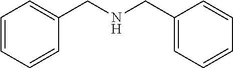 [SMILES: c1ccc(CNCc2ccccc2)cc1]

### Dibenzylamine (MXC053)

¹H NMR (500 MHz, CDCl₃) δ 7.37 (m, 8H), 7.30 (m, 2H), 3.84 (s, 4H).

¹³C NMR (126 MHz, CDCl₃) δ 140.6, 128.6, 128.4, 127.1, 53.4.

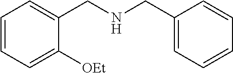 [SMILES: CCOc1ccccc1CNCc1ccccc1]

### N-Benzyl-1-(2-ethoxyphenyl)methanamine (MXC054)

¹H NMR (500 MHz, CDCl₃) δ 7.37 (m, 4H), 7.27 (m, 3H), 6.95 (app. td, J=7.4, 1.0 Hz, 1H), 6.88 (d, J=8.1 Hz, 1H), 4.07 (q, J=7.0 Hz, 2H), 3.86 (s, 2H), 3.82 (s, 2H), 1.44 (t, J=7.0 Hz, 3H).

¹³C NMR (126 MHz, CDCl₃) δ 157.2, 140.6, 130.2, 128.4, 128.3, 126.9, 120.4, 111.2, 63.5, 53.1, 49.0, 15.1 (two downfield C signals are missing).

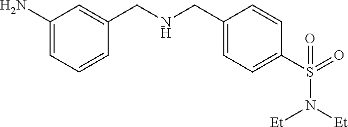 [SMILES: CCN(CC)S(=O)(=O)c1ccc(CNCc2cccc(N)c2)cc1]

### 4-(((3-Aminobenzyl)amino)methyl)-N,N-diethylbenzenesulfonamide (MXC055)

¹H NMR (500 MHz, CDCl₃) δ 8.24 (s, 1H), 8.11 (d, J=8.2 Hz, 1H), 7.77 (d, J=8.4 Hz, 2H), 7.68 (d, J=8.1 Hz, 1H), 7.51 (app. t, J=7.9 Hz, 1H), 7.48 (d, J=8.4 Hz, 2H), 3.91 (s, 2H), 3.88 (s, 2H), 3.23 (q, J=7.2 Hz, 4H), 1.12 (t, J=7.2 Hz, 6H).

¹³C NMR (126 MHz, CDCl₃) δ 148.4, 144.7, 142.3, 139.1, 134.2, 129.4, 128.5, 127.2, 122.9, 122.2, 52.6, 52.4, 42.1, 14.2.

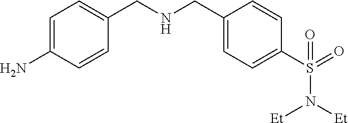 [SMILES: CCN(CC)S(=O)(=O)c1ccc(CNCc2ccc(N)cc2)cc1]

### 4-(((4-Aminobenzyl)amino)methyl)-N,N-diethylbenzenesulfonamide (MXC056)

¹H NMR (500 MHz, CDCl₃) δ 8.19 (d, J=8.8 Hz, 2H), 7.77 (d, J=8.4 Hz, 2H), 7.53 (d, J=8.8 Hz, 2H), 7.47 (d, J=8.8 Hz, 2H), 3.92 (s, 2H), 3.87 (s, 2H), 3.23 (q, J=7.2 Hz, 4H), 1.13 (t, J=7.2 Hz, 6H).

¹³C NMR (126 MHz, CDCl₃) δ 146.7, 145.3, 130.1, 128.6, 128.4, 127.8, 117.8, 115.8, 52.7, 52.6, 47.0, 25.2.

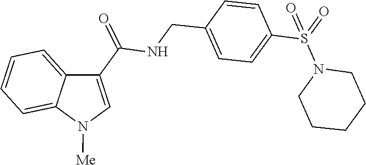 [SMILES: Cn1cc(C(=O)NCc2ccc(S(=O)(=O)N3CCCCC3)cc2)c2ccccc21]

### 1-Methyl-N-(4-(piperidin-1-ylsulfonyl)benzyl)-1H-indole-3-carboxamide (MXC057)

¹H NMR (500 MHz, CDCl₃) δ 7.99 (d, J=7.9 Hz, 1H), 7.72 (s, 1H), 7.64 (d, J=8.0 Hz, 2H), 7.48 (d, J=8.0 Hz, 2H), 7.37 (d, J=8.0 Hz, 1H), 7.30 (app. t, J=7.5 Hz, 1H), 7.26 (m, 1H), 6.54 (t, J=5.4 Hz, 1H), 4.75 (d, J=5.4 Hz, 1H), 3.82 (s, 3H), 2.94 (m, 4H), 1.61 (m, 4H), 1.41 (m, 2H).

¹³C NMR (126 MHz, CDCl₃) δ 165.3, 144.4, 137.3, 134.9, 132.5, 128.0, 127.8, 125.4, 122.7, 121.7, 120.3, 110.2, 110.1, 46.9, 42.7, 33.4, 25.2, 23.5.

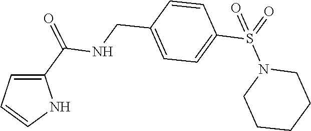 [SMILES: O=C(NCc1ccc(S(=O)(=O)N2CCCCC2)cc1)c1ccc[nH]1]

### N-(4-(Piperidin-1-ylsulfonyl)benzyl)-1H-pyrrole-2-carboxamide (MXC058)

¹H NMR (500 MHz, CDCl₃) δ 10.04 (s, 1H), 7.57 (d, J=8.3 Hz, 2H), 7.38 (d, J=8.2 Hz, 2H), 6.96 (s, 1H), 6.92 (m, 1H), 6.74 (m, 1H), 6.22 (m, 1H), 4.64 (br s, 2H), 2.92 (m, 4H), 1.61 (m, 4H), 1.40 (m, 2H)

¹³C NMR (126 MHz, CDCl₃) δ 161.4, 144.0, 134.8, 127.9, 127.8, 125.4, 122.0, 110.0, 109.7, 46.9, 42.6, 25.2, 23.4.

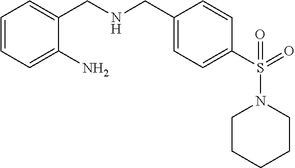 [SMILES: Nc1ccccc1CNCc1ccc(S(=O)(=O)N2CCCCC2)cc1]

### 2-(((4-(Piperidin-1-ylsulfonyl)benzyl)amino)methyl)aniline (MXC059)

¹H NMR (500 MHz, CDCl₃) δ 7.69 (d, J=8.0 Hz, 2H), 7.46 (d, J=8.0 Hz, 2H), 7.09 (app. t, J=7.4 Hz, 1H), 7.01 (d, J=7.1 Hz, 1H), 6.67 (m, 2H), 3.85 (s, 2H), 3.84 (s, 2H), 2.96 (m, 4H), 1.63 (m, 4H), 1.40 (m, 2H).

¹³C NMR (126 MHz, CDCl₃) δ 147.5, 147.2, 144.4, 139.3, 128.7, 128.5, 127.3, 123.7, 52.6, 52.4, 42.1, 29.7, 14.2 (two low-field carbon signals are missing).

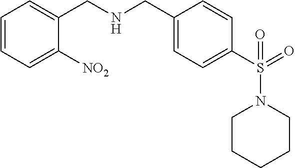 [SMILES: O=[N+]([O-])c1ccccc1CNCc1ccc(S(=O)(=O)N2CCCCC2)cc1]

### N-(2-Nitrobenzyl)-1-(4-(piperidin-1-ylsulfonyl)phenyl)methanamine (MXC060)

¹H NMR (500 MHz, CDCl₃) δ 7.94 (d, J=8.0 Hz, 1H), 7.69 (d, J=8.4 Hz, 2H), 7.58 (m, 2H), 7.49 (d, J=8.4 Hz, 2H), 7.43 (m, 1H), 4.06 (s, 2H), 3.88 (s, 2H), 2.96 (m, 4H), 1.62 (m, 4H), 1.40 (m, 2H).

¹³C NMR (126 MHz, CDCl₃) δ 149.3, 145.1, 135.0, 134.8, 133.2, 131.4, 128.5, 128.3, 127.8, 124.8, 52.9, 50.5, 47.0, 25.2, 23.5.

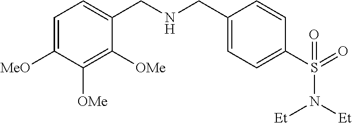 [SMILES: CCN(CC)S(=O)(=O)c1ccc(CNCc2ccc(OC)c(OC)c2OC)cc1]

### N,N-Diethyl-4-(((2,3,4-trimethoxybenzyl)amino)methyl)benzenesulfonamide (MXC061)

¹H NMR (500 MHz, CDCl₃) δ 7.75 (d, J=8.3 Hz, 2H), 7.47 (d, J=8.3 Hz, 2H), 6.91 (d, J=8.5 Hz, 1H), 6.62 (d, J=8.5 Hz, 1H), 3.89 (s, 3H), 3.87 (s, 3H), 3.85 (s, 3H), 3.83 (s, 2H), 3.72 (s, 2H), 3.22 (q, J=7.2 Hz, 4H), 1.12 (t, J=7.2 Hz, 6H).

¹³C NMR (126 MHz, CDCl₃) δ 153.1, 152.0, 145.5, 142.2, 138.6, 128.5, 127.0, 125.7, 124.2, 107.0, 61.0, 60.7, 56.0, 52.4, 48.4, 42.1, 14.2.

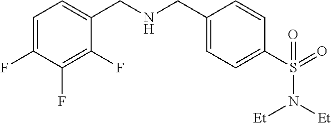 [SMILES: CCN(CC)S(=O)(=O)c1ccc(CNCc2ccc(F)c(F)c2F)cc1]

### N,N-Diethyl-4-(((2,3,4-trifluorobenzyl)amino)methyl)benzenesulfonamide (MXC062)

¹H NMR (500 MHz, CDCl₃) δ 7.76 (d, J=8.4 Hz, 2H), 7.47 (d, J=8.4 Hz, 2H), 7.08 (m, 1H), 6.94 (m, 1H), 3.85 (s, 2H), 3.84 (s, 2H), 3.23 (q, J=7.2 Hz, 4H), 1.12 (t, J=7.2 Hz, 6H).

¹³C NMR (126 MHz, CDCl₃) δ 150.3 (ddd, J_{CF}=249 Hz, J_{C-CF}=10 Hz, J_{C-C-CF}=3.1 Hz), 149.7 (ddd, J_{CF}=249 Hz, J_{C-CF}=9.9 Hz, J_{C-C-CF}=3.3 Hz), 144.7, 139.8 (dt, J_{CF}=252 Hz, J_{C-CF}=20 Hz, J_{C-C-CF}=12 Hz), 138.9, 128.5, 127.1, 124.6 (dd, J_{C-CF}=12.3 Hz, J_{C-C-CF}=2.6 Hz), 123.6 (td, J_{C-CF}=8.1 Hz, J_{C-C-CF}=4.7 Hz), 111.8 (dd, J_{C-CF}=17.2 Hz, J_{C-C-CF}=3.8 Hz), 52.3, 45.9, 42.0, 14.1.

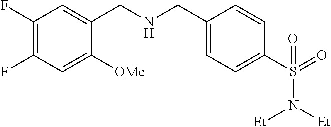 [SMILES: CCN(CC)S(=O)(=O)c1ccc(CNCc2cc(F)c(F)cc2OC)cc1]

### 4-(((4,5-Difluoro-2-methoxybenzyl)amino)methyl)-N,N-diethylbenzenesulfonamide (MXC063)

¹H NMR (500 MHz, CDCl₃) δ 7.76 (d, J=8.1 Hz, 2H), 7.46 (d, J=8.1 Hz, 2H), 7.10 (m, 1H), 6.68 (dd, J=12.0, 6.5 Hz, 1H), 3.82 (s, 2H), 3.79 (s, 3H), 3.72 (s, 2H), 3.23 (q, J=7.2 Hz, 4H), 1.13 (t, J=7.2 Hz, 6H).

¹³C NMR (126 MHz, CDCl₃) δ 153.6 (dd, J_{C-CF}=7.3 Hz, J_{C-C-CF}=2.0 Hz), 149.2 (dd, J_{CF}=246.1 Hz, J_{C-CF}=13.6 Hz), 145.2, 144.0 (dd, J_{CF}=239.8 Hz, J_{C-CF}=12.5 Hz) 138.8, 128.5, 127.1, 124.2 (t, J_{C-C-CF}=4.0 Hz), 117.8 (d, J_{C-CF}=18.6 Hz), 100.4 (d, J_{C-CF}=21.2 Hz), 56.0, 52.4, 47.5, 42.1, 14.2.

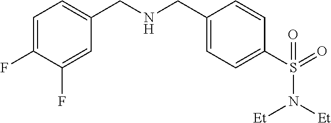 [SMILES: CCN(CC)S(=O)(=O)c1ccc(CNCc2ccc(F)c(F)c2)cc1]

### 4-(((3,4-Difluorobenzyl)amino)methyl)-N,N-diethylbenzenesulfonamide (MXC064)

¹H NMR (500 MHz, CDCl₃) δ 7.76 (d, J=8.4 Hz, 2H), 7.46 (d, J=8.4 Hz, 2H), 7.19 (ddd, J=11.1, 7.7, 2.0 Hz, 1H), 7.10 (m, 1H), 7.03 (m, 1H), 3.84 (s, 2H), 3.76 (s, 1H), 3.24 (q, J=7.2 Hz, 4H), 1.12 (t, J=7.2 Hz, 6H).

¹³C NMR (126 MHz, CDCl₃) δ 150.3 (dd, J_{CF}=247.8 Hz, J_{C-CF}=12.7 Hz), 149.4 (dd, J_{CF}=246.8 Hz, J_{C-CF}=12.7 Hz), 144.9, 138.9, 137.2 (dd, J_{C-CF}=4.9 Hz, J_{C-C-CF}=3.9 Hz), 128.5, 127.1, 123.9 (dd, J_{C-CF}=6.2 Hz, J_{C-C-CF}=3.5 Hz), 117.0 (d, J_{C-CF}=17.1 Hz), 116.8 (d, J_{C-CF}=17.1 Hz), 52.5, 52.2, 42.1, 14.2.

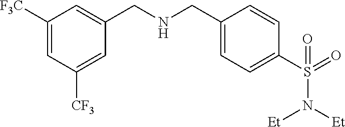 [SMILES: CCN(CC)S(=O)(=O)c1ccc(CNCc2cc(C(F)(F)F)cc(C(F)(F)F)c2)cc1]

### 4-(((3,5-Bis(trifluoromethyl)benzyl)amino)methyl)-N,N-diethylbenzenesulfonamide (MXC065)

¹H NMR (500 MHz, CDCl₃) δ 7.84 (d, J=7.5 Hz, 2H), 7.79 (s, 1H), 7.78 (s, 2H), 7.49 (d, J=7.5 Hz, 2H), 3.94 (s, 2H), 3.89 (s, 2H), 3.23 (q, J=7.2 Hz, 4H), 1.13 (t, J=7.2 Hz, 6H).

¹³C NMR (126 MHz, CDCl₃) δ 144.5, 142.8, 139.1, 131.6 (q, J=33.2 Hz), 128.6, 128.2, 127.2, 123.4 (q, J=272.6 Hz), 121.0, 52.7, 52.3, 42.1, 14.1.

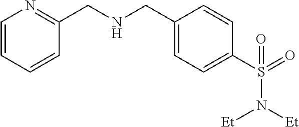 [SMILES: CCN(CC)S(=O)(=O)c1ccc(CNCc2ccccn2)cc1]

### N,N-Diethyl-4-(((pyridin-2-ylmethyl)amino)methyl)benzenesulfonamide (MXC066)

¹H NMR (500 MHz, CDCl₃) δ 8.53 (t, J=10.2 Hz, 1H), 7.74 (d, J=8.4 Hz, 2H), 7.64 (app. dt, J=7.7, 1.8 Hz, 1H), 7.48 (d, J=8.4 Hz, 2H), 7.28 (d, J=7.8 Hz, 1H), 7.17 (m, 1H), 3.91 (s, 2H), 3.88 (s, 2H), 3.21 (q, J=7.2 Hz, 4H), 1.11 (t, J=7.2 Hz, 6H).

¹³C NMR (126 MHz, CDCl₃) δ 159.2, 149.3, 144.9, 138.8, 136.6, 128.7, 127.1, 122.4, 122.1, 54.4, 52.8, 42.1, 14.2.

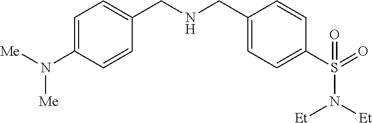 [SMILES: CCN(CC)S(=O)(=O)c1ccc(CNCc2ccc(N(C)C)cc2)cc1]

### 4-(((4-(Dimethylamino)benzyl)amino)methyl)-N,N-diethylbenzenesulfonamide (MXC067)

¹H NMR (500 MHz, CDCl₃) δ 7.75 (d, J=8.4 Hz, 2H), 7.46 (d, J=8.4 Hz, 2H), 7.19 (d, J=8.7 Hz, 2H), 6.71 (d, J=8.7 Hz, 2H), 3.84 (s, 2H), 3.70 (s, 2H), 3.22 (q, J=7.2 Hz, 4H), 2.93 (s, 6H), 1.12 (t, J=7.2 Hz, 6H).

¹³C NMR (126 MHz, CDCl₃) δ 149.9, 145.5, 138.7, 129.2, 128.6, 127.7, 127.1, 112.7, 52.8, 52.3, 42.1, 40.8, 14.2.

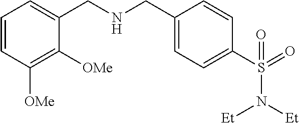 [SMILES: CCN(CC)S(=O)(=O)c1ccc(CNCc2cccc(OC)c2OC)cc1]

### 4-(((2,3-Dimethoxybenzyl)amino)methyl)-N,N-diethylbenzenesulfonamide (MXC068)

¹H NMR (500 MHz, CDCl₃) δ 7.73 (d, J=8.4 Hz, 2H), 7.46 (d, J=8.4 Hz, 2H), 7.01 (app. t, J=7.9 Hz, 1H), 6.85 (m, 1H), 3.86 (s, 3H), 3.81 (app. s, 5H), 3.79 (s, 2H), 3.21 (q, J=7.2 Hz, 2H), 1.10 (t, J=7.2 Hz, 6H).

¹³C NMR (126 MHz, CDCl₃) δ 152.7, 147.4, 145.4, 138.7, 133.5, 128.6, 127.0, 124.0, 121.8, 111.6, 60.7, 55.8, 52.3, 48.3, 42.1, 14.2.

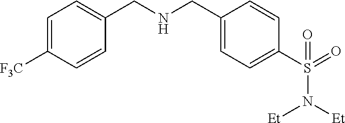 [SMILES: CCN(CC)S(=O)(=O)c1ccc(CNCc2ccc(C(F)(F)F)cc2)cc1]

### N,N-Diethyl-4-(((4-(trifluoromethyl)benzyl)amino)methyl)benzenesulfonamide (MXC069)

¹H NMR (500 MHz, CDCl₃) δ 7.75 (d, J=8.4 Hz, 2H), 7.58 (d, J=8.4 Hz, 2H), 7.47 (m, 4H), 3.86 (s, 2H), 3.82 (s, 2H), 3.22 (q, J=7.2 Hz, 4H), 1.12 (t, J=7.2 Hz, 6H).

¹³C NMR (126 MHz, CDCl₃) δ 144.9, 144.1, 138.9, 129.3 (q, J=32.3 Hz), 128.6, 128.4, 127.1, 125.3, 124.3 (q, J=272.6 Hz), 52.7, 52.5, 42.1, 14.2.

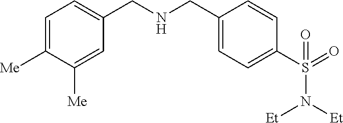 [SMILES: CCN(CC)S(=O)(=O)c1ccc(CNCc2ccc(C)c(C)c2)cc1]

### 4-(((3,4-Dimethylbenzyl)amino)methyl)-N,N-diethylbenzenesulfonamide (MXC070)

¹H NMR (500 MHz, CDCl₃) δ 7.75 (d, J=8.4 Hz, 2H), 7.47 (d, J=8.4 Hz, 2H), 7.10 (m, 3H), 7.05 (d, J=7.6 Hz, 1H), 3.85 (s, 2H), 3.73 (s, 2H), 3.23 (q, J=7.2 Hz, 4H), 2.26 (s, 3H), 2.25 (s, 3H), 1.12 (t, J=7.2 Hz, 6H).

¹³C NMR (126 MHz, CDCl₃) δ 145.3, 138.8, 137.3, 136.6, 135.4, 129.7, 129.6, 128.6, 127.1, 125.6, 53.0, 52.5, 42.1, 19.8, 19.5, 14.2.

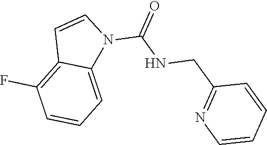 [SMILES: O=C(NCc1ccccn1)n1ccc2c(F)cccc21]

### 4-Fluoro-N-(pyridin-2-ylmethyl)-1H-indole-1-carboxamide (MXC071)

¹H NMR (500 MHz, CDCl₃) δ 8.59 (d, J=4.4 Hz, 1H), 8.00 (d, J=8.4 Hz, 1H), 7.74 (app. dt, J=7.7, 1.8 Hz, 1H), 7.58 (d, J=3.7 Hz, 1H), 7.37 (d, J=7.8 Hz, 1H), 7.27 (m, 2H), 6.91 (m, 2H), 6.74 (m, 1H), 4.79 (d, J=4.5 Hz, 2H).

¹³C NMR (126 MHz, CDCl₃) δ 155.9 (d, J=247.7 Hz), 155.4, 151.9, 148.6, 137.5 (d, J=9.9 Hz), 137.4, 124.9 (d, J=7.5 Hz), 124.2, 122.8, 122.4, 119.1 (d, J=22.3 Hz), 110.6 (d, J=3.7 Hz), 107.4 (d, J=18.5 Hz), 102.7, 45.1.

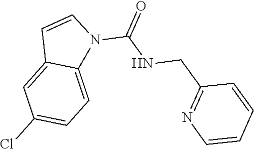 [SMILES: O=C(NCc1ccccn1)n1ccc2cc(Cl)ccc21]

### 5-Chloro-N-(pyridin-2-ylmethyl)-1H-indole-1-carboxamide (MXC072)

¹H NMR (500 MHz, CDCl₃) δ 8.58 (d, J=4.9 Hz, 1H), 8.18 (d, J=8.9 Hz, 1H), 7.74 (app. dt, J=7.7, 1.7 Hz, 1H), 7.60 (d, J=3.6 Hz, 1H), 7.56 (d, J=2.0 Hz, 1H), 7.36 (d, J=7.8 Hz, 1H), 7.33 (m, 1H), 7.28 (m, 2H), 6.58 (dd, J=3.6, 0.6 Hz, 1H), 4.77 (d, J=4.4 Hz, 2H).

¹³C NMR (126 MHz, CDCl₃) δ 155.1, 151.8, 147.6, 138.4, 133.8, 131.3, 127.9, 125.4, 124.4, 123.2, 123.0, 120.5, 115.7, 106.7, 44.6.

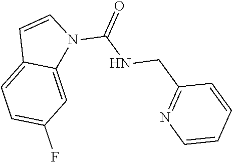 [SMILES: O=C(NCc1ccccn1)n1ccc2ccc(F)cc21]

### 6-Fluoro-N-(pyridin-2-ylmethyl)-1H-indole-1-carboxamide (MXC073)

¹H NMR (500 MHz, CDCl₃) δ 8.58 (d, J=4.3 Hz, 1H), 8.02 (dd, J=10.5, 2.3 Hz, 1H), 7.73 (app. dt, J=7.7, 1.8 Hz, 1H), 7.54 (d, J=3.7 Hz, 1H), 7.49 (dd, J=8.6, 5.4 Hz, 1H), 7.36 (d, J=7.8 Hz, 1H), 7.29 (m, 1H), 7.26 (m, 1H), 6.98 (m, 1H), 6.60 (dd, J=3.7, 0.7 Hz, 1H), 4.77 (d, J=4.5 Hz, 2H).

¹³C NMR (126 MHz, CDCl₃) δ 160.9 (d, J_{CF}=239.6 Hz), 155.3, 151.9, 148.0, 138.0, 135.7, 135.6, 126.3, 124.4 (d, J=3.9 Hz), 122.8 (d, J_{C-CF}=38.1 Hz), 121.5 (d, J_{C-C-CF}=10.0 Hz), 110.7 (d, J_{C-CF}=24.4 Hz), 107.2, 102.0 (d, J_{C-CF}=28.6 Hz), 44.8.

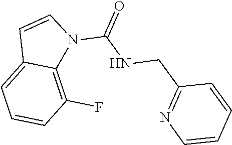 [SMILES: O=C(NCc1ccccn1)n1ccc2cccc(F)c21]

### 7-Fluoro-N-(pyridin-2-ylmethyl)-1H-indole-1-carboxamide (MXC074)

¹H NMR (500 MHz, CDCl₃) δ 8.58 (d, J=4.3 Hz, 1H), 7.92 (d, J=3.6 Hz, 1H), 7.76 (m, 1H), 7.70 (app. dt, J=7.7, 1.8 Hz, 1H), 7.40 (d, J=7.8 Hz, 1H), 7.37 (d, J=7.8 Hz, 1H), 7.23 (dd, J=7.2, 5.1 Hz, 1H), 7.16 (app. dt, J=7.9, 4.5 Hz, 1H), 7.04 (ddd, J=14.2, 8.0, 0.7 Hz, 1H), 6.63 (dd, J=3.6, 2.5 Hz, 1H), 4.81 (d, J=4.8 Hz, 2H).

¹³C NMR (126 MHz, CDCl₃) δ 155.5, 151.2, 149.3 (d, J=243.4 Hz), 147.6, 138.6, 135.4, 135.3, 128.8, 123.2, 123.0 (d, J=10.4 Hz), 120.8 (d, J=8.6 Hz), 117.6 (d, J=3.3 Hz), 110.5 (d, J=22.8 Hz), 106.5 (d, J=2.0 Hz), 45.6.

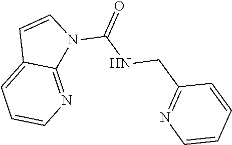 [SMILES: O=C(NCc1ccccn1)n1ccc2cccnc21]

### N-(Pyridin-2-ylmethyl)-1H-pyrrolo[2,3-b]pyridine-1-carboxamide (MXC075)

¹H NMR (500 MHz, CDCl₃) δ 10.46 (s, 1H), 8.61 (ddd, J=4.9, 1.7, 0.9 Hz, 1H), 8.32 (dd, J=4.9, 1.5 Hz, 1H), 8.02 (d, J=4.0 Hz, 1H), 7.95 (dd, J=7.8, 1.6 Hz, 1H), 7.69 (app. dt, J=7.7, 1.8 Hz, 1H), 7.45 (d, J=7.9 Hz, 1H), 7.21 (m, 2H), 6.55 (d, J=4.0 Hz, 1H), 4.89 (d, J=5.8 Hz, 2H).

¹³C NMR (126 MHz, CDCl₃) δ 157.3, 151.9, 148.3, 146.7, 142.6, 137.9, 129.9, 126.2, 123.5, 122.7, 122.4, 118.1, 103.3, 45.3.

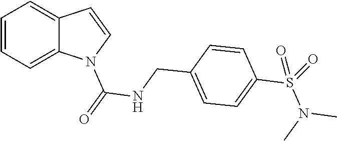 [SMILES: CN(C)S(=O)(=O)c1ccc(CNC(=O)n2ccc3ccccc32)cc1]

### N-(4-(N,N-dimethylsulfamoyl)benzyl)-1H-indole-1-carboxamide (MXC076)

¹H NMR (500 MHz, CDCl₃) δ 8.19 (d, J=8.4 Hz, 1H), 7.59 (d, J=7.7 Hz, 1H), 7.55 (m, 3H), 7.42 (d, J=8.3 Hz, 2H), 7.30 (m, 1H), 7.22 (m, 1H), 6.64 (m, 2H), 4.68 (d, J=5.9 Hz, 2H), 2.64 (s, 6H).

¹³C NMR (126 MHz, CDCl₃) δ 152.4, 143.6, 135.4, 134.2, 130.2, 128.0, 127.9, 124.4, 123.8, 122.6, 121.2, 114.6, 107.7, 44.0, 37.9.

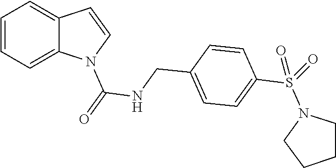 [SMILES: O=C(NCc1ccc(S(=O)(=O)N2CCCC2)cc1)n1ccc2ccccc21]

### N-(4-(Pyrrolidin-1-ylsulfonyl)benzyl)-1H-indole-1-carboxamide (MXC077)

¹H NMR (500 MHz, CDCl₃) δ 8.20 (d, J=8.3 Hz, 1H), 7.58 (m, 4H), 7.39 (d, J=8.3 Hz, 2H), 7.30 (m, 1H), 7.22 (m, 1H), 6.71 (app. t, J=5.6 Hz, 1H), 6.63 (d, J=3.7 Hz, 1H), 4.68 (d, J=5.9 Hz, 2H), 3.16 (m, 4H), 1.72 (m, 4H).

¹³C NMR (126 MHz, CDCl₃) δ 152.4, 143.4, 135.6, 135.4, 130.2, 127.8, 127.7, 124.4, 123.8, 122.5, 121.2, 114.6, 107.6, 47.9, 44.0, 25.2.

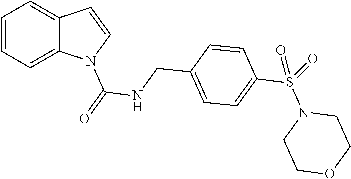 [SMILES: O=C(NCc1ccc(S(=O)(=O)N2CCOCC2)cc1)n1ccc2ccccc21]

### N-(4-(Morpholinosulfonyl)benzyl)-1H-indole-1-carboxamide (MXC078)

¹H NMR (500 MHz, DMSO-d₆) δ 8.83 (t, J=5.8 Hz, 1H), 8.23 (d, J=8.3 Hz, 1H), 7.90 (d, J=3.7 Hz, 1H), 7.71 (d, J=8.4 Hz, 2H), 7.64 (d, J=8.4 Hz, 2H), 7.58 (d, J=7.7 Hz, 1H), 7.24 (m, 1H), 7.16 (m, 1H), 6.70 (d, J=3.7 Hz, 1H), 4.60 (d, J=5.8 Hz, 2H), 3.59 (m, 4H), 2.82 (m, 4H).

¹³C NMR (126 MHz, CDCl₃) δ 152.5, 145.7, 135.9, 133.4, 129.9, 128.44, 128.41, 125.2, 124.4, 122.5, 121.2, 115.5, 107.0, 65.7, 46.4, 43.4.

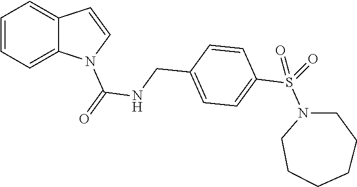 [SMILES: O=C(NCc1ccc(S(=O)(=O)N2CCCCCC2)cc1)n1ccc2ccccc21]

### N-(4-(Azepan-1-ylsulfonyl)benzyl)-1H-indole-1-carboxamide (MXC079)

¹H NMR (500 MHz, CDCl₃) δ 8.20 (d, J=8.4 Hz, 1H), 7.56 (m, 4H), 7.37 (d, J=8.2 Hz, 2H), 7.30 (m, 1H), 7.22 (app. t, J=7.5 Hz, 1H), 6.68 (m, 1H), 6.62 (d, J=3.6 Hz, 1H), 4.66 (d, J=5.8 Hz, 2H), 3.19 (m, 4H), 1.67 (m, 4H), 1.56 (m, 4H).

¹³C NMR (126 MHz, CDCl₃) δ 152.6, 143.1, 138.1, 135.5, 130.1, 127.9, 127.1, 124.3, 123.9, 122.5, 121.1, 114.7, 107.5, 48.2, 43.9, 29.1, 26.9.

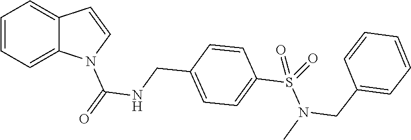 [SMILES: CN(Cc1ccccc1)S(=O)(=O)c1ccc(CNC(=O)n2ccc3ccccc32)cc1]

### N-(4-(N-Benzyl-N-methylsulfamoyl)benzyl)-1H-indole-1-carboxamide (MXC080)

¹H NMR (500 MHz, CDCl₃) δ 8.15 (d, J=8.3 Hz, 1H), 7.75 (d, J=8.0 Hz, 2H), 7.61 (d, J=7.8 Hz, 1H), 7.53 (m, 3H), 7.32 (m, 6H), 7.23 (d, J=7.8 Hz, 1H), 6.65 (d, J=3.6 Hz, 1H) 6.25 (m, 1H), 4.76 (d, J=5.8 Hz, 2H), 4.11 (s, 2H), 2.58 (s, 3H).

¹³C NMR (126 MHz, CDCl₃) δ 152.3, 143.3, 136.5, 135.4, 135.3, 130.2, 128.7, 128.4, 128.2, 128.0, 127.9, 124.5, 123.8, 122.6, 121.3, 114.3, 107.7, 54.1, 44.2, 34.4.

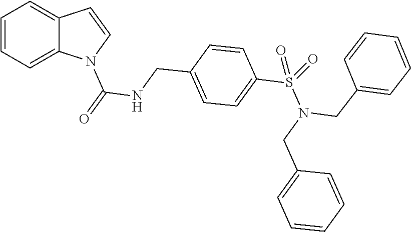 [SMILES: O=C(NCc1ccc(S(=O)(=O)N(Cc2ccccc2)Cc2ccccc2)cc1)n1ccc2ccccc21]

### N-(4-(N,N-Dibenzylsulfamoyl)benzyl)-1H-indole-1-carboxamide (MXC081)

¹H NMR (500 MHz, CDCl₃) δ 8.16 (d, J=8.4 Hz, 1H), 7.70 (d, J=8.2 Hz, 2H), 7.60 (d, J=7.8 Hz, 1H), 7.52 (d, J=3.7 Hz, 1H), 7.46 (d, J=8.2 Hz, 2H), 7.33 (m, 1H), 7.23 (d, J=7.1 Hz, 1H), 7.20 (m, 6H), 7.04 (m, 4H), 6.63 (d, J=3.6 Hz, 1H), 6.32 (app. s, 1H), 4.73 (d, J=5.8 Hz, 2H), 4.29 (s, 4H).

¹³C NMR (126 MHz, CDCl₃) δ 152.3, 143.2, 139.6, 135.4, 135.3, 130.2, 128.54, 128.50, 128.1, 127.8, 127.6, 124.5, 123.4, 122.6, 121.3, 114.4, 107.7, 50.7, 44.2.

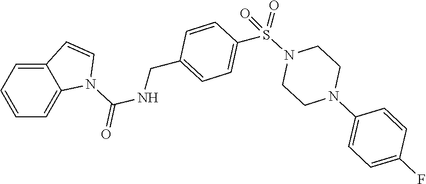 [SMILES: O=C(NCc1ccc(S(=O)(=O)N2CCN(c3ccc(F)cc3)CC2)cc1)n1ccc2ccccc21]

### N-(4-((4-(4-fluorophenyl)piperazin-1-yl)sulfonyl)benzyl)-1H-indole-1-carboxamide (MXC082)

¹H NMR (500 MHz, CDCl₃) δ 8.17 (dd, J=8.3, 0.6 Hz, 1H), 7.62 (d, J=8.2 Hz, 2H), 7.59 (d, J=7.8 Hz, 1H), 7.53 (d, J=3.7 Hz, 1H), 7.47 (d, J=8.2 Hz, 2H), 7.30 (app. t, J=7.8 Hz, 1H), 7.22 (app. t, J=7.5 Hz, 1H), 6.94 (m, 2H), 6.82 (m, 2H), 6.63 (d, J=3.7 Hz, 1H), 6.54 (m, 1H), 4.68 (d, J=5.9 Hz, 2H), 3.11 (m, 8H).

¹³C NMR (126 MHz, CDCl₃) δ 157.9 (d, J=242.0 Hz), 152.4, 147.0 (d, J=10 Hz), 143.9, 135.4, 134.2, 130.2, 128.2, 124.5, 123.8, 122.6, 121.3, 119.1 (d, J=12.3 Hz), 115.8 (d, J=22 Hz), 116.4 (d, J=20 Hz), 114.5 (d, J=4.2 Hz), 107.7, 50.2, 45.9, 44.1.

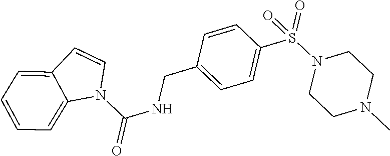 [SMILES: CN1CCN(S(=O)(=O)c2ccc(CNC(=O)n3ccc4ccccc43)cc2)CC1]

### N-(4-((4-Methylpiperazin-1-yl)sulfonyl)benzyl)-1H-indole-1-carboxamide (MXC083)

¹H NMR (500 MHz, CDCl₃) δ 8.13 (dd, J=8.4, 0.7 Hz, 1H), 7.71 (d, J=8.4 Hz, 2H), 7.60 (d, J=7.8 Hz, 1H), 7.54 (d, J=8.5 Hz, 2H), 7.48 (d, J=3.7 Hz, 1H), 7.33 (dd, J=8.3, 7.2 Hz, 1H), 7.24 (m, 1H), 6.65 (m, 1H), 6.65 (dd, J=3.7, 0.7 Hz, 1H), 6.09 (t, J=5.9 Hz, 1H), 4.72 (d, J=5.9 Hz, 2H), 3.06 (m, 4H), 2.52 (m, 4H), 2.29 (s, 3H).

¹³C NMR (126 MHz, CDCl₃) δ 152.2, 143.5, 135.3, 134.5, 130.2, 128.30, 128.25, 124.5, 123.8, 122.6, 121.3, 114.4, 107.7, 54.0, 45.8, 45.6, 44.2.

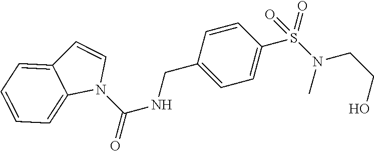 [SMILES: CN(CCO)S(=O)(=O)c1ccc(CNC(=O)n2ccc3ccccc32)cc1]

### N-(4-(N-(2-Hydroxyethyl)-N-methylsulfamoyl)benzyl)-1H-indole-1-carboxamide (MXC084)

¹H NMR (500 MHz, CDCl₃) δ 8.15 (dd, J=8.3, 0.7 Hz, 1H), 7.62 (d, J=8.4 Hz, 2H), 7.59 (d, J=7.8 Hz, 1H), 7.52 (d, J=3.7 Hz, 1H), 7.44 (d, J=8.4 Hz, 2H), 7.30 (app. t, J=7.8 Hz, 1H), 7.22 (app. t, J=7.5 Hz, 1H), 6.63 (dd, J=3.7, 0.6 Hz, 1H), 6.54 (t, J=5.4 Hz, 1H), 4.66 (d, J=5.4 Hz, 2H), 3.70 (t, J=5.3 Hz, 2H), 3.09 (t, J=5.3 Hz, 2H), 2.76 (s, 3H).

¹³C NMR (126 MHz, CDCl₃) δ 152.2, 143.5, 135.3, 134.5, 130.2, 128.30, 128.25, 124.5, 123.8, 122.6, 121.3, 114.4, 107.7, 54.0, 45.8, 45.6, 44.2.

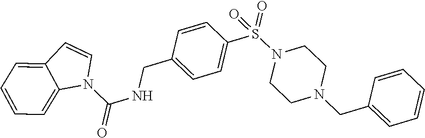 [SMILES: O=C(NCc1ccc(S(=O)(=O)N2CCN(Cc3ccccc3)CC2)cc1)n1ccc2ccccc21]

### N-(4-((4-Benzylpiperazin-1-yl)sulfonyl)benzyl)-1H-indole-1-carboxamide (MXC085)

¹H NMR (500 MHz, CDCl₃) δ 8.17 (d, J=8.3 Hz, 1H), 7.66 (d, J=8.3 Hz, 2H), 7.59 (d, J=7.7 Hz, 1H), 7.55 (d, J=3.7 Hz, 1H), 7.51 (d, J=8.4 Hz, 2H), 7.31 (m, 6H), 7.23 (m, 1H), 6.63 (dd, J=3.7, 0.6 Hz, 1H), 6.41 (br. s, 1H), 4.70 (d, J=5.8 Hz, 2H), 3.66 (m, 2H), 3.14 (m, 4H), 2.69 (m, 4H).

¹³C NMR (126 MHz, CDCl₃) δ 152.2, 143.4, 135.2, 134.6, 130.2, 129.4, 128.5, 128.4, 128.3, 127.6, 127.2, 124.5, 123.8, 122.6, 121.3, 114.3, 107.7, 64.0, 51.9, 45.8, 44.3.

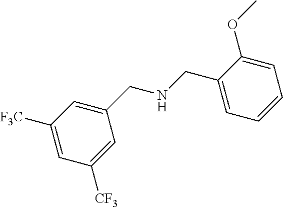 [SMILES: COc1ccccc1CNCc1cc(C(F)(F)F)cc(C(F)(F)F)c1]

### N-(3,5-Bis(trifluoromethyl)benzyl)-1-(2-methoxyphenyl)methanamine (MXC086)

¹H NMR (500 MHz, CDCl₃) δ 7.84 (s, 2H), 7.75 (s, 1H), 7.27 (m, 1H), 7.21 (dd, J=7.4, 1.6 Hz, 1H), 6.93 (app. dt, J=7.4, 1.0 Hz, 1H), 6.89 (d, J=8.2 Hz, 1H), 3.89 (s, 2H), 3.84 (s, 3H). 3.81 (s, 2H).

¹³C NMR (126 MHz, CDCl₃) δ 157.8, 148.5, 131.5, 130.1, 128.7, 127.7, 123.6 (q, J=33.0 Hz), 130.1, 128.7, 127.7, 123.6 (q, J=272.5 Hz), 120.8, 120.5, 110.4, 55.1, 51.9, 48.8.

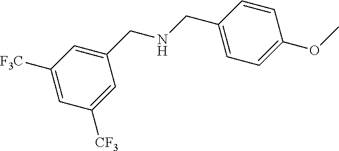 [SMILES: COc1ccc(CNCc2cc(C(F)(F)F)cc(C(F)(F)F)c2)cc1]

### N-(3,5-Bis(trifluoromethyl)benzyl)-1-(4-methoxyphenyl)methanamine (MXC087)

¹H NMR (500 MHz, CDCl₃) δ 7.83 (s, 2H), 7.76 (s, 1H), 7.25 (d, J=8.8 Hz, 2H), 6.88 (d, J=8.8 Hz, 2H), 3.91 (s, 2H), 3.81 (s, 3H), 3.76 (s, 2H).

¹³C NMR (126 MHz, CDCl₃) δ 158.9, 143.4, 131.8, 131.5 (q, J=33.1 Hz), 129.4, 128.2, 123.6 (q, J=272.5 Hz), 120.9, 113.9, 55.1, 52.8, 52.0.

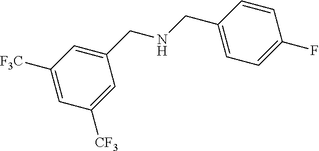 [SMILES: Fc1ccc(CNCc2cc(C(F)(F)F)cc(C(F)(F)F)c2)cc1]

### N-(3,5-Bis(trifluoromethyl)benzyl)-1-(4-fluorophenyl)methanamine (MXC088)

¹H NMR (500 MHz, CDCl₃) δ 7.82 (s, 2H), 7.77 (s, 1H), 7.30 (dd, J=8.7, 5.4 Hz, 2H), 7.02 (m, 2H), 3.91 (s, 2H), 3.79 (s, 2H).

¹³C NMR (126 MHz, CDCl₃) δ 162.1 (d, J=245.0 Hz), 143.1, 135.5, 131.6 (q, J=33.2 Hz), 129.7 (d, J=7.9 Hz), 128.2, 123.5 (q, J=272.5 Hz), 121.0, 115.3 (d, J=21.3 Hz), 52.6, 52.1.

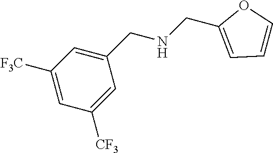 [SMILES: FC(F)(F)c1cc(CNCc2ccco2)cc(C(F)(F)F)c1]

### N-(3,5-Bis(trifluoromethyl)benzyl)-1-(furan-2-yl)methanamine (MXC089)

¹H NMR (500 MHz, CDCl₃) δ 7.81 (s, 2H), 7.76 (s, 1H), 7.37 (dd, J=1.8, 0.8 Hz, 1H), 6.32 (dd, J=3.2, 1.9 Hz, 1H), 6.18 (dd, J=3.2, 0.7 Hz, 1H), 3.90 (s, 2H), 3.81 (s, 2H).

¹³C NMR (126 MHz, CDCl₃) δ 153.2, 142.8, 142.1, 131.6 (q, J=33.2 Hz), 128.2, 123.5 (q, J=272.5 Hz), 120.9, 110.2, 107.4, 51.6, 45.3.

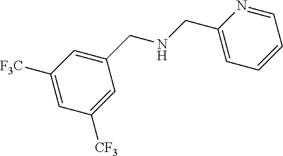 [SMILES: FC(F)(F)c1cc(CNCc2ccccn2)cc(C(F)(F)F)c1]

### N-(3,5-Bis(trifluoromethyl)benzyl)-1-(pyridin-2-yl)methanamine (MXC090)

¹H NMR (500 MHz, CDCl₃) δ 8.56 (ddd, J=4.8, 1.7, 0.9 Hz, 1H), 7.84 (s, 2H), 7.75 (s, 1H), 7.65 (app. dt, J=7.7, 1.8 Hz, 1H), 7.27 (d, J=7.8 Hz, 1H), 7.18 (m, 1H), 3.96 (s, 2H), 3.93 (s, 2H).

¹³C NMR (126 MHz, CDCl₃) δ 158.9, 149.4, 142.9, 136.5, 131.6 (q, J=33.2 Hz), 128.3, 123.5 (q, J=272.5 Hz), 122.4, 122.2, 120.9, 54.4, 52.4.

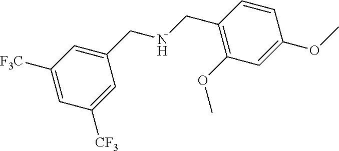 [SMILES: COc1ccc(CNCc2cc(C(F)(F)F)cc(C(F)(F)F)c2)c(OC)c1]

### N-(3,5-Bis(trifluoromethyl)benzyl)-1-(2,4-dimethoxyphenyl)methanamine (MXC091)

¹H NMR (500 MHz, CDCl₃) δ 7.82 (s, 2H), 7.74 (s, 1H), 7.09 (d, J=8.1 Hz, 1H), 6.45 (m, 2H), 3.87 (s, 2H), 3.81 (s, 3H), 3.80 (s, 3H), 3.73 (s, 2H).

¹³C NMR (126 MHz, CDCl₃) δ 160.4, 158.8, 143.6, 131.6 (q, J=33.2 Hz), 130.7, 128.2, 123.5 (q, J=272.5 Hz), 120.7, 120.2, 103.7, 98.6, 55.2, 55.1, 51.7, 48.3.

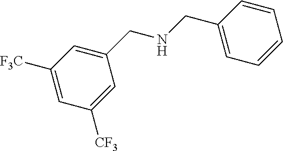 [SMILES: FC(F)(F)c1cc(CNCc2ccccc2)cc(C(F)(F)F)c1]

### N-Benzyl-1-(3,5-bis(trifluoromethyl)phenyl)methanamine (MXC092)

¹H NMR (500 MHz, CDCl₃) δ 7.84 (s, 2H), 7.76 (s, 1H), 7.35 (m, 4H), 7.28 (s, 1H), 3.92 (s, 2H), 3.83 (s, 2H).

¹³C NMR (126 MHz, CDCl₃) δ 143.2, 139.7, 131.6 (q, J=33.2 Hz), 128.6, 127.3, 123.5 (q, J=272.5 Hz), 121.0, 53.4, 52.1 (one low-field carbon signal missing).

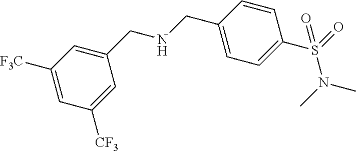 [SMILES: CN(C)S(=O)(=O)c1ccc(CNCc2cc(C(F)(F)F)cc(C(F)(F)F)c2)cc1]

### 4-(((3,5-Bis(trifluoromethyl)benzyl)amino)methyl)-N,N-dimethylbenzenesulfonamide (MXC093)

¹H NMR (500 MHz, CDCl₃) δ 7.84 (s, 2H), 7.77 (s, 1H), 7.75 (d, J=8.4 Hz, 2H), 7.53 (d, J=8.4 Hz, 2H), 3.95 (s, 2H), 3.91 (s, 2H), 2.70 (s, 6H).

¹³C NMR (126 MHz, CDCl₃) δ 145.1, 142.8, 134.2, 131.6 (q, J=33.2 Hz), 128.6, 128.2, 123.5 (q, J=272.5 Hz), 121.1, 52.8, 52.4, 37.9.

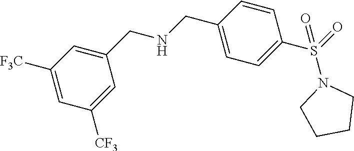 [SMILES: O=S(=O)(c1ccc(CNCc2cc(C(F)(F)F)cc(C(F)(F)F)c2)cc1)N1CCCC1]

### N-(3,5-Bis(trifluoromethyl)benzyl)-1-(4-(pyrrolidin-1-ylsulfonyl)phenyl)methanamine (MXC094)

¹H NMR (500 MHz, CDCl₃) δ 7.84 (s, 2H), 7.80 (d, J=8.4 Hz, 2H), 7.78 (s, 1H), 7.51 (d, J=8.4 Hz, 2H), 3.95 (s, 2H), 3.90 (s, 2H), 3.24 (m, 4H), 1.76 (m, 4H).

¹³C NMR (126 MHz, CDCl₃) δ 144.9, 142.8, 135.6, 131.6 (q, J=33.2 Hz), 128.5, 128.2, 127.7, 123.5 (q, J=272.5 Hz), 121.0, 52.7, 52.3, 47.9, 25.2.

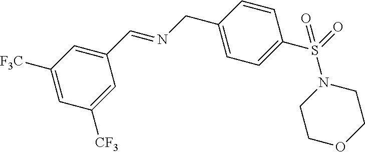 [SMILES: O=S(=O)(c1ccc(C/N=C/c2cc(C(F)(F)F)cc(C(F)(F)F)c2)cc1)N1CCOCC1]

### (E)-1-(3,5-Bis(trifluoromethyl)phenyl)-N-(4-(morpholinosulfonyl)benzyl)methanimine (MXC095)

¹H NMR (500 MHz, CDCl₃) δ 8.53 (s, 1H), 8.25 (s, 2H), 7.95 (s, 1H), 7.75 (d, J=8.4 Hz, 2H), 7.55 (d, J=8.4 Hz, 2H), 4.94 (s, 2H), 3.73 (m, 4H), 3.00 (m, 4H).

¹³C NMR (126 MHz, CDCl₃) δ 159.7, 144.2, 137.6, 134.0, 132.3 (q, J=33.8 Hz), 128.5, 128.3, 128.1, 124.4, 123.1 (q, J=272.8 Hz), 66.1, 64.2, 46.0.

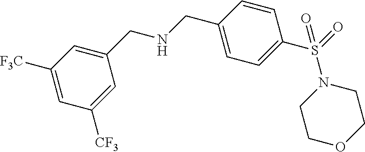 [SMILES: O=S(=O)(c1ccc(CNCc2cc(C(F)(F)F)cc(C(F)(F)F)c2)cc1)N1CCOCC1]

### N-(3,5-Bis(trifluoromethyl)benzyl)-1-(4-(morpholinosulfonyl)phenyl)methanamine (MXC096)

¹H NMR (500 MHz, CDCl₃) δ 7.86 (s, 2H), 7.79 (s, 1H), 7.73 (d, J=8.4 Hz, 2H), 7.56 (d, J=8.4 Hz, 2H), 3.96 (s, 2H), 3.92 (s, 2H), 3.74 (m, 4H), 2.99 (m, 4H).

¹³C NMR (126 MHz, CDCl₃) δ 145.4, 142.6, 133.9, 131.6 (q, J=33.2 Hz), 128.6, 128.3, 128.1, 123.5 (q, J=272.5 Hz), 121.2, 66.1, 52.7.

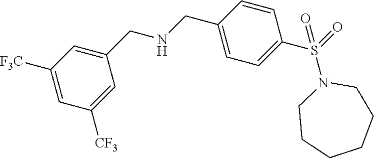 [SMILES: O=S(=O)(c1ccc(CNCc2cc(C(F)(F)F)cc(C(F)(F)F)c2)cc1)N1CCCCCC1]

### N-(4-(Azepan-1-ylsulfonyl)benzyl)-1-(3,5-bis(trifluoromethyl)phenyl)methanamine (MXC097)

¹H NMR (500 MHz, CDCl₃) δ 7.84 (s, 2H), 7.77 (s, 1H), 7.76 (d, J=8.4 Hz, 2H), 7.48 (d, J=8.4 Hz, 2H), 3.94 (s, 2H), 3.89 (s, 2H), 3.26 (m, 4H), 1.71 (m, 4H), 1.58 (m, 4H).

¹³C NMR (126 MHz, CDCl₃) δ 144.5, 142.9, 138.3, 131.6 (q, J=33.2 Hz), 128.5, 128.2, 127.1, 123.5 (q, J=272.5 Hz), 121.0, 52.7, 52.3, 48.2, 29.2, 26.9.

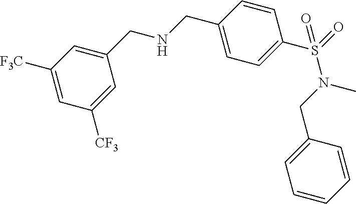 [SMILES: CN(Cc1ccccc1)S(=O)(=O)c1ccc(CNCc2cc(C(F)(F)F)cc(C(F)(F)F)c2)cc1]

### N-Benzyl-4-(((3,5-bis(trifluoromethyl)benzyl)amino)methyl)-N-methylbenzenesulfonamide (MXC098)

¹H NMR (500 MHz, CDCl₃) δ 7.86 (s, 2H), 7.82 (d, J=8.4 Hz, 2H), 7.79 (s, 1H), 7.55 (d, J=8.4 Hz, 2H), 7.32 (m, 5H), 4.15 (s, 2H), 3.97 (s, 2H), 3.93 (s, 2H), 2.60 (s, 3H).

¹³C NMR (126 MHz, CDCl₃) δ 145.1, 142.8, 136.1, 135.6, 131.6 (q, J=33.2 Hz), 128.71, 128.68, 128.4, 128.2, 128.0, 127.7, 123.5 (q, J=272.5 Hz), 121.1, 54.2, 52.8, 52.4, 34.4.

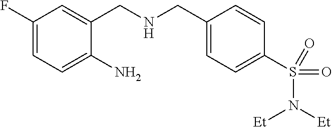 [SMILES: CCN(CC)S(=O)(=O)c1ccc(CNCc2cc(F)ccc2N)cc1]

### 4-(((2-Amino-5-fluorobenzyl)amino)methyl)-N,N-diethylbenzenesulfonamide (MXC099)

¹H NMR (500 MHz, CDCl₃) δ 7.76 (d, J=8.4 Hz, 2H), 7.44 (d, J=8.4 Hz, 2H), 6.79 (m, 2H), 6.59 (dd, J=8.6, 4.8 Hz, 1H), 3.85 (s, 2H), 3.80 (s, 2H), 3.23 (q, J=7.2 Hz, 4H), 1.12 (t, J=7.1 Hz, 6H).

¹³C NMR (126 MHz, CDCl₃) δ 155.8 (d, J=235.4 Hz), 144.7, 142.6 (d, J=2.0 Hz), 139.1, 128.6, 127.2, 124.7 (d, J=6.5 Hz), 116.5 (d, J=3.8 Hz), 116.4 (d, J=10.9 Hz), 114.7 (d, J=21.9 Hz), 52.7, 52.0, 42.1, 14.2.

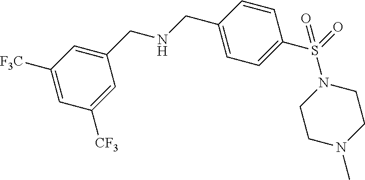 [SMILES: CN1CCN(S(=O)(=O)c2ccc(CNCc3cc(C(F)(F)F)cc(C(F)(F)F)c3)cc2)CC1]

### N-(3,5-Bis(trifluoromethyl)benzyl)-1-(4-((4-methylpiperazin-1-yl)sulfonyl)phenyl)methanamine (MXC101)

¹H NMR (500 MHz, CDCl₃) δ 7.84 (s, 2H), 7.78 (s, 1H), 7.71 (d, J=8.2 Hz, 2H), 7.51 (d, J=8.2 Hz, 2H), 3.93 (s, 2H), 3.89 (s, 2H), 3.05 (m, 4H), 2.53 (m, 4H), 2.29 (s, 3H).

¹³C NMR (126 MHz, CDCl₃) δ 145.3, 142.6, 134.1, 131.6 (q, J=33.2 Hz), 128.6, 128.2, 128.0, 123.5 (q, J=272.5 Hz), 121.1, 53.9, 52.7, 52.2, 45.7, 45.4.

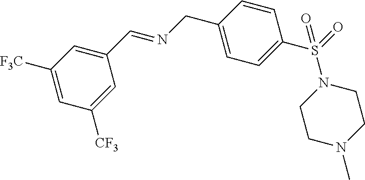 [SMILES: CN1CCN(S(=O)(=O)c2ccc(C/N=C/c3cc(C(F)(F)F)cc(C(F)(F)F)c3)cc2)CC1]

### (E)-1-(3,5-Bis(trifluoromethyl)phenyl)-N-(4-((4-methylpiperazin-1-yl)sulfonyl)benzyl)methanimine (MXC102)

¹H NMR (500 MHz, CDCl₃) δ 8.51 (s, 1H), 8.25 (s, 2H), 7.94 (s, 1H), 7.74 (d, J=8.2 Hz, 2H), 7.52 (d, J=8.2 Hz, 2H), 4.92 (s, 2H), 3.03 (m, 4H), 2.48 (m, 4H), 2.26 (s, 3H).

¹³C NMR (126 MHz, CDCl₃) δ 159.6, 143.9, 137.7, 134.1, 132.3 (q, J=33.7 Hz), 128.5, 128.2, 124.3, 123.1 (q, J=272.8 Hz), 64.2, 54.0, 45.8, 45.6 (one low-field carbon signal is missing).

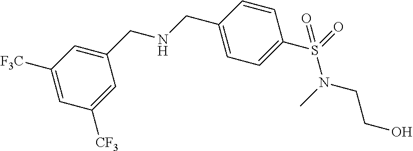 [SMILES: CN(CCO)S(=O)(=O)c1ccc(CNCc2cc(C(F)(F)F)cc(C(F)(F)F)c2)cc1]

### 4-(((3,5-Bis(trifluoromethyl)benzyl)amino)methyl)-N-(2-hydroxyethyl)-N-methylbenzenesulfonamide (MXC103)

¹H NMR (500 MHz, CDCl₃) δ 7.85 (s, 2H), 7.78 (m, 3H), 7.54 (d, J=8.2 Hz, 2H), 3.96 (s, 2H), 3.92 (s, 2H), 3.77 (t, J=5.2 Hz, 2H), 3.17 (t, J=5.2 Hz, 2H), 2.84 (s, 3H).

¹³C NMR (126 MHz, CDCl₃) δ 145.1, 142.6, 136.0, 132.3 (q, J=33.7 Hz), 128.8, 128.2, 127.6, 123.1 (q, J=272.8 Hz), 121.1, 60.2, 52.6, 52.5, 52.3, 36.0.

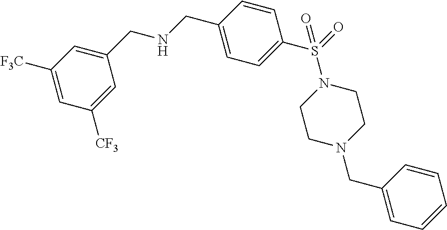 [SMILES: O=S(=O)(c1ccc(CNCc2cc(C(F)(F)F)cc(C(F)(F)F)c2)cc1)N1CCN(Cc2ccccc2)CC1]

### N-(4-((4-Benzylpiperazin-1-yl)sulfonyl)benzyl)-1-(3,5-bis(trifluoromethyl)phenyl)methanamine (MXC104)

¹H NMR (500 MHz, CDCl₃) δ 7.85 (s, 2H), 7.78 (s, 1H), 7.71 (d, J=8.3 Hz, 2H), 7.51 (d, J=8.3 Hz, 2H), 7.25 (m, 5H), 3.95 (s, 2H), 3.48 (s, 2H), 3.90 (s, 2H), 3.02 (m, 4H), 2.52 (m, 4H).

¹³C NMR (126 MHz, CDCl₃) δ 145.1, 142.6, 137.3, 134.4, 132.3 (q, J=33.7 Hz), 129.2, 128.6, 128.4, 128.2, 127.4, 123.1 (q, J=272.8 Hz), 121.2, 62.7, 52.7, 52.4, 52.1, 46.1 (one low-field carbon signal is missing).

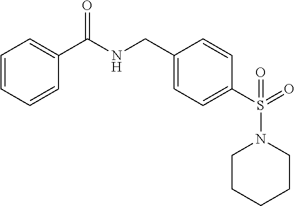 [SMILES: O=C(NCc1ccc(S(=O)(=O)N2CCCCC2)cc1)c1ccccc1]

### N-(4-(Piperidin-1-ylsulfonyl)benzyl)benzamide (MXC105)

¹H NMR (500 MHz, CDCl₃) δ 7.83 (d, 2H, J=8.3 Hz, 2H), 7.68 (d, J=8.3 Hz, 2H), 7.53 (m, 1H), 7.46 (m, 4H), 4.73 (d, J=6.1 Hz, 2H), 2.96 (m, 4H), 1.63 (m, 4H), 1.41 (m, 2H).

¹³C NMR (126 MHz, CDCl₃) δ 167.8, 144.0, 134.6, 133.9, 131.8, 128.6, 127.83, 127.76, 127.2, 46.9, 43.1, 25.1, 23.4.

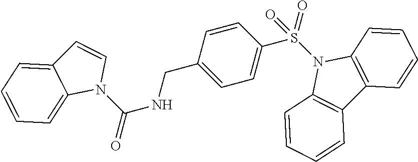 [SMILES: O=C(NCc1ccc(S(=O)(=O)n2c3ccccc3c3ccccc32)cc1)n1ccc2ccccc21]

### N-(4-((9H-Carbazol-9-yl)sulfonyl)benzyl)-1H-indole-1-carboxamide (MXC106)

¹H NMR (500 MHz, CDCl₃) δ 8.65 (t, J=5.7 Hz, 1H), 8.24 (d, J=8.4 Hz, 2H), 8.14 (d, J=8.3 Hz, 1H), 8.11 (d, J=7.7 Hz, 2H), 7.83 (d, J=8.6 Hz, 2H), 7.78 (d, J=3.7 Hz, 1H), 7.55 (m, 3H), 7.42 (m, 4H), 7.19 (m, 1H), 7.13 (app. t, J=7.4 Hz, 1H), 6.64 (d, J=3.2 Hz, 1H), 4.43 (d, J=5.7 Hz, 2H).

¹³C NMR (126 MHz, CDCl₃) δ 152.4, 147.2, 137.9, 135.8, 135.5, 129.9, 128.6, 128.4, 126.9, 126.2, 125.2, 124.9, 124.1, 122.5, 121.3, 121.2, 115.5, 115.1, 106.9, 43.3.

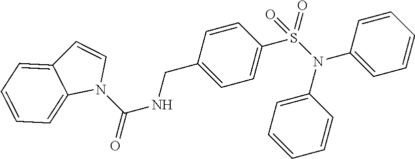 [SMILES: O=C(NCc1ccc(S(=O)(=O)N(c2ccccc2)c2ccccc2)cc1)n1ccc2ccccc21]

### N-(4-(N,N-Diphenylsulfamoyl)benzyl)-1H-indole-1-carboxamide (MXC107)

¹H NMR (500 MHz, CDCl₃) δ 8.83 (t, J=5.8 Hz, 1H), 8.24 (d, J=8.2 Hz, 1H), 7.90 (d, J=3.7 Hz, 1H), 7.66 (d, J=8.5 Hz, 2H), 7.59 (m, 3H), 7.35 (m, 4H), 7.26 (m, 7H), 7.17 (m, 1H), 6.70 (d, J=3.3 Hz, 1H), 4.60 (d, J=5.8 Hz, 2H).

¹³C NMR (126 MHz, CDCl₃) δ 152.5, 145.6, 141.6, 138.8, 135.9, 130.0, 128.7, 128.4, 128.1, 125.2, 124.2, 122.5, 121.2, 115.6, 107.0, 43.5 (two low-field carbon signals are missing).

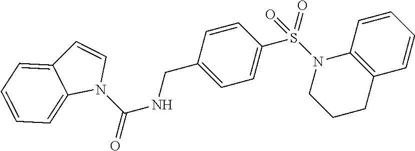 [SMILES: O=C(NCc1ccc(S(=O)(=O)N2CCCc3ccccc32)cc1)n1ccc2ccccc21]

### N-(4-((3,4-Dihydroquinolin-1(2H)-yl)sulfonyl)benzyl)-1H-indole-1-carboxamide (MXC108)

¹H NMR (500 MHz, CDCl₃) δ 8.78 (t, J=5.8 Hz, 1H), 8.20 (d, J=8.3 Hz, 1H), 7.87 (d, J=3.7 Hz, 1H), 7.58 (m, 4H), 7.50 (d, J=8.5 Hz, 2H), 7.23 (m, 1H), 7.15 (m, 2H), 7.03 (m, 2H), 6.68 (d, J=3.2 Hz, 1H), 4.53 (d, J=5.8 Hz, 2H), 3.75 (m, 2H), 2.42 (t, J=6.6 Hz, 2H), 1.59 (m, 2H).

¹³C NMR (126 MHz, CDCl₃) δ 152.5, 145.6, 138.1, 136.8, 135.8, 130.8, 129.9, 129.8, 128.3, 127.5, 126.7, 125.23, 125.17, 124.2, 123.9, 122.5, 121.2, 115.5, 106.9, 46.7, 43.5, 26.5, 21.7.

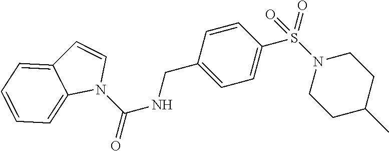 [SMILES: CC1CCN(S(=O)(=O)c2ccc(CNC(=O)n3ccc4ccccc43)cc2)CC1]

### N-(4-((4-Methylpiperidin-1-yl)sulfonyl)benzyl)-1H-indole-1-carboxamide (MXC109)

¹H NMR (500 MHz, CDCl₃) δ 8.83 (t, J=5.8 Hz, 1H), 8.22 (d, J=8.3 Hz, 1H), 7.90 (d, J=3.7 Hz, 1H), 7.70 (d, J=8.3 Hz, 2H), 7.59 (m, 3H), 7.23 (m, 1H), 7.16 (m, 1H), 6.70 (d, J=3.6 Hz, 1H), 4.58 (d, J=5.8 Hz, 2H), 3.57 (m, 2H), 2.15 (m, 2H), 1.60 (m, 2H), 1.25 (m, 1H), 1.09 (m, 2H), 0.81 (d, J=6.5 Hz, 3H).

¹³C NMR (126 MHz, CDCl₃) δ 152.5, 145.2, 135.9, 134.6, 129.9, 128.3, 128.1, 125.2, 124.2, 122.5, 121.2, 115.5, 107.0, 46.5, 43.5, 33.3, 29.8, 21.8.

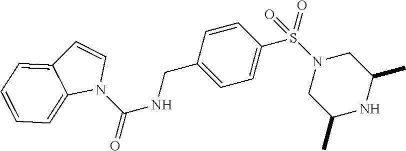 [SMILES: C[C@@H]1CN(S(=O)(=O)c2ccc(CNC(=O)n3ccc4ccccc43)cc2)C[C@H](C)N1]

### N-(4-(((3S,5R)-3,5-Dimethylpiperazin-1-yl)sulfonyl)benzyl)-1H-indole-1-carboxamide (MXC110)

¹H NMR (500 MHz, CDCl₃) δ 8.83 (t, J=5.8 Hz, 1H), 8.23 (d, J=8.2 Hz, 1H), 7.90 (d, J=3.7 Hz, 1H), 7.70 (d, J=8.4 Hz, 2H), 7.62 (d, J=8.4 Hz, 2H), 7.59 (d, J=7.7 Hz, 1H), 7.24 (m, 1H), 7.16 (m, 1H), 6.70 (d, J=3.6 Hz, 1H), 4.59 (d, J=5.7 Hz, 2H), 3.43 (dd, J=10.8, 2.0 Hz, 2H), 3.31 (br. s, 1H), 2.70 (m, 2H), 1.61 (app. t, J=10.7 Hz, 2H), 0.88 (d, J=6.3 Hz, 6H).

¹³C NMR (126 MHz, CDCl₃) δ 152.5, 145.3, 135.9, 134.0, 129.9, 128.3, 128.2, 125.2, 124.2, 122.5, 121.2, 115.5, 107.0, 52.3, 50.3, 43.5, 19.4.

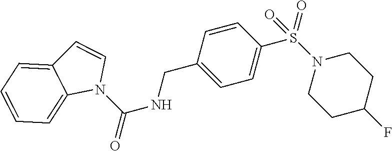 [SMILES: O=C(NCc1ccc(S(=O)(=O)N2CCC(F)CC2)cc1)n1ccc2ccccc21]

### N-(4-((4-Fluoropiperidin-1-yl)sulfonyl)benzyl)-1H-indole-1-carboxamide (MXC111)

¹H NMR (500 MHz, CDCl₃) δ 8.83 (t, J=5.8 Hz, 1H), 8.23 (d, J=8.3 Hz, 1H), 7.90 (d, J=3.7 Hz, 1H), 7.73 (d, J=8.4 Hz, 2H), 7.62 (d, J=8.4 Hz, 2H), 7.58 (d, J=7.7 Hz, 1H), 7.24 (m, 1H), 7.16 (m, 1H), 6.70 (d, J=3.7 Hz, 1H), 4.71 (m, 1H), 4.59 (d, J=5.8 Hz, 2H), 3.02 (m, 2H), 2.85 (m, 2H), 1.87 (m, 2H), 1.75 (m, 2H).

¹³C NMR (126 MHz, CDCl₃) δ 152.5, 145.5, 135.9, 134.3, 129.9, 128.4, 128.2, 125.2, 124.2, 122.5, 121.2, 115.5, 107.0, 87.2 (d, J=169.0 Hz), 43.5, 42.6 (d, J=6.2 Hz), 30.5 (d, J=19.9 Hz).

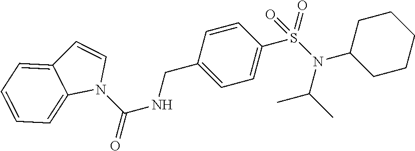 [SMILES: CC(C)N(C1CCCCC1)S(=O)(=O)c1ccc(CNC(=O)n2ccc3ccccc32)cc1]

### N-(4-(N-Cyclohexyl-N-isopropylsulfamoyl)benzyl)-1H-indole-1-carboxamide (MXC112)

¹H NMR (500 MHz, CDCl₃) δ 8.80 (t, J=5.8 Hz, 1H), 8.22 (dd, J=8.3, 0.7 Hz, 1H), 7.89 (d, J=3.7 Hz, 1H), 7.79 (d, J=8.4 Hz, 2H), 7.58 (d, J=7.5 Hz, 1H), 7.53 (d, J=8.5 Hz, 2H), 7.23 (m, 1H), 7.16 (m, 1H), 6.69 (d, J=3.7 Hz, 1H), 4.56 (d, J=5.8 Hz, 2H), 3.71 (m, 1H), 3.21 (m, 1H), 1.71 (m, 4H), 1.50 (m, 3H), 1.23 (m, 2H), 1.12 (d, J=6.8 Hz, 6H), 1.02 (m, 1H).

¹³C NMR (126 MHz, CDCl₃) δ 152.5, 144.2, 141.6, 135.9, 129.9, 128.0, 127.4, 125.2, 124.2, 122.5, 121.2, 115.5, 106.9, 57.0, 49.1, 43.5, 32.2, 26.3, 25.3, 22.1.

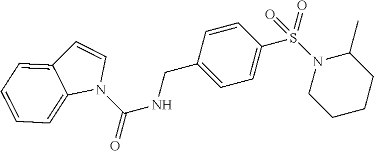 [SMILES: CC1CCCCN1S(=O)(=O)c1ccc(CNC(=O)n2ccc3ccccc32)cc1]

### N-(4-((2-Methylpiperidin-1-yl)sulfonyl)benzyl)-1H-indole-1-carboxamide (MXC113)

¹H NMR (500 MHz, CDCl₃) δ 8.81 (t, J=5.8 Hz, 1H), 8.22 (d, J=8.2 Hz, 1H), 7.89 (d, J=3.7 Hz, 1H), 7.76 (d, J=8.4 Hz, 2H), 7.58 (d, J=7.8 Hz, 1H), 7.56 (d, J=8.4 Hz, 2H), 7.24 (m, 1H), 7.16 (m, 1H), 6.70 (d, J=3.6 Hz, 1H), 4.57 (d, J=5.8 Hz, 2H), 4.08 (dd, J=6.7, 3.2 Hz, 1H), 3.56 (m, 1H), 2.92 (m, 1H), 1.43 (m, 5H), 1.16 (m, 1H), 0.96 (d, J=6.9 Hz, 3H).

¹³C NMR (126 MHz, CDCl₃) δ 152.5, 144.7, 139.9, 135.9, 129.9, 128.3, 127.4, 125.2, 124.2, 122.5, 121.2, 115.5, 106.9, 48.6, 43.5, 40.4, 30.3, 25.2, 18.1, 15.6.

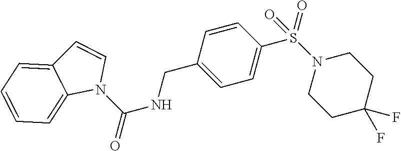 [SMILES: O=C(NCc1ccc(S(=O)(=O)N2CCC(F)(F)CC2)cc1)n1ccc2ccccc21]

### N-(4-((4,4-Difluoropiperidin-1-yl)sulfonyl)benzyl)-1H-indole-1-carboxamide (MXC114)

¹H NMR (500 MHz, CDCl₃) δ 8.83 (t, J=5.8 Hz, 1H), 8.23 (d, J=8.2 Hz, 1H), 7.90 (d, J=3.7 Hz, 1H), 7.75 (d, J=8.4 Hz, 2H), 7.63 (d, J=8.4 Hz, 2H), 7.58 (d, J=7.7 Hz, 1H), 7.24 (m, 1H), 7.16 (m, 1H), 6.70 (d, J=3.3 Hz, 1H), 4.60 (d, J=5.7 Hz, 2H), 3.04 (m, 4H), 2.04 (m, 4H).

¹³C NMR (126 MHz, CDCl₃) δ 152.5, 145.7, 135.9, 134.4, 129.9, 128.5, 128.1, 125.2, 124.2, 122.5, 122.2 (t, J=241.2 Hz), 121.2, 115.5, 107.0, 43.8 (t, J=5.6 Hz), 43.5, 33.2 (t, J=23.6 Hz).

NMR data for MHD001-MHD023:

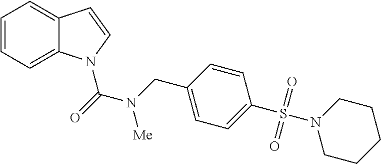 [SMILES: CN(Cc1ccc(S(=O)(=O)N2CCCCC2)cc1)C(=O)n1ccc2ccccc21]

### N-methyl-N-(4-(piperidin-1-ylsulfonyl)benzyl)-1H-indole-1-carboxamide (MHD001)

¹H NMR (500 MHz, CDCl₃) δ (ppm) 7.80-7.74 (m, 2H) 7.70 (d, J=8.3 Hz, 1H), 7.61 (d, J=7.8 Hz, 1H), 7.52-7.46 (m, 2H), 7.35-7.16 (m, 3H), 6.62 (d, J=3.5 Hz, 1H), 4.74 (s, 2H), 3.03 (s, 3H), 3.02-2.94 (m, 4H), 1.70-1.56 (m, 4H), 1.50-1.36 (m, 2H).

¹³C NMR (125 MHz, CDCl₃) δ (ppm) 155.3, 141.4, 136.1, 135.6, 129.5, 128.33, 128.31, 125.9, 123.9, 122.1, 121.2, 113.5, 106.4, 53.5, 46.9, 36.9, 25.2, 23.5.

LRMS (ESI) m/z: [M+H]⁺ Calcd for C₂₂H₂₆N₃O₃S: 412.2; Found 412.2.

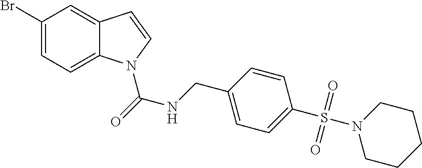 [SMILES: O=C(NCc1ccc(S(=O)(=O)N2CCCCC2)cc1)n1ccc2cc(Br)ccc21]

### 5-bromo-N-(4-(piperidin-1-ylsulfonyl)benzyl)-1H-indole-1-carboxamide (MHD002)

¹H NMR (500 MHz, CDCl₃-MeOH-d4 9:1) δ (ppm) 8.10 (d, J=8.9 Hz, 1H), 7.75-7.58 (m, 3H), 7.57-7.45 (m, 3H), 7.35 (d, J=8.9 Hz, 1H), 6.52 (d, J=3.1 Hz, 1H), 4.61 (s, 2H), 3.07-2.85 (m, 4H), 1.66-1.50 (m, 4H), 1.43-1.30 (m, 2H).

¹³C NMR (125 MHz, CDCl₃-MeOH-d4 9:1) δ (ppm) 152.4, 143.6, 135.1, 134.5, 131.6, 127.96, 127.92, 127.1, 124.7, 123.4, 116.5, 115.6, 106.6, 46.9, 43.8, 25.1, 23.4.

LRMS (ESI) m/z: [M+H]⁺ Calcd for C₂₁H₂₃BrN₃O₃S: 476.1; Found 476.1.

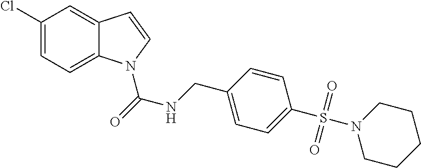 [SMILES: O=C(NCc1ccc(S(=O)(=O)N2CCCCC2)cc1)n1ccc2cc(Cl)ccc21]

### 5-chloro-N-(4-(piperidin-1-ylsulfonyl)benzyl)-1H-indole-1-carboxamide (MHD003)

¹H NMR (500 MHz, CDCl₃) δ (ppm) 8.17 (d, J=8.9 Hz, 1H), 7.65-7.46 (m, 4H), 7.38-7.27 (m, 3H), 6.64 (t, J=5.7 Hz, 1H), 6.58 (d, J=3.3 Hz, 1H), 4.69 (d, J=5.7 Hz, 2H), 3.04-2.82 (m, 4H), 1.72-1.54 (m, 4H), 1.50-1.32 (m, 2H).

¹³C NMR (125 MHz, CDCl₃) δ (ppm) 152.1, 143.4, 134.8, 134.1, 131.1, 128.1, 127.79, 127.72, 124.8, 124.6, 120.5, 116.0, 107.0, 46.9, 43.98, 25.1, 23.4

LRMS (ESI) m/z: [M+H]⁺ Calcd for C₂₁H₂₃ClN₃O₃S: 432.1; Found 432.2.

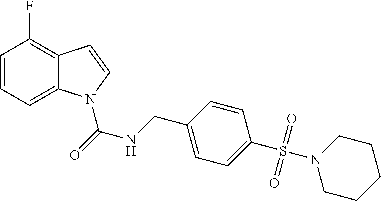 [SMILES: O=C(NCc1ccc(S(=O)(=O)N2CCCCC2)cc1)n1ccc2c(F)cccc21]

### 4-fluoro-N-(4-(piperidin-1-ylsulfonyl)benzyl)-1H-indole-1-carboxamide (MHD004)

¹H NMR (500 MHz, CDCl₃) δ (ppm) 7.97 (d, J=8.4 Hz, 1H), 7.62-7.54 (m, 2H), 7.50 (d, J=3.7 Hz, 1H), 7.48-7.40 (m, 2H), 7.29-6.80 (m, 2H), 6.76 (d, J=3.6 Hz, 1H), 6.45 (t, J=5.8 Hz, 1H), 4.72 (d, J=5.8 Hz, 2H), 3.06-2.80 (m, 4H), 1.78-1.51 (m, 4H), 1.48-1.32 (m, 2H).

¹³C NMR (125 MHz, CDCl₃; mixture of rotamers) δ (ppm) 156.0 (d, J=237.5 Hz), 152.1, 143.1, 137.6 (d, J=9.7 Hz), 135.1, 129.7, 127.94, 127.83, 125.1 (d, J=7.5 Hz), 123.7, 120.7, 119.1 (d, J=24.7 Hz), 115.3, 110.6 (d, J=5.0 Hz), 107.7 (d, J=18.4 Hz), 103.2, 46.9, 44.1, 25.1, 23.4.

LRMS (ESI) m/z: [M+H]⁺ Calcd for C₂₁H₂₃FN₃O₃S: 416.1; Found 416.2.

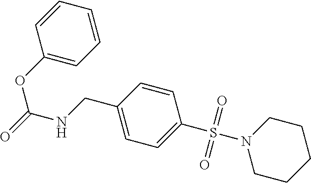 [SMILES: O=C(NCc1ccc(S(=O)(=O)N2CCCCC2)cc1)Oc1ccccc1]

### phenyl (4-(piperidin-1-ylsulfonyl)benzyl)carbamate (MHD005)

¹H NMR (500 MHz, CDCl₃) δ (ppm) 7.80-7.67 (m, 2H), 7.54-7.43 (m, 2H), 7.40-7.31 (m, 2H), 7.24-7.10 (m, 3H), 5.56 (br s, 1H), 4.53 (d, J=6.2 Hz, 2H), 3.13-2.82 (m, 4H), 1.76-1.54 (m, 4H), 1.52-1.36 (m, 2H).

¹³C NMR (125 MHz, CDCl₃) δ (ppm) 154.9, 150.9, 143.0, 135.3, 129.4, 128.1, 127.8, 125.6, 121.5, 46.9, 44.7, 25.2, 23.4.

LRMS (ESI) m/z: [M+H]⁺ Calcd for C₁₉H₂₃FN₂O₄S: 375.1; Found 375.2.

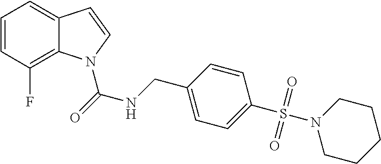 [SMILES: O=C(NCc1ccc(S(=O)(=O)N2CCCCC2)cc1)n1ccc2cccc(F)c21]

### 7-fluoro-N-(4-(piperidin-1-ylsulfonyl)benzyl)-1H-indole-1-carboxamide (MHD006)

¹H NMR (500 MHz, CDCl₃) δ (ppm) 7.96 (d, J=3.6 Hz, 1H), 7.81-7.67 (m, 2H), 7.62-7.49 (m, 2H), 7.48-7.39 (m, 1H), 7.25-6.82 (m, 3H), 6.66 (d, J=2.8 Hz, 1H), 4.75 (d, J=5.6 Hz, 2H), 3.09-2.89 (m, 4H), 1.74-1.59 (m, 4H), 1.46-1.35 (m, 2H).

¹³C NMR (125 MHz, CDCl₃) δ (ppm) 151.2, 149.0 (d, J=237.5 Hz), 142.6, 137.9, 135.8 (d, J=12.5 Hz), 129.4, 128.0, 125.3, 123.3 (d, J=8.3 Hz), 121.5, 118.1 (d, J=3.1 Hz), 110.4 (d, J=23.5 Hz), 106.6, 46.9, 44.5, 25.3, 23.5.

LRMS (ESI) m/z: [M+H]⁺ Calcd for C₂₁H₂₃FN₃O₃S: 416.1: Found 416.2.

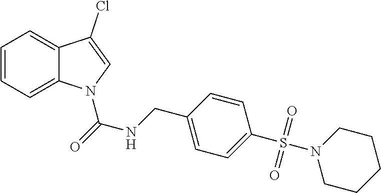 [SMILES: O=C(NCc1ccc(S(=O)(=O)N2CCCCC2)cc1)n1cc(Cl)c2ccccc21]

### 3-chloro-N-(4-(piperidin-1-ylsulfonyl)benzyl)-1H-indole-1-carboxamide (MHD007)

¹H NMR (500 MHz, CDCl₃) δ (ppm) 8.22 (d, J=8.4 Hz, 1H), 7.66-7.56 (m, 2H), 7.55-7.48 (m, 2H), 7.45-7.28 (m, 4H), 6.53 (t, J=5.8 Hz, 1H), 4.70 (d, J=5.8 Hz, 2H), 3.09-2.78 (m, 4H), 1.74-1.50 (m, 4H), 1.48-1.31 (m, 2H).

¹³C NMR (125 MHz, CDCl₃) δ (ppm) 151.7, 143.1, 135.0, 134.7, 127.96, 127.85, 127.6, 125.6, 123.0, 120.3, 118.7, 114.9, 112.2, 46.9, 44.1, 25.1, 23.4.

LRMS (ESI) m/z: [M+H]⁺ Calcd for C₂₁H₂₃ClN₃O₃S: 432.1; Found 432.2.

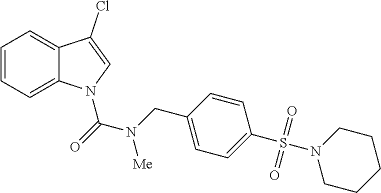 [SMILES: CN(Cc1ccc(S(=O)(=O)N2CCCCC2)cc1)C(=O)n1cc(Cl)c2ccccc21]

### 3-chloro-N-methyl-N-(4-(piperidin-1-ylsulfonyl)benzyl)-1H-indole-1-carboxamide (MHD008)

¹H NMR (500 MHz, CDCl₃) δ (ppm) 7.82-7.75 (m, 2H), 7.70 (d, J=8.3 Hz, 1H), 7.63 (d, J=7.8 Hz, 1H), 7.54-7.47 (m, 2H), 7.41-7.25 (m, 3H), 4.73 (s, 2H), 3.04 (s, 3H), 3.03-2.89 (m, 4H), 1.78-1.59 (m, 4H), 1.48-1.39 (m, 2H).

¹³C NMR (125 MHz, CDCl₃) δ (ppm) 151.7, 143.1, 135.0, 134.7, 127.96, 127.85, 127.6, 125.6, 123.0, 120.3, 118.7, 114.9, 112.2, 52.5, 46.9, 44.1, 25.1, 23.4.

LRMS (ESI) m/z: [M+H]⁺ Calcd for C₂₂H₂₅ClN₃O₃S: 446.1; Found 446.2.

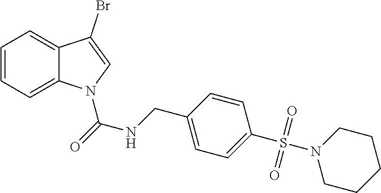 [SMILES: O=C(NCc1ccc(S(=O)(=O)N2CCCCC2)cc1)n1cc(Br)c2ccccc21]

### 3-bromo-N-(4-(piperidin-1-ylsulfonyl)benzyl)-1H-indole-1-carboxamide (MHD009)

¹H NMR (600 MHz, CDCl₃) δ (ppm) 8.22 (d, J=8.4 Hz, 1H), 7.65 (s, 1H), 7.62-7.57 (m, 3H), 7.51-7.45 (m, 2H), 7.44-7.32 (m, 2H), 6.45 (t, J=5.9 Hz, 1H), 4.74 (d, J=5.9 Hz, 2H), 3.11-2.87 (m, 4H), 1.75-1.62 (m, 4H), 1.51-1.36 (m, 2H).

¹³C NMR (150 MHz, CDCl₃) δ (ppm) 151.7, 143.1, 135.0, 134.7, 127.96, 127.85, 127.6, 125.6, 123.0, 120.3, 118.7, 114.9, 112.2, 46.9, 44.1, 25.1, 23.4.

LRMS (ESI) m/z: [M+H]⁺ Calcd for C₂₁H₂₃BrN₃O₃S: 476.1; Found 476.2.

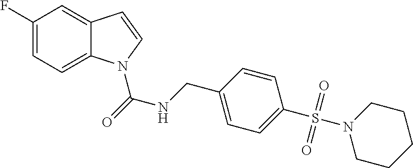 [SMILES: O=C(NCc1ccc(S(=O)(=O)N2CCCCC2)cc1)n1ccc2cc(F)ccc21]

### 5-fluoro-N-(4-(piperidin-1-ylsulfonyl)benzyl)-1H-indole-1-carboxamide (MHD010)

¹H NMR (600 MHz, CDCl₃) δ (ppm) 8.20 (dd, J=9.1, 4.6 Hz, 1H), 7.65-7.58 (m, 2H), 7.57 (d, J=3.7 Hz, 1H), 7.50-7.44 (m, 2H), 7.28-7.25 (m, 1H), 7.08 (td, J=9.1, 2.6 Hz, 1H), 6.64 (d, J=3.6 Hz, 1H), 6.40 (t, J=5.8 Hz, 1H), 4.74 (d, J=5.9 Hz, 2H), 3.17-2.83 (m, 4H), 1.74-1.61 (m, 4H), 1.50-1.36 (m, 2H).

¹³C NMR (150 MHz, CDCl₃; mixture of rotamers) δ (ppm) 159.0 (d, J=197.8 Hz), 152.1, 143.2, 135.2, 132.1, 130.8 (d, J=8.3 Hz), 129.7, 127.9, 127.8, 124.9, 120.7, 115.8 (d, J=7.5 Hz), 115.3, 112.5, 112.3, 107.5 (d, J=9.6 Hz), 106.4, 46.9, 44.0, 25.1, 23.4.

LRMS (ESI) m/z: [M+H]⁺ Calcd for C₂₁H₂₃FN₃O₃S: 416.1; Found 416.2.

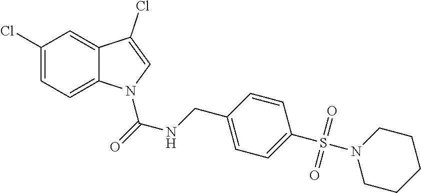 [SMILES: O=C(NCc1ccc(S(=O)(=O)N2CCCCC2)cc1)n1cc(Cl)c2cc(Cl)ccc21]

### 3,5-dichloro-N-(4-(piperidin-1-ylsulfonyl)benzyl)-1H-indole-1-carboxamide (MHD011)

¹H NMR (500 MHz, CDCl₃) δ (ppm) 8.18 (d, J=8.9 Hz, 1H), 7.63-7.56 (m, 3H), 7.54 (s, 1H), 7.49-7.41 (m, 2H), 7.35 (dd, J=8.9, 2.1 Hz, 1H), 6.34-6.19 (m, 1H), 4.70 (d, J=5.9 Hz, 2H), 3.04-2.92 (m, 4H), 1.68-1.59 (m, 4H), 1.49-1.30 (m, 2H).

¹³C NMR (125 MHz, CDCl₃) δ (ppm) 151.2, 142.7, 135.4, 133.2, 129.0, 128.7, 128.05, 128.01, 126.1, 121.1, 118.4, 116.3, 111.7, 46.9, 44.2, 25.1, 23.4.

LRMS (ESI) m/z: [M+H]⁺ Calcd for C₂₁H₂₂Cl₂N₃O₃S: 466.1; Found 466.1.

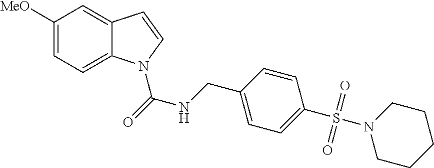 [SMILES: COc1ccc2c(ccn2C(=O)NCc2ccc(S(=O)(=O)N3CCCCC3)cc2)c1]

### 5-methoxy-N-(4-(piperidin-1-ylsulfonyl)benzyl)-1H-indole-1-carboxamide (MHD012)

¹H NMR (500 MHz, CDCl₃; mixture of rotamers) δ (ppm) 8.00 (d, J=9.0 Hz, 0.9H), 7.78-7.66 (m, 1.91H), 7.58-7.47 (m, 1.93H), 7.44 (d, J=3.7 Hz, 0.95H), 7.10-7.01 (m, 0.95H), 6.99-6.87 (m, 1.1H), 6.83 (d, J=8.5 Hz, 0.4H), 6.59 (d, J=3.6 Hz, 0.96H), 6.05-5.84 (m, 0.91H), 4.73 (d, J=5.9 Hz, 2H), 3.85 (s, 3H), 3.05-2.84 (m, 4H), 1.71-1.58 (m, 4H), 1.46-1.33 (m, 2H).

¹³C NMR (125 MHz, CDCl₃; mixture of rotamers) δ (ppm) 155.8, 152.1, 143.0, 135.7, 131.0, 130.1, 129.7, 128.2, 128.0, 124.2, 115.3, 115.0, 113.8, 107.6, 103.6, 55.7, 46.9, 44.2, 25.2, 23.5.

LRMS (ESI) m/z: [M+H]⁺ Calcd for C₂₂H₂₆N₃O₄S: 428.2; Found 428.2.

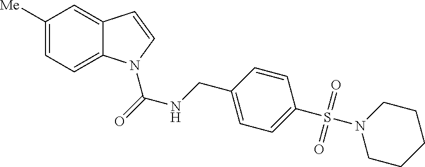 [SMILES: Cc1ccc2c(ccn2C(=O)NCc2ccc(S(=O)(=O)N3CCCCC3)cc2)c1]

### 5-methyl-N-(4-(piperidin-1-ylsulfonyl)benzyl)-1H-indole-1-carboxamide (MHD013)

¹H NMR (500 MHz, CDCl₃; mixture of rotamers) δ (ppm) 7.99 (d, J=8.5 Hz, 0.92H), 7.68-7.58 (m, 1.98H), 7.52-7.43 (m, 2.68H), 7.42-7.34 (m, 0.9H), 7.25-7.20 (m, 0.22H), 7.14 (dd, J=8.5, 1.6 Hz, 0.83H), 6.95-6.89 (m, 0.13H), 6.86-6.81 (m, 0.57H), 6.57 (d, J=3.7 Hz, 0.93H), 6.21 (t, J=5.9 Hz, 0.9H), 6.05-5.84 (m, 0.91H), 4.72 (d, J=5.9 Hz, 2H), 3.10-2.86 (m, 4H), 2.44 (s, 3H), 1.70-1.51 (m, 4H), 1.48-1.32 (m, 2H).

¹³C NMR (125 MHz, CDCl₃; mixture of rotamers) δ (ppm) 155.6, 152.3, 143.2, 135.3, 133.5, 132.0, 130.5, 129.7, 128.0, 127.9, 125.9, 123.9, 121.1, 120.8, 115.3, 113.9, 107.4, 46.9, 44.1, 25.1, 23.4, 21.3.

LRMS (ESI) m/z: [M+H]⁺ Calcd for C₂₂H₂₆N₃O₃S: 412.2; Found 412.2.

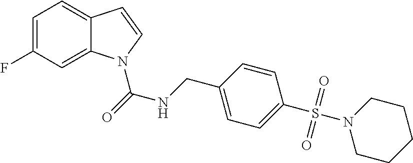 [SMILES: O=C(NCc1ccc(S(=O)(=O)N2CCCCC2)cc1)n1ccc2ccc(F)cc21]

### 6-fluoro-N-(4-(piperidin-1-ylsulfonyl)benzyl)-1H-indole-1-carboxamide (MHD014)

¹H NMR (500 MHz, CDCl₃; mixture of rotamers) δ (ppm) 7.99 (dd, J=10.4, 2.3 Hz, 0.94H), 7.63-7.54 (m, 1.94H), 7.53-7.40 (m, 3.85H), 7.25-7.14 (m, 0.14H), 7.10-6.80 (m, 1.28H), 6.63 (d, J=3.7 Hz, 0.93H), 6.54-6.49 (m, 0.08H), 6.31 (t, J=5.9 Hz, 0.86H), 4.72 (d, J=5.9 Hz, 2H), 3.03-2.82 (m, 4H), 1.70-1.58 (m, 4H), 1.47-1.35 (m, 2H).

¹³C NMR (125 MHz, CDCl₃; mixture of rotamers) δ (ppm) 161.0 (d, J=238.9 Hz), 152.1, 143.2, 135.8 (d, J=12.9 Hz), 135.2, 129.7, 128.0, 127.8, 126.2, 123.60, 123.57, 121.71, 121.63, 120.8, 115.3, 110.5 (d, J=24.2 Hz), 107.7, 102.2 (d, J=28.4 Hz), 46.9, 44.0, 25.1, 23.4.

LRMS (ESI) m/z: [M+H]⁺ Calcd for C₂₁H₂₃FN₃O₃S: 416.1; Found 416.2.

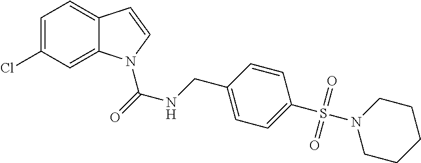 [SMILES: O=C(NCc1ccc(S(=O)(=O)N2CCCCC2)cc1)n1ccc2ccc(Cl)cc21]

### 6-chloro-N-(4-(piperidin-1-ylsulfonyl)benzyl)-1H-indole-1-carboxamide (MHD015)

¹H NMR (500 MHz, CDCl₃; mixture of rotamers) δ (ppm) 8.28 (brs, 0.92H), 7.67-7.55 (m, 2H), 7.54-7.41 (m, 3.4H), 7.25-7.15 (m, 1H), 6.97-6.87 (m, 0.24H), 6.86-6.79 (m, 0.44H), 6.63 (d, J=3.6 Hz, 1H), 6.30 (t, J=5.8 Hz, 1H), 4.72 (d, J=5.9 Hz, 2H), 3.05-2.85 (m, 4H), 1.70-1.59 (m, 4H), 1.46-1.33 (m, 2H).

¹³C NMR (125 MHz, CDCl₃; mixture of rotamers) δ (ppm) 155.6, 152.3, 143.2, 135.3, 133.5, 132.0, 130.5, 129.7, 128.0, 127.9, 125.9, 123.9, 121.1, 120.8, 115.3, 113.9, 107.4, 46.9, 44.1, 25.1, 23.4, 21.3.

LRMS (ESI) m/z: [M+H]⁺ Calcd for C₂₁H₂₃ClN₃O₃S: 432.1; Found 432.1.

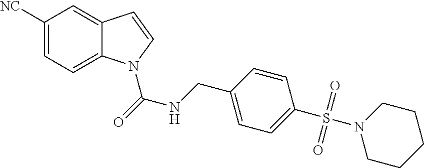 [SMILES: N#Cc1ccc2c(ccn2C(=O)NCc2ccc(S(=O)(=O)N3CCCCC3)cc2)c1]

### 5-cyano-N-(4-(piperidin-1-ylsulfonyl)benzyl)-1H-indole-1-carboxamide (MHD016)

¹H NMR (500 MHz, CDCl₃; mixture of rotamers) δ (ppm) 8.36 (d, J=8.7 Hz, 0.92H), 8.03-7.89 (m, 1H), 7.66-7.51 (m, 3.72H), 7.50-7.39 (m, 2.05H), 6.96-6.79 (m, 0.38H), 6.73 (d, J=3.5 Hz, 1H), 6.46 (brs, 1H), 4.74 (d, J=5.8 Hz, 2H), 3.02-2.82 (m, 4H), 1.68-1.58 (m, 4H), 1.48-1.34 (m, 2H).

¹³C NMR (125 MHz, CDCl₃; mixture of rotamers) δ (ppm) 151.6, 142.8, 137.5, 135.3, 129.8, 129.7, 128.0, 127.9, 127.4, 126.1, 125.6, 119.8, 115.9, 115.3, 107.6, 105.9, 46.9, 44.1, 25.1, 23.4.

LRMS (ESI) m/z: [M+H]⁺ Calcd for C₂₂H₂₃N₄O₃S: 423.1; Found 423.1.

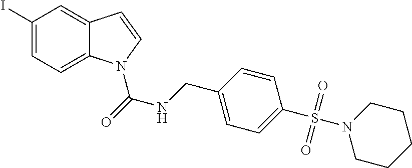 [SMILES: O=C(NCc1ccc(S(=O)(=O)N2CCCCC2)cc1)n1ccc2cc(I)ccc21]

### 5-iodo-N-(4-(piperidin-1-ylsulfonyl)benzyl)-1H-indole-1-carboxamide (MHD017)

¹H NMR (500 MHz, CDCl₃; mixture of rotamers) δ (ppm) 8.05-7.87 (m, 1.8H), 7.72-7.63 (m, 1.75H), 7.59 (d, J=8.7 Hz, 0.9H), 7.57-7.46 (m, 1.83H), 7.42 (d, J=3.6 Hz, 0.96H), 7.25-7.21 (m, 0.16H), 6.98-6.89 (m, 0.29H), 6.87-6.80 (m, 0.47H), 6.58 (d, J=3.6 Hz, 0.92H), 6.10 (brs, 1H), 4.73 (d, J=5.9 Hz, 2H), 3.04-2.89 (m, 4H), 1.69-1.59 (m, 4H), 1.47-1.38 (m, 2H).

¹³C NMR (125 MHz, CDCl₃; mixture of rotamers) δ (ppm) 151.8, 142.8, 135.6, 134.8, 132.9, 132.3, 130.0, 129.7, 128.1, 127.9, 124.1, 116.5, 115.3, 106.8, 86.5, 46.9, 44.2, 25.1, 23.4.

LRMS (ESI) m/z: [M+H]⁺ Calcd for C₂₁H₂₃IN₃O₃S: 524.0; Found 524.0.

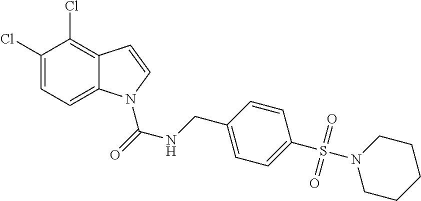 [SMILES: O=C(NCc1ccc(S(=O)(=O)N2CCCCC2)cc1)n1ccc2c(Cl)c(Cl)ccc21]

### 4,5-dichloro-N-(4-(piperidin-1-ylsulfonyl)benzyl)-1H-indole-1-carboxamide (MHD018)

¹H NMR (500 MHz, CDCl₃; mixture of rotamers) δ (ppm) 8.09 (dd, J=8.9, 0.7 Hz, 0.8H), 7.75-7.70 (m, 0.28H), 7.67-7.60 (m, 1.58H), 7.55-7.45 (m, 2.69H), 7.41-7.34 (m, 1.16H), 7.25-7.13 (m, 0.67H), 6.95-6.81 (m, 0.17H), 6.77 (dd, J=3.7, 0.7 Hz, 0.81H), 6.25 (t, J=5.8 Hz, 0.87H), 4.72 (d, J=5.9 Hz, 1.67H), 4.53 (d, J=6.2 Hz, 0.33H), 3.05-2.88 (m, 4H), 1.70-1.59 (m, 4H), 1.47-1.37 (m, 2H).

¹³C NMR (125 MHz, CDCl₃; mixture of rotamers) δ (ppm) 151.6, 142.7, 135.5, 134.2, 130.1, 129.4, 128.13, 128.08, 127.9, 127.8, 126.3, 126.0, 124.8, 124.2, 121.5, 114.3, 106.3, 46.9, 44.2, 25.16, 25.13, 23.4.

LRMS (ESI) m/z: [M+H]⁺ Calcd for C₂₁H₂₂Cl₂N₃O₃S: 466.1; Found 466.1.

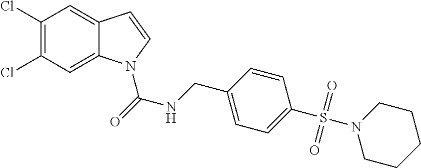 [SMILES: O=C(NCc1ccc(S(=O)(=O)N2CCCCC2)cc1)n1ccc2cc(Cl)c(Cl)cc21]

### 5,6-dichloro-N-(4-(piperidin-1-ylsulfonyl)benzyl)-1H-indole-1-carboxamide (MHD019)

¹H NMR (500 MHz, CDCl₃; mixture of rotamers) δ (ppm) 8.43 (s, 0.88H), 7.73-7.58 (m, 2.63H), 7.50-7.33 (m, 2.94H), 7.25-7.12 (m, 0.52H), 6.96-6.79 (m, 0.38H), 6.59 (dd, J=3.7, 0.7 Hz, 0.85H), 6.31-6.16 (m, 0.84H), 4.72 (d, J=5.9 Hz, 1.88H), 4.54 (d, J=6.3 Hz, 0.12H), 3.05-2.86 (m, 4H), 1.70-1.59 (m, 4H), 1.48-1.37 (m, 2H).

¹³C NMR (125 MHz, CDCl₃; mixture of rotamers) δ (ppm) 151.6, 142.8, 135.4, 134.4, 129.7, 129.4, 128.6, 128.1, 127.9, 126.7, 124.8, 121.9, 116.8, 115.3, 107.0, 46.9, 44.1, 25.16, 25.13, 23.4.

LRMS (ESI) m/z: [M+H]⁺ Calcd for C₂₁H₂₂Cl₂N₃O₃S: 466.1; Found 466.1.

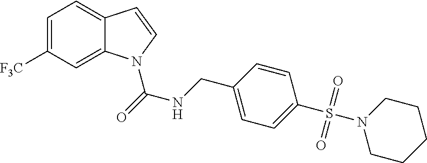 [SMILES: O=C(NCc1ccc(S(=O)(=O)N2CCCCC2)cc1)n1ccc2ccc(C(F)(F)F)cc21]

### N-(4-(piperidin-1-ylsulfonyl)benzyl)-6-(trifluoromethyl)-1H-indole-1-carboxamide (MHD020)

¹H NMR (500 MHz, CDCl₃; mixture of rotamers) δ (ppm) 8.57 (s, 0.84H), 7.69 (d, J=8.3 Hz, 0.95H), 7.67-7.62 (m, 1.74H), 7.59 (d, J=3.7 Hz, 0.9H), 7.54-7.36 (m, 2.93H), 7.25-7.12 (m, 0.34H), 6.96-6.79 (m, 0.5H), 6.72 (dd, J=3.7, 0.6 Hz, 0.9H), 6.34-6.18 (m, 0.94H), 4.80-4.53 (m, 2H), 3.10-2.85 (m, 4H), 1.70-1.59 (m, 4H), 1.47-1.37 (m, 2H).

¹³C NMR (125 MHz, CDCl₃; mixture of rotamers) δ (ppm) 151.8, 142.8, 135.5, 134.8, 132.3, 129.7, 128.1, 127.9, 125.7, 121.5, 119.3, 115.3, 112.56, 112.53, 107.6, 46.9, 44.2, 25.1, 23.4.

LRMS (ESI) m/z: [M+H]⁺ Calcd for C₂₂H₂₃F₃N₃O₃S: 466.1; Found 466.1.

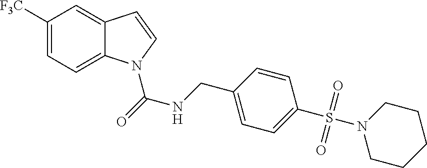 [SMILES: O=C(NCc1ccc(S(=O)(=O)N2CCCCC2)cc1)n1ccc2cc(C(F)(F)F)ccc21]

### N-(4-(piperidin-1-ylsulfonyl)benzyl)-5-(trifluoromethyl)-1H-indole-1-carboxamide (MHD021)

¹H NMR (500 MHz, CDCl₃; mixture of rotamers) δ (ppm) 8.33 (d, J=8.8 Hz, 0.89H), 7.89 (s, 0.91H), 7.76-7.61 (m, 2H), 7.60-7.54 (m, 1.74H), 7.52-7.33 (m, 2.22H), 7.25-7.11 (m, 0.28H), 6.95-6.79 (m, 0.14H), 6.74 (d, J=3.7 Hz, 0.9H), 6.28 (t, J=5.7 Hz, 0.93H), 4.74 (d, J=5.7 Hz, 1.8H), 4.66-4.52 (m, 0.2H), 3.09-2.86 (m, 4H), 1.72-1.58 (m, 4H), 1.49-1.35 (m, 2H).

¹³C NMR (125 MHz, CDCl₃; mixture of rotamers) δ (ppm) 151.8, 142.8, 137.1, 135.5, 129.6, 128.1, 127.9, 125.0, 121.25, 121.23, 118.65, 118.62, 115.1, 108.0, 46.9, 44.2, 25.1, 23.4.

LRMS (ESI) m/z: [M+H]⁺ Calcd for C₂₂H₂₃F₃N₃O₃S: 466.1; Found 466.1.

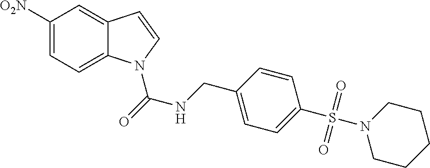 [SMILES: O=C(NCc1ccc(S(=O)(=O)N2CCCCC2)cc1)n1ccc2cc([N+](=O)[O-])ccc21]

### 5-nitro-N-(4-(piperidin-1-ylsulfonyl)benzyl)-1H-indole-1-carboxamide (MHD022)

¹H NMR (500 MHz, CDCl₃; mixture of rotamers) δ (ppm) 8.61 (s, 0.15H), 8.53 (d, J=2.1 Hz, 0.97H), 8.42-8.36 (m, 0.92H), 8.27-8.19 (m, 0.9H), 8.15-8.10 (m, 0.12H), 7.65 (d, J=3.7 Hz, 0.94H), 7.63-7.55 (m, 1.86H), 7.51-7.36 (m, 2.16H), 6.82 (d, J=3.6 Hz, 0.95H), 6.74 (s, 0.14H), 6.48 (t, J=5.5 Hz, 0.94H), 4.75 (d, J=5.9 Hz, 2H), 3.15-2.84 (m, 4H), 1.73-1.59 (m, 4H), 1.47-1.36 (m, 2H).

¹³C NMR (125 MHz, CDCl₃; mixture of rotamers) δ (ppm) 151.5, 143.6, 142.7, 138.7, 135.4, 129.6, 128.0, 127.9, 126.3, 119.7, 117.5, 115.3, 108.6, 46.9, 44.2, 25.1, 23.4.

LRMS (ESI) m/z: [M+H]⁺ Calcd for C₂₁H₂₃N₄O₅S: 443.1; Found 443.1.

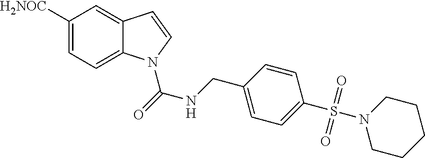 [SMILES: NC(=O)c1ccc2c(ccn2C(=O)NCc2ccc(S(=O)(=O)N3CCCCC3)cc2)c1]

### N1-(4-(piperidin-1-ylsulfonyl)benzyl)-1H-indole-1,5-dicarboxamide (MHD023)

¹H NMR (500 MHz, 9:1 CDCl₃-MeOH-d₄; mixture of rotamers) δ (ppm) 8.22 (d, J=8.8 Hz, 1H), 8.11-7.98 (m, 1.73H), 7.71-7.52 (m, 3.95H), 7.51-7.32 (m, 2.13H), 7.22-7.16 (m, 0.12H), 6.60 (d, J=3.6 Hz, 0.98H), 6.53-6.48 n(m, 0.11H), 4.66-4.50 (m, 2H), 2.97-2.81 (m, 4H), 1.64-1.49 (m, 4H), 1.41-1.28 (m, 2H).

¹³C NMR (125 MHz, 9:1 CDCl₃-MeOH-d₄; mixture of rotamers) δ (ppm) 175.0, 156.6, 147.7, 141.7, 138.9, 133.7, 131.9, 131.8, 131.1, 129.1, 127.1, 125.0, 124.98, 118.8, 111.7, 50.8, 47.9, 47.7, 29.0, 27.3.

LRMS (ESI) m/z: [M+H]⁺ Calcd for C₂₂H₂₅N₄O₄S: 441.2; Found 441.2.

### Testing of MCX Analogs

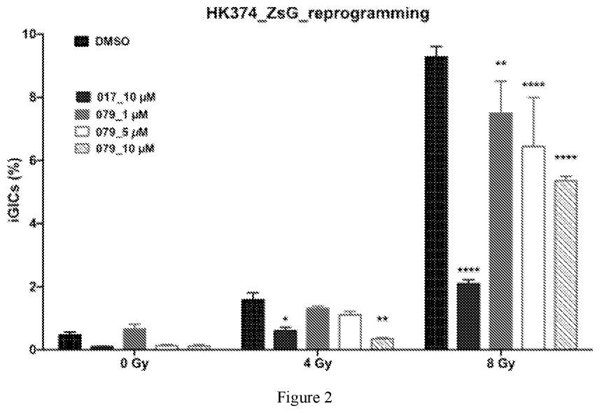

The newly synthesized analogs were tested for their ability to reduce self-renewal of patient-derived glioblastoma-initiating cells alone or in combination with a single radiation dose of 4 Gy. This led to the identification of the lead compounds MCX017, its analog MCX079, and MCX021 with in silico prediction of BBB penetration for MCX017 and MCX079. Furthermore, MCX017 and MCX079 prevented radiation-induced phenotype conversion of non-stem glioblastoma cells into GICs (FIG. 2).

### Target Identification

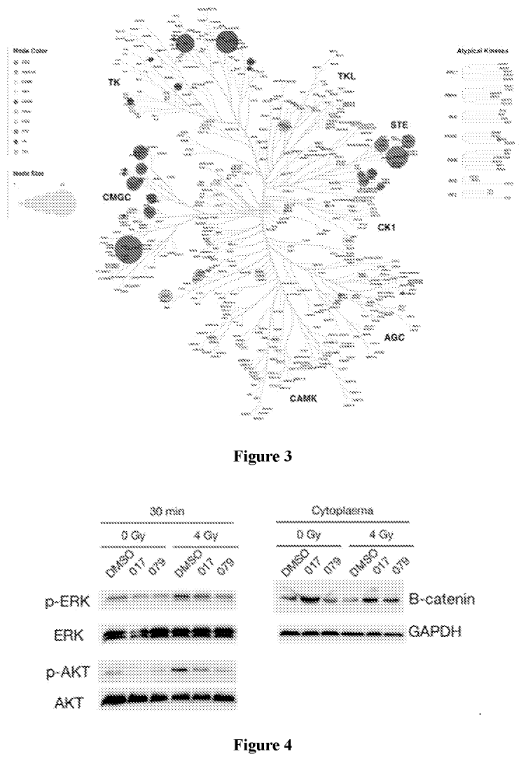

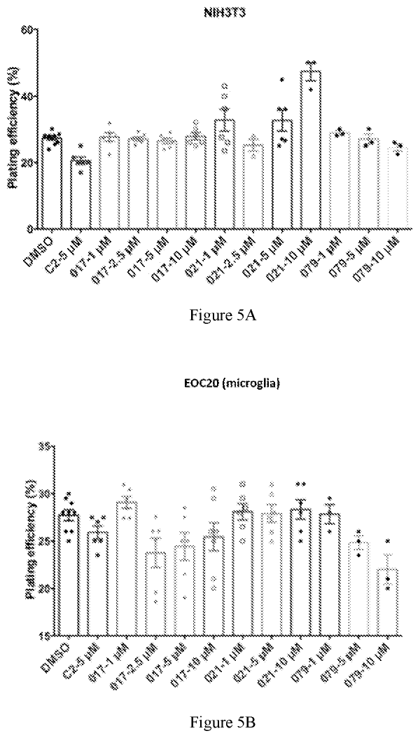

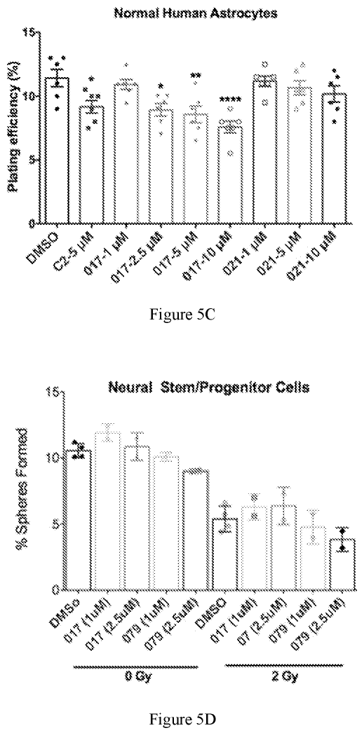

Compound 2 was identified in an unbiased phenotypic screen and MCX analogs were synthesized without knowledge of the actual molecular target. To identify the target of MCX017 a whole kinome screen was performed, which identified MCX017 as a multikinase inhibitor with activity against FLT3, TNIK and potentially JNK3 (FIG. 3). Western blotting confirmed inhibition of the downstream targets with downregulation of p-Erk, p-Akt and upregulation of cytosolic beta-catenin (FIG. 4). Testing of MCX017, MCX079 and MCX21 in normal tissue cell lines showed no relevant toxicity alone or in combination with radiation (FIGS. 5A-D).

### Material & Methods

### Cell Culture

Primary human glioma cell lines were established at UCLA as described (Hemmati et al., PNAS 2003; Characteristics of specific gliomasphere lines can be found in Laks et al., Neuro-Oncology 2016). Primary glioblastoma cells were propagated as gliomaspheres in serum-free conditions in T25 flasks in DMEM/F12, supplemented with B27, EGF, bFGF and heparin as described previously (Vlashi et al., Journal of the NCI, 2009, 101, 350-9). All cells were grown in a humidified atmosphere at 37° C. with 5% CO₂. The unique identity of all patient-derived specimen was confirmed by DNA fingerprinting (Laragen, Culver City, CA). All lines were routinely tested for mycoplasma infection (MycoAlert, Lonza).

### Irradiation

Cells were irradiated at room temperature using an experimental X-ray irradiator (Gulmay Medical Inc. Atlanta, GA) at a dose rate of 5.519 Gy/min for the time required to apply a prescribed dose. The X-ray beam was operated at 300 kV and hardened using a 4 mm Be, a 3 mm Al, and a 1.5 mm Cu filter and calibrated using NIST-traceable dosimetry. Corresponding controls were sham irradiated.

### Reprogramming Assay

Primary glioblastoma cells were engineered to express a fusion protein of ZsGreen and the C-terminal degron of ornithine decarboxylase. This construct reports for lack of 26S proteasome activity and cells with low proteasome activity are enriched for cells with tumor-initiating properties. For reprogramming experiments, cells with low proteasome activity (ZsGreen-positive) were removed by FACS. ZsGreen-negative cells were treated and 5 days later the number of radiation-induced ZsGreen-positive cells was assessed by flow cytometry. Results are shown in FIG. 27.

### Sphere-Forming Assay (SFA)

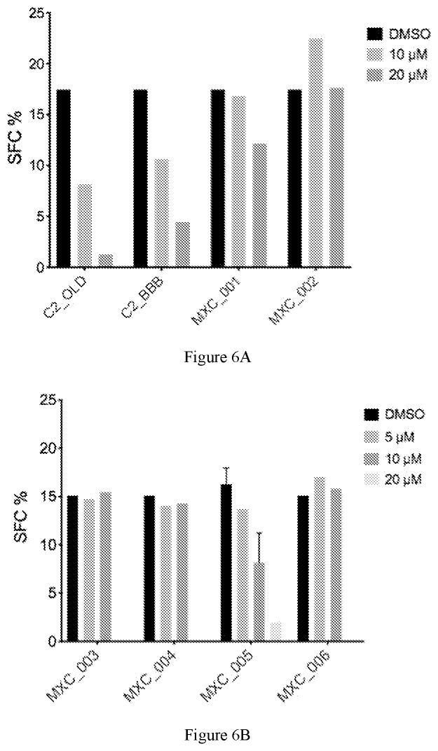

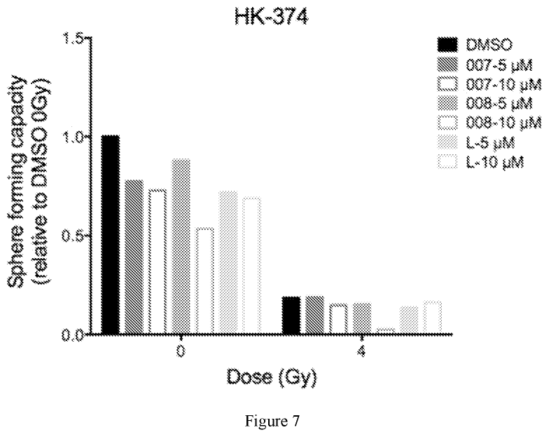

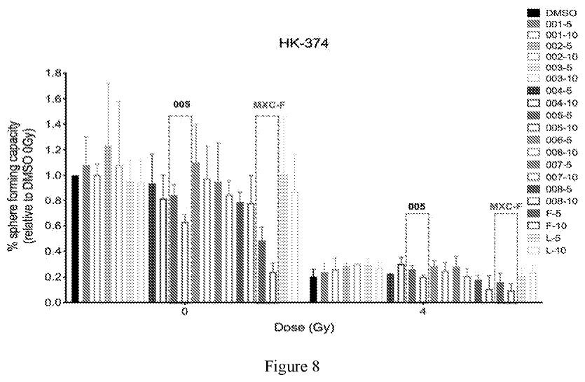

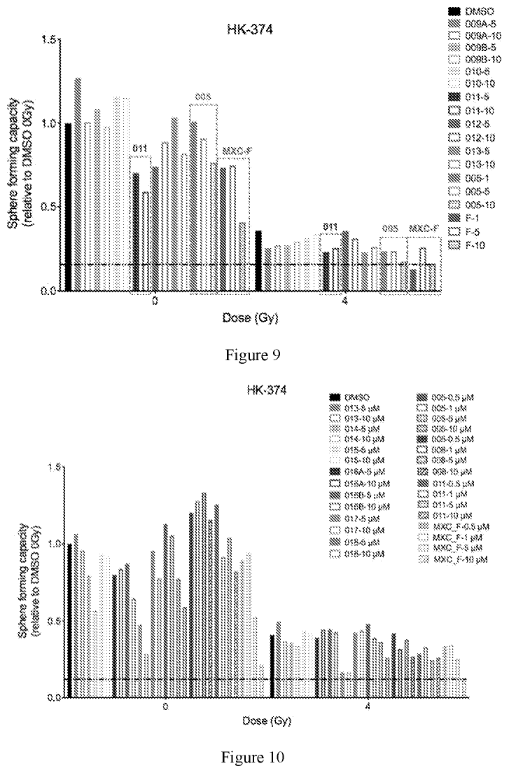

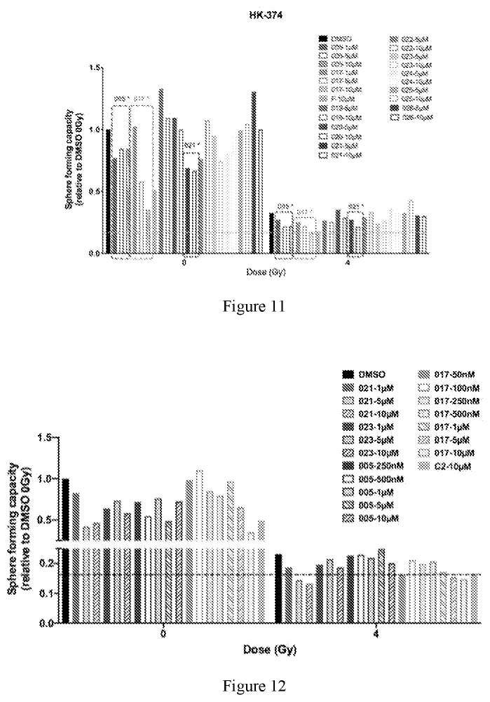

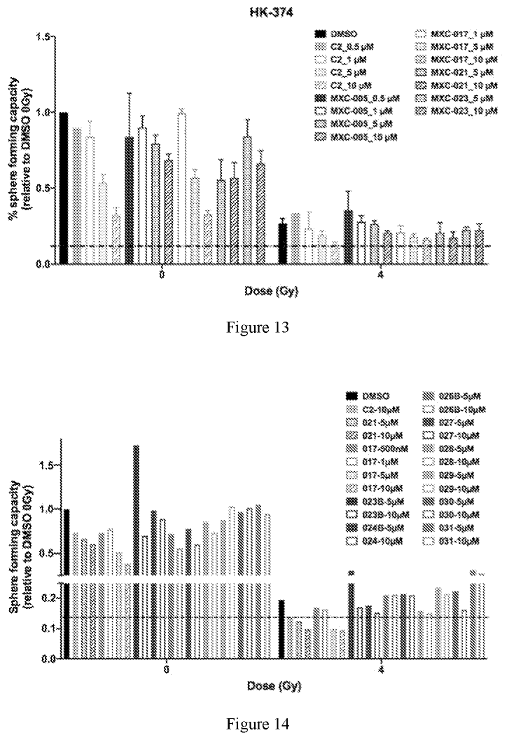

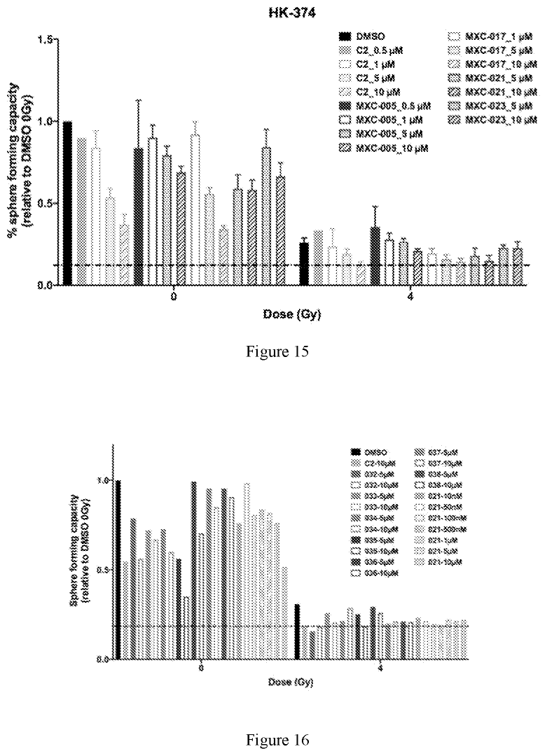

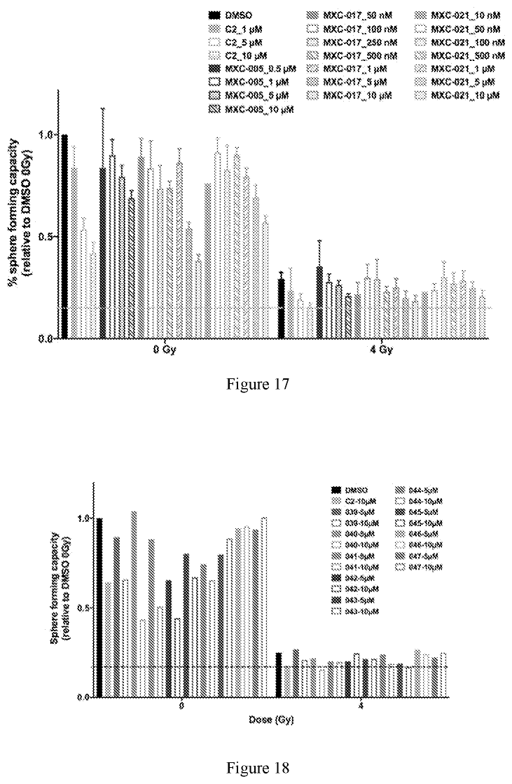

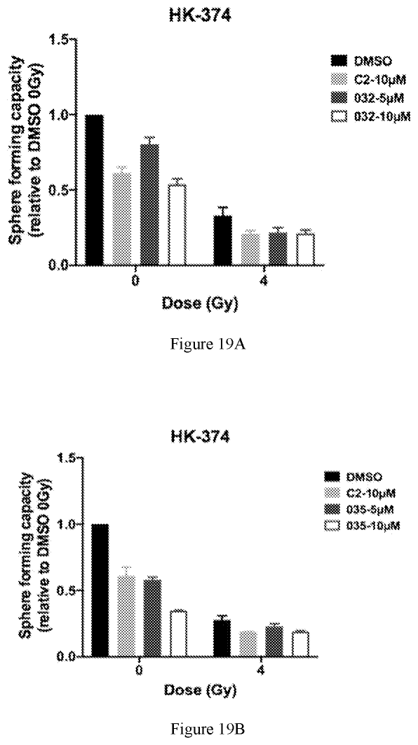

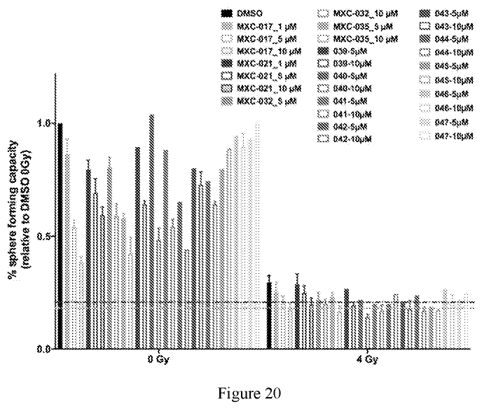

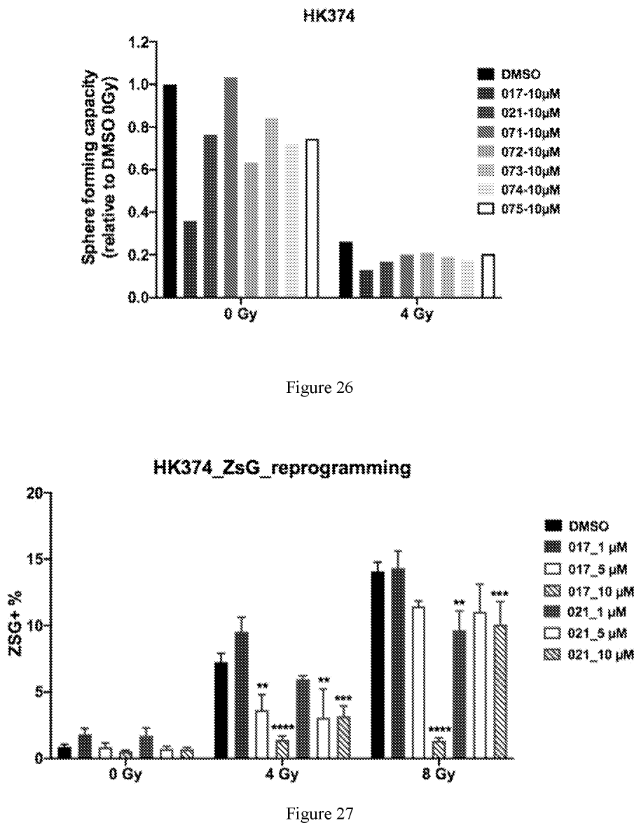

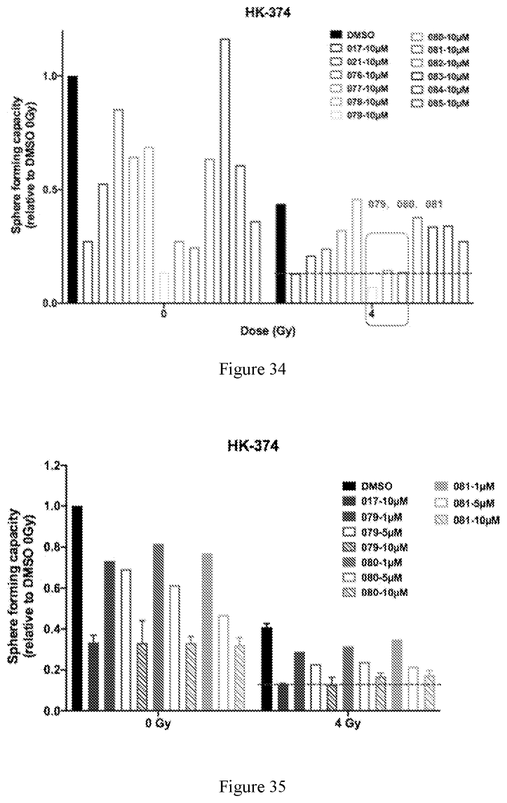

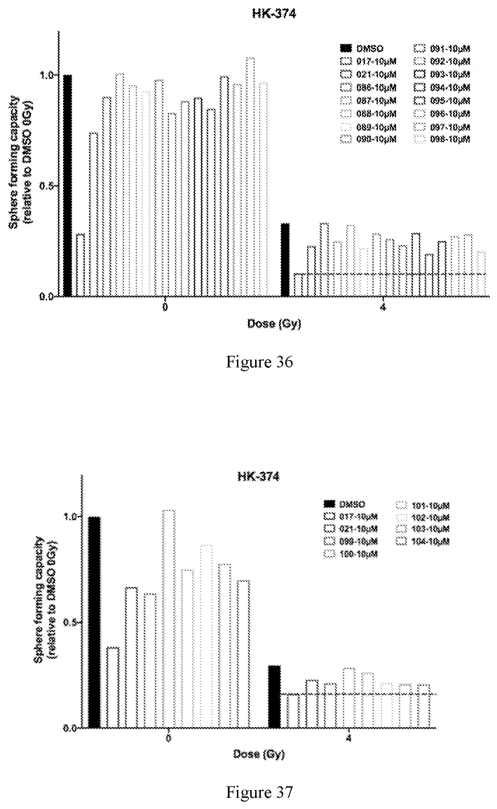

Cells were seeded under serum-free conditions into untreated plates in DMEM/F12 media, supplemented with 10 ml/500 mL of B27 (Invitrogen), 0.145 U/ml recombinant insulin (Eli Lilly, Indiana), 0.68 U/mL heparin (Fresenius Kabi, Illinois), 20 ng/ml fibroblast growth factor 2 (bFGF, Sigma) and 20 ng/ml epidermal growth factor (EGF, Sigma). After 10 days in culture, spheres formed from singles cells were counted. Certain compounds tested in sphere-forming assay according to the present disclosure are shown below. Results are shown in FIGS. 6, 7, 8, 9-19, 26, 33-37.

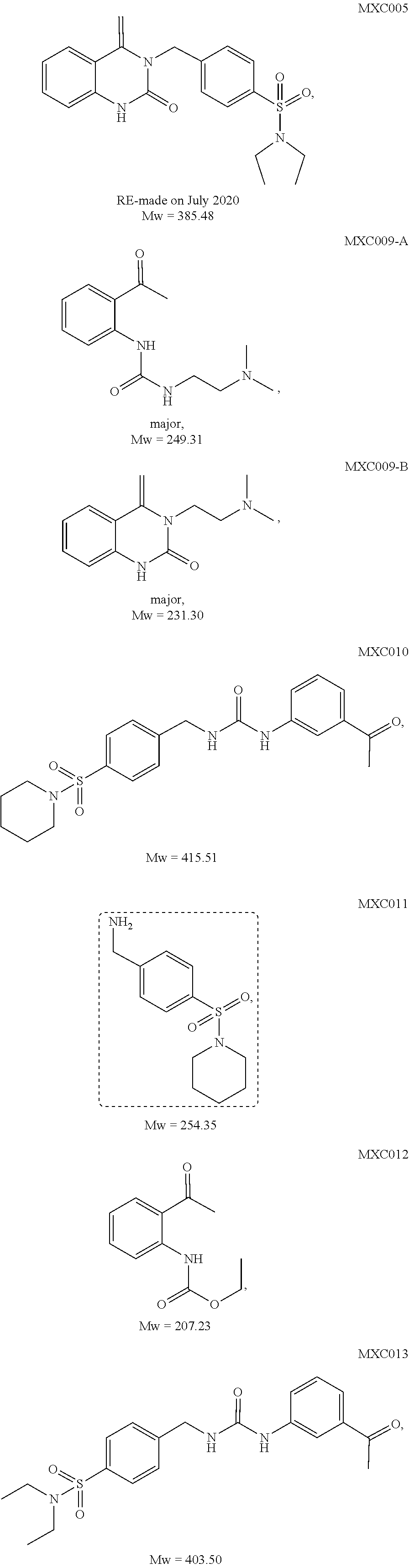

intermediate product of MXC-F
Compounds according to the present disclosure.

### Plating Efficiency Assay

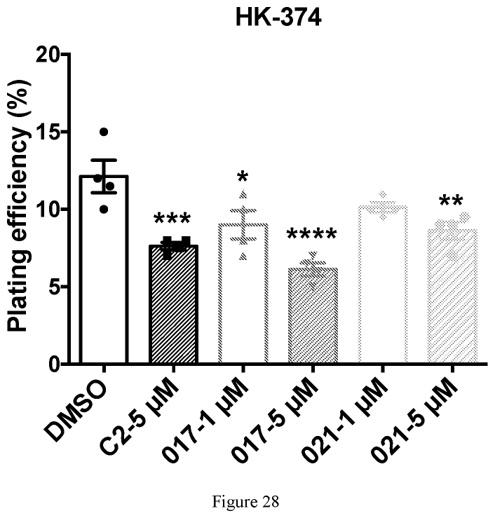

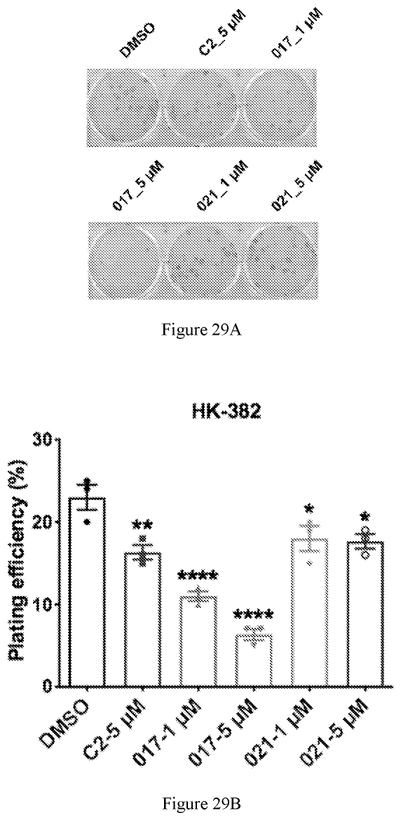

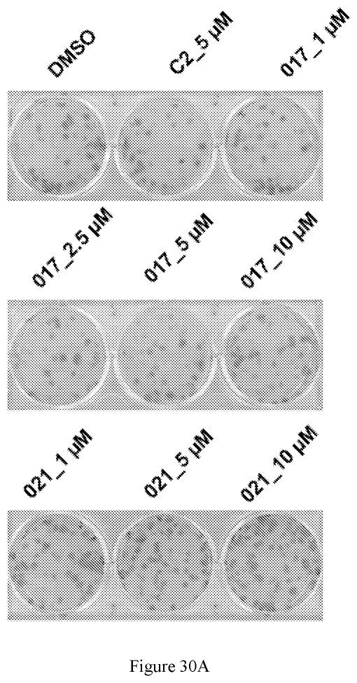

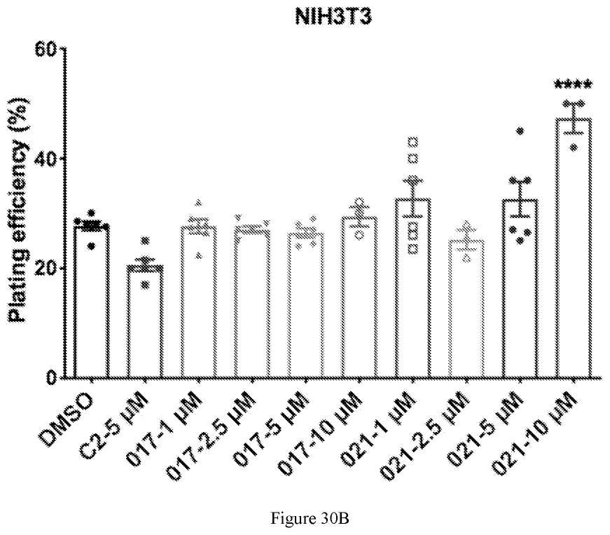

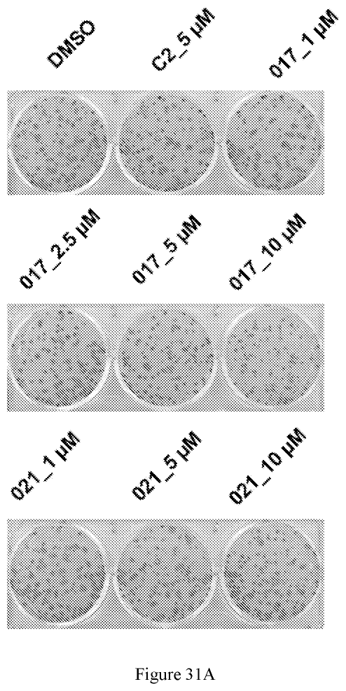

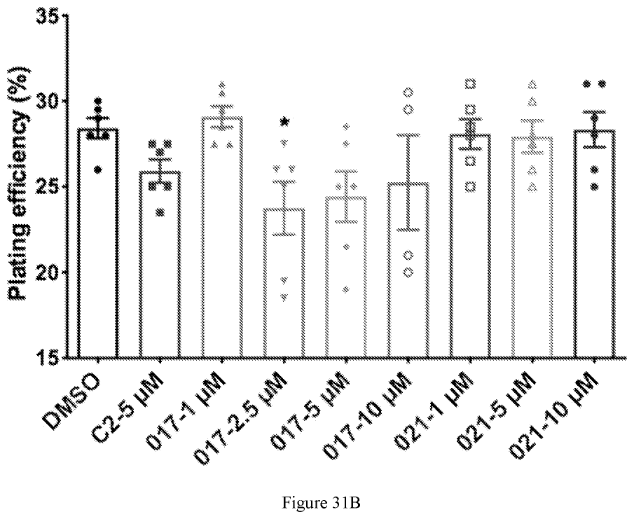

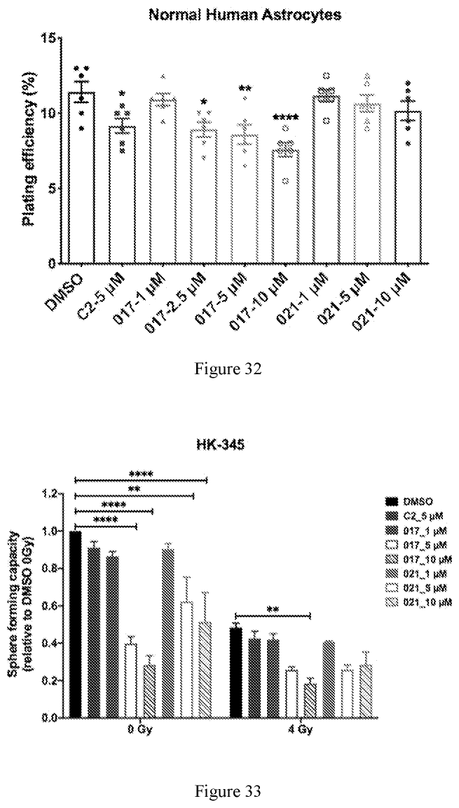

Cells were plated at clonal density into 6-well plated, treated and incubated for 10 days, fixed with methanol, and stained with crystal violet. Colonies of more than 50 cells were counted and expressed as percent of cells plated. Results are shown in FIGS. 28-32.

### Western Blotting

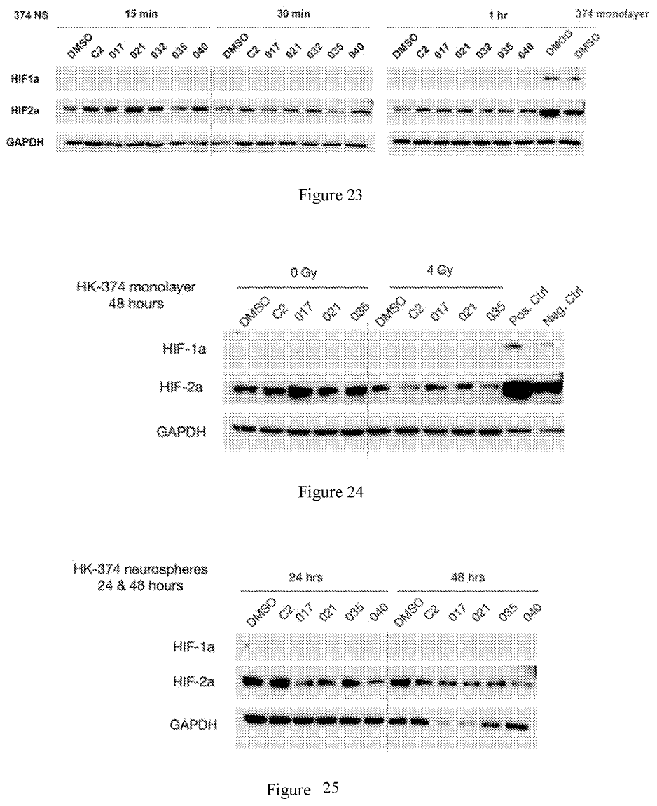

Patient-derived HK-374 spheres were dissociated and plated at the density of 1×10\hat{ }6 cells/well in the ultra-low adhesion 6-well plates. The next day, the cells were treated with 10 NM Compound 2 (C2) or MXC-017 or MXC-021 or MXC-032 or MXC-035 or MXC-040 or solvent control (DMSO), along with dimethyloxalylglycine (DMOG) as the positive control. 15 minutes, 30 minutes and 1 hour after the drug treatment, the spheres were collected and lysed in 100 μl of ice-cold RIPA lysis buffer (10 mM Tris-HCl (pH 8.0), 1 mM EDTA, 1% Triton X-100, 0.1% Sodium Deoxycholate, 0.1% SDS, 140 mM NaCl, 1 mM PMSF) containing proteinase inhibitor (Thermo Fisher Scientific) and phosphatase inhibitor (Thermo Fisher Scientific). The protein concentration in each sample was determined by BCA protein assay (Therma Fisher Scientific) and samples were denaturated in 4× Laemmli sample buffer (Bio-Rad) containing 10% 0-mercaptoethanol for 10 minutes at 95° C. Equal amounts of protein were loaded onto 10% SDS-PAGE gels (1× Stacking buffer—1.0 M Tris-HCl, 0.1% SDS, pH 6.8, 1× Separating buffer—1.5 M Tris-HCl, 0.4% SDS, pH 8.8) and were subjected to electrophoresis in 1× Running buffer (12.5 mM Tris-base, 100 mM Glycine, 0.05% SDS), initially at 40 V for 30 minutes followed by 80 V for two hours. Samples were then transferred onto 0.45 μM nitrocellulose membrane (Bio-Rad) for two hours at 80 V. Membranes were blocked in 1×TBST (20 mM Tris-base, 150 mM NaCl, 0.2% Tween-20) containing 5% milk or 5% bovine serum albumin (BSA) for 20 minutes and then washed with 1×TBST followed by incubation with primary antibodies against HIF1α (Cat #361695, 1:1000, Cell Signaling), HIF2a (Cat #7096S, 1:1000, Cell Signaling) and GAPDH (Cat #ab9484, 1:5000, Abcam) in 1×TBST containing 5% BSA overnight at 4° C. with gentle rocking. Membranes were then washed three times for 5 minutes each with 1×TBST and incubated with secondary antibodies, 1:1000 anti-rabbit or anti-mouse horseradish peroxidase (HRP; Cell Signaling) in 5% BSA/TBST for two hours at room temperature with gentle rocking. Membranes were washed again three times for 5 minutes each with 1×TBST Pierce ECL Plus Western Blotting Substrate (Thermo Fisher Scientific) was added to each membrane and incubated at room temperature for 5 minutes. The blots were then scanned with Odyssey Fc Imaging system. GAPDH was used as a loading control. Results are shown in FIGS. 23-25.

### RNA Sequencing (RNASeq)

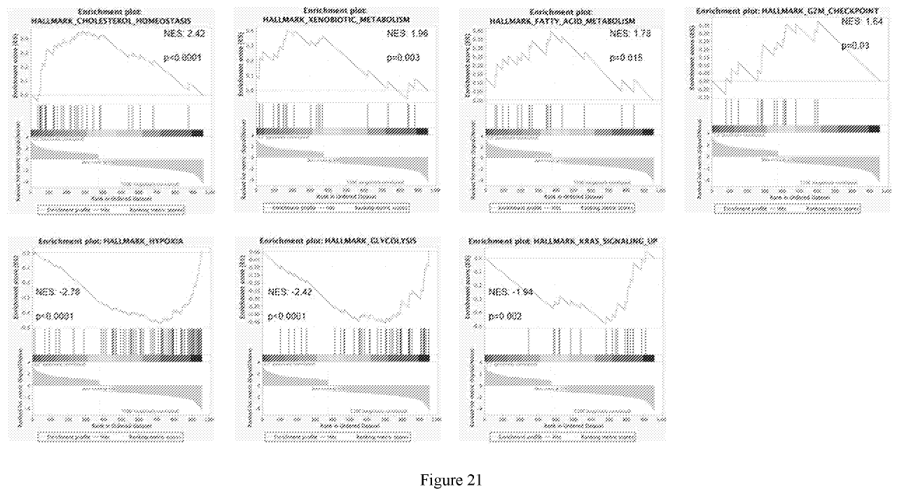

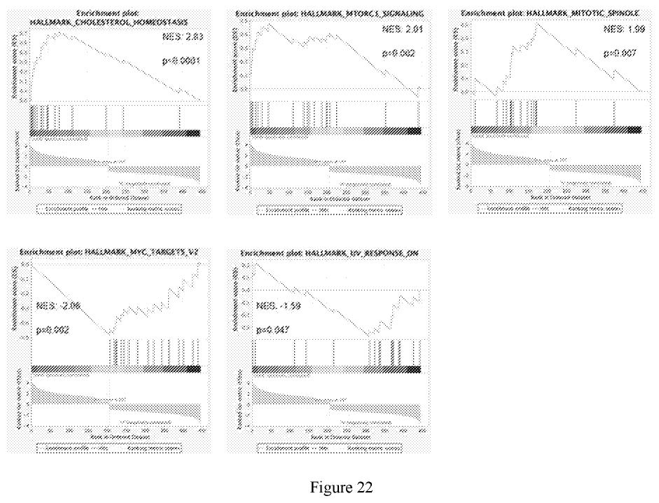

48 hours after 4 Gy irradiation or sham irradiation, RNA was extracted from HK-374 cells using Trizol. RNASeq analysis was performed by Novogene (Chula Vista, CA). Quality and integrity of total RNA was controlled on Agilent Technologies 2100 Bioanalyzer (Agilent Technologies; Waldbronn, Germany). The RNA sequencing library was generated using NEBNext® Ultra RNA Library Prep Kit (New England Biolabs) according to manufacturer's protocols. The library concentration was quantified using a Qubit 3.0 fluorometer (Life Technologies), and then diluted to 1 ng/pl before checking insert size on an Agilent Technologies 2100 Bioanalyzer (Agilent Technologies; Waldbronn, Germany) and quantifying to greater accuracy by quantitative Q-PCR (library molarity >2 nM). The library was sequenced on the Illumina NovaSeq6000 platform. Results are shown in FIGS. 21-22.

Downstream analysis was performed using a combination of programs including STAR, HTseq, and Cufflink. Alignments were parsed using the program Tophat and differential expressions were determined through DESeq2. Reference genome and gene model annotation files were downloaded from genome website browser (NCBI/UCSC/Ensembl) directly. Indexes of the reference genome were built using STAR and paired-end clean reads were aligned to the reference genome, using STAR (v2.5). HTSeq v0.6.1 was used to count the read numbers mapped of each gene. The FPKM of each gene was calculated based on the length of the gene and reads count mapped to this gene.

Differential expression analysis between irradiated and control samples (three biological replicates per condition) was performed using the DESeq2 R package (2_1.6.3). The resulting p-values were adjusted using the Benjamini and Hochberg's approach for controlling the False Discovery Rate (FDR). Genes with an adjusted p-value of <0.05 found by DESeq2 were assigned as differentially expressed. Gene set enrichment analysis was performed using the GSEA software, a joint project of UC San Diego and Broad Institute (Subramanian et al., PNAS, 2005, 102, 15545-50).

### In Vivo Analysis

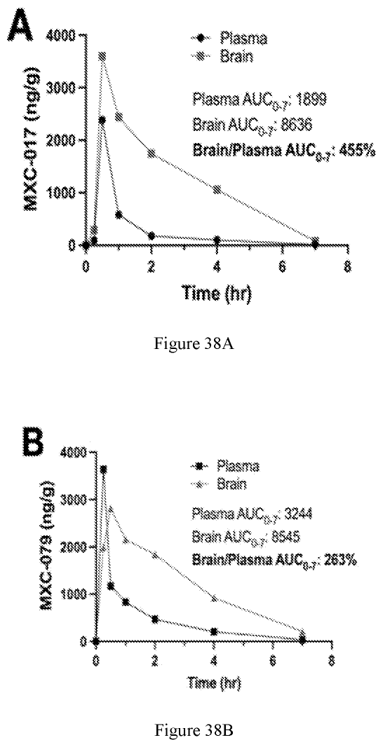

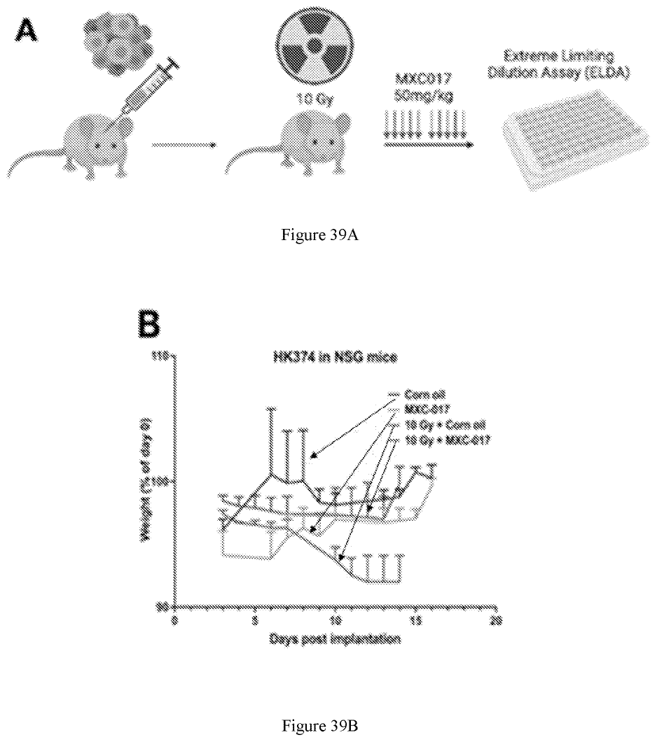

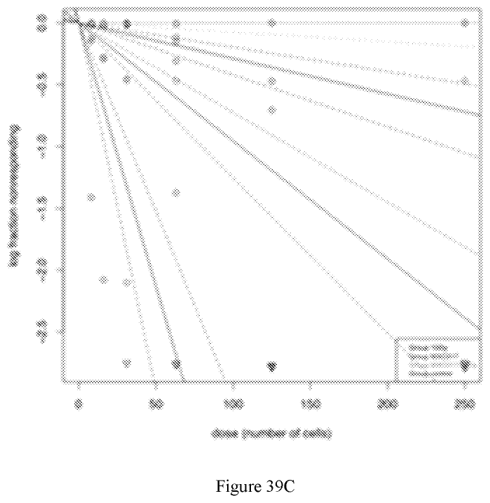

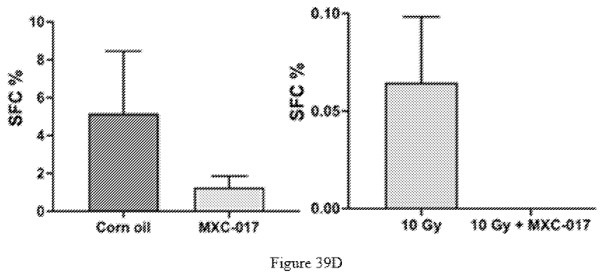

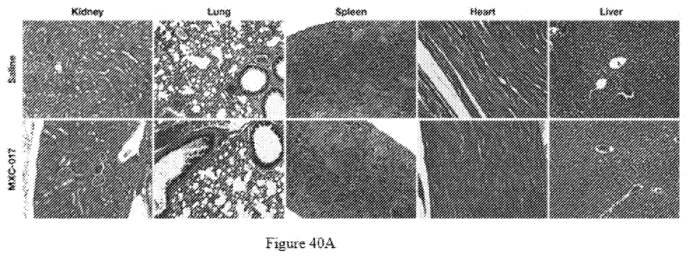

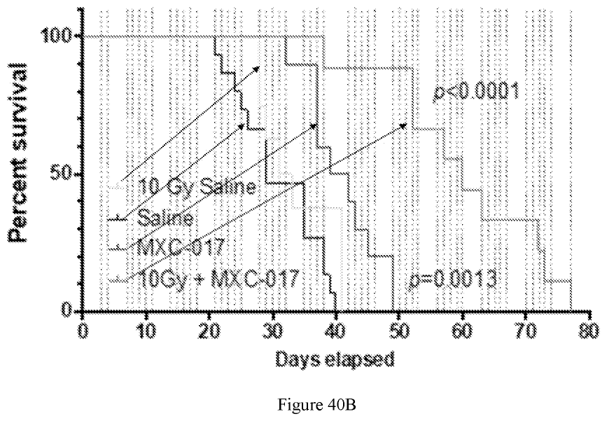

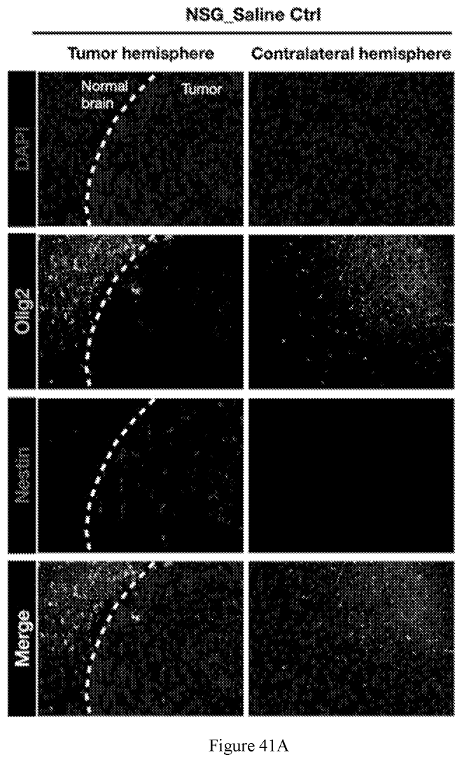

Data showing that MXC-017 and MXC-079 cross the BBB with brain to plasma ratios of 4.55 and 2.6 and peak brain concentrations in the micromolar range (FIGS. 38A and 38B). FIG. 39A shows the outline of an experiment, designed to test if MXC-017 targets glioma stem cells in vivo. MXC-017 was well tolerated in mice with no acute clinical toxicity. Compared to control mice or mice treated with radiation alone, MCX-017 treatment alone or in combination with radiation allowed animals to gain weight during the observation time of 2 weeks (FIG. 39B). After two weeks of treatment the tumors were harvested, processed into single cells and the number of glioma cells with sphere-forming capacity (glioma stem cells) was determined in a functional assay (extreme limiting dilution assay). Treatment of the mice with MXC-017 alone or in combination with radiation reduced the number of sphere forming cells (FIGS. 39C/D), and significantly reduced the frequency of glioma stem cells (FIGS. 39A-D). After treatment of the mice with MXC-017 at 50 mg/kg/day for two weeks, observe histological signs of toxicity in any of the major organs (kidneys, lung, spleen, heart, or liver) were not observed. (FIG. 40A).

Continuous treatment of the mice with five daily doses of MXC-017/week led to a small but significant increase in median survival. Combined treatment with radiation and MXC-017 significantly prolonged survival in PDOXs bearing mice from 29 to 60 days (FIG. 40B).

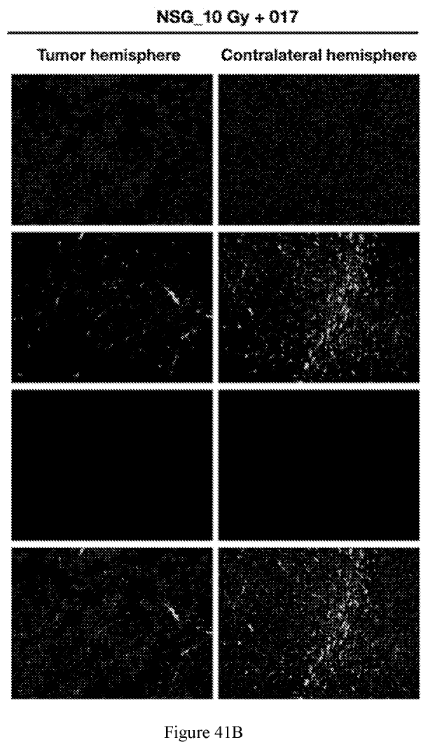

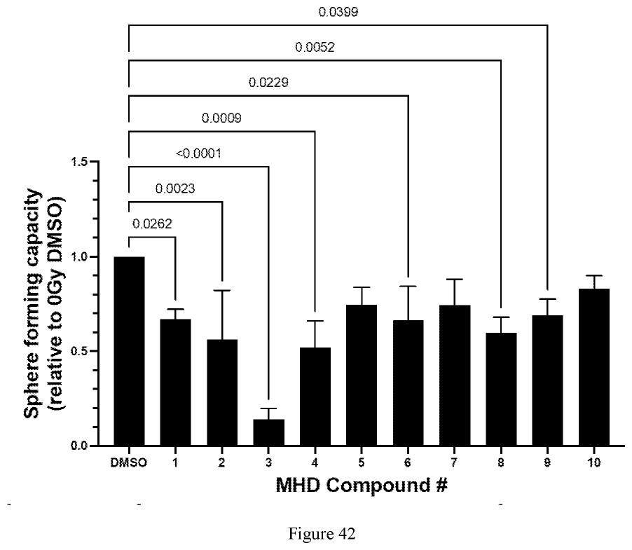

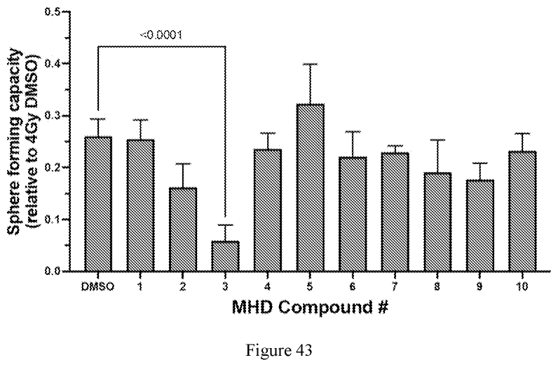

In line with the above observations of efficacy against glioma stem cells and lack of normal tissue toxicity, it was found that combined treatment with radiation and MXC-017 (FIG. 41B) eliminated Nestin-positive glioma stem cells in brain sections of glioma-bearing mice but had no effect on Olig1-positive oligodendrocytes, a sensitive normal tissue cell population in the brain (FIGS. 41A-B).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound represented by formula (I) or a pharmaceutically acceptable salt thereof: 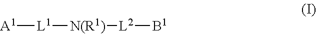
wherein
(1) L¹ is C₁-C₆ alkylene, C₂-C₆ alkenylene, or —C(O)—;
R¹ is H, or C₁-C₆ alkylene; and
A¹ is 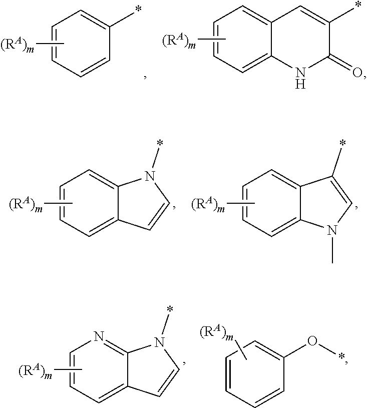
or 5- or 6-membered heteroaryl optionally substituted with one or more R^{A}, where * denotes the linkage to L¹;
or
(2) L¹ is —C(═CH₂)—, —C(R³)(R⁴)—, or —C(O)—; and
R¹ and A¹ together with the nitrogen to which R¹ and L¹ is attached combine to form: 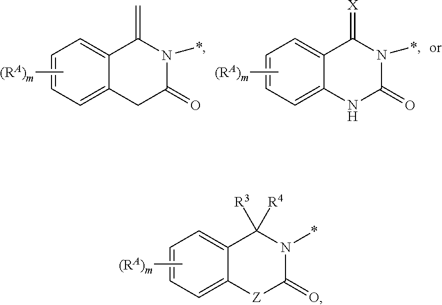
where * denotes the linkage to L²,
R³ and R⁴ are each independently H, —OH, C₁-C₆ alkyl, C₁-C₆ haloalkyl, or phenyl;
X is O or CH₂, and
Z is NH or CH₂;
each R^{A} is independently halo, —OH, —S(O)₂NR⁵R⁶, C₁-C₆ alkyl optionally substituted with amino, C₁-C₆ alkoxy, C₁-C₆ haloalkyl, —NR⁵R⁶, nitro, cyano, or —C(O)NR⁵R⁶;
L² is:
(1) C₁-C₆ alkylene, C₂-C₆ alkenylene, or —C(O)NH—, or
(2) L² is **=C(H)—* wherein * denote the linkage to A¹ and ** denotes the attachment to N and R¹ is a bond to L²;
B¹ is aryl optionally substituted with one or more R^{B} groups, 5- or 6-membered heteroaryl optionally substituted with one or more R^{B} groups, or NR⁵R⁶;
each R^{B} is independently halo, C₁-C₆ alkoxy, —C(O)C₁-C₆ alkyl, —S(O)₂NR⁵R⁶, or —C(O)NR⁵R⁶;
R⁵ and R⁶ are each independently H, alkyl, cycloalkyl, heterocyclyl, or phenyl, wherein alkyl, cycloalkyl, heterocyclyl, and phenyl is optionally substituted with R⁷, or
R⁵ and R⁶ taken together with the attached nitrogen form a heterocyclyl optionally substituted with R⁷;
R⁷ is —OH, halo, C₁-C₆ alkyl, C₁-C₆ alkylene-phenyl, or phenyl optionally substituted with halo;
m is 0, 1, 2, or 3;
and
n is 0, 1, 2, or 3.

2. The compound of claim 1, wherein L¹ is —C(═CH₂)—, —C(R³)(R⁴)—, or —C(O)—, and R¹ and A¹ together with the nitrogen to which R¹ and L¹ is attached combine to form

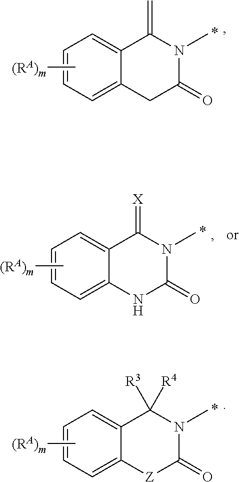

3. The compound of any one of the preceding claims, wherein Z is NH.

4. The compound of any one of claims 1-3, wherein Z is CH₂.

5. The compound of claim 1 or 2, wherein A¹ is

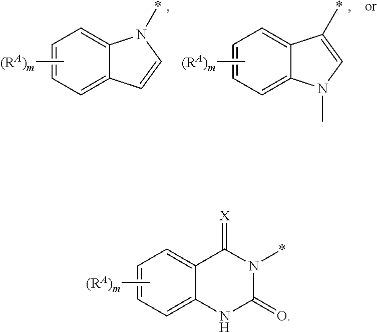

6. The compound of any one of the preceding claims, wherein B¹ is

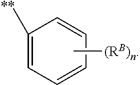

7. The compound of claim 1, claim 5, or claim 6, wherein the compound is of formula: 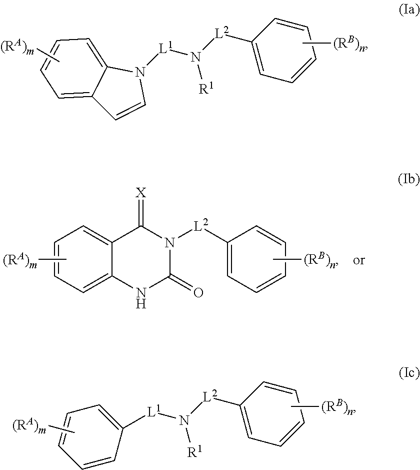
or a pharmaceutically acceptable salt thereof.

8. The compound of any one of the preceding claims, wherein X is O.

9. The compound of any of claims 1-7, wherein X is CH₂.

10. The compound of any one of the preceding claims, wherein m is O.

11. The compound of any one of claims 1-10, wherein R^{A}, when present, is halo.

12. The compound of any one of claims 1-11, wherein R^{A}, when present, is chloro.

13. The compound of any one of claims 1-11, wherein R^{A}, when present, is fluoro.

14. The compound of any one of claims 1-13, wherein R^{B} is —S(O)₂NR⁵R⁶.

15. The compound of any one of claim 1 or 5-14, wherein the compound is of formula: 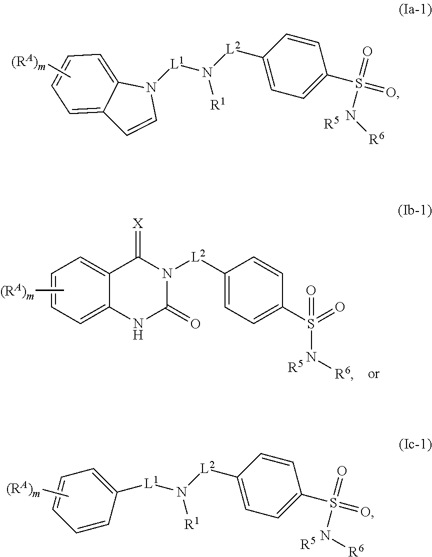
or a pharmaceutically acceptable salt thereof.

16. The compound of any one of the preceding claims, wherein L¹ is —C(O)—.

17. The compound of any one of the preceding claims, wherein L² is C₁-C₆ alkylene.

18. The compound of any one of the preceding claims, wherein L² is methylene.

19. The compound of any one of claim 1 or 5-18, wherein R¹ is H.

20. The compound of claim 1 or 5-19, wherein the compound is of formula: 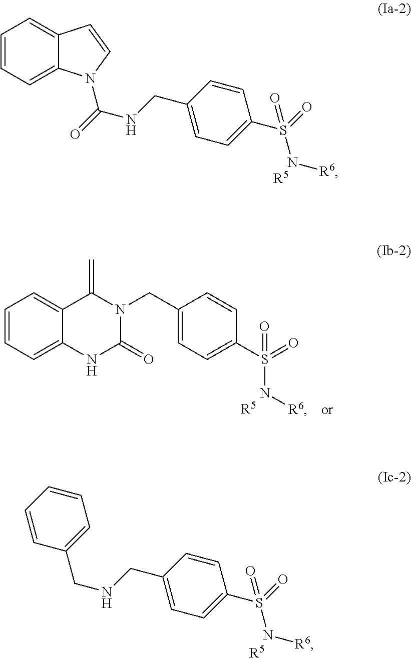
or a pharmaceutically acceptable salt thereof.

21. The compound of any one of the preceding claims, wherein R⁵ and R⁶ are each independently an alkyl optionally substituted with R⁷.

22. The compound of any one of the preceding claims, wherein R⁵ and R⁶ are each independently methyl or ethyl optionally substituted with R⁷.

23. The compound of any one of the preceding claims, wherein R⁷, when present, is phenyl or —OH.

24. The compound of any one of claims 1-23, wherein R⁵ and R⁶ taken together with the attached nitrogen form a heterocyclyl optionally substituted with R⁷.

25. The compound of claim 24, wherein each R^{B} is

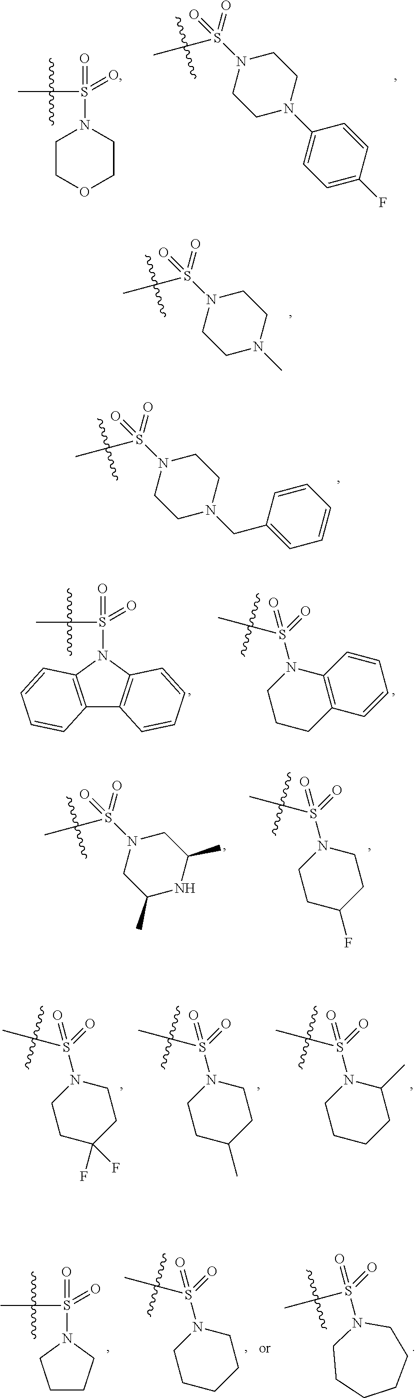

26. The compound of claim 24, wherein R⁵ and R⁶ taken together with the attached nitrogen form an unsubstituted heterocyclyl.

27. The compound of claim 26, wherein each R^{B} is

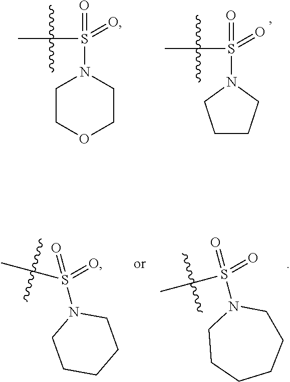

28. The compound of claim 1, or claim 15, wherein the compound is of formula: 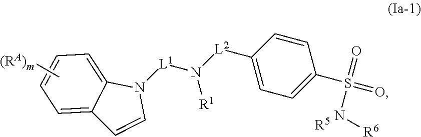
or a pharmaceutically acceptable salt thereof,
wherein L¹ is —C(O)—;
R¹ is H, or C₁-C₆ alkylene;
each R^{A} is independently halo, C₁-C₆ alkyl optionally substituted with amino, C₁-C₆ alkoxy, C₁-C₆ haloalkyl, nitro, cyano, or —C(O)NR⁵R⁶;
L² is:
C₁-C₆ alkylene;
R⁵ and R⁶ are each independently H, or R⁵ and R⁶ taken together with the attached nitrogen form a heterocyclyl optionally substituted with R⁷;
R⁷ is —OH, halo, C₁-C₆ alkyl, C₁-C₆ alkylene-phenyl, or phenyl optionally substituted with halo; and
m is 0, 1, or 2.

29. A compound selected from: 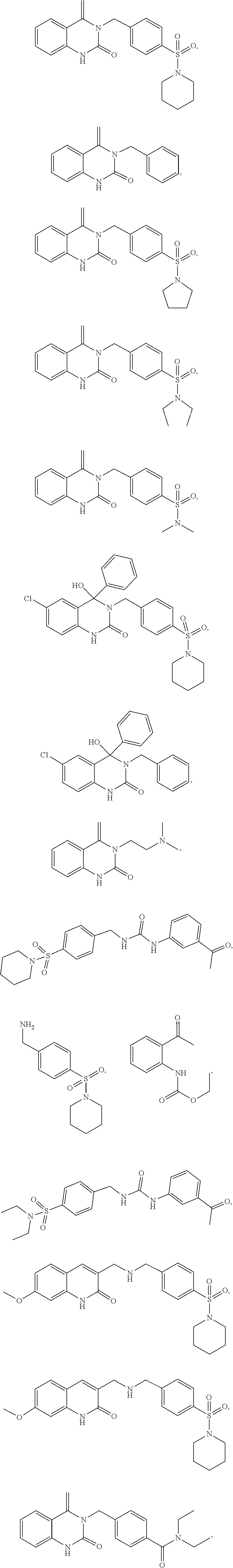 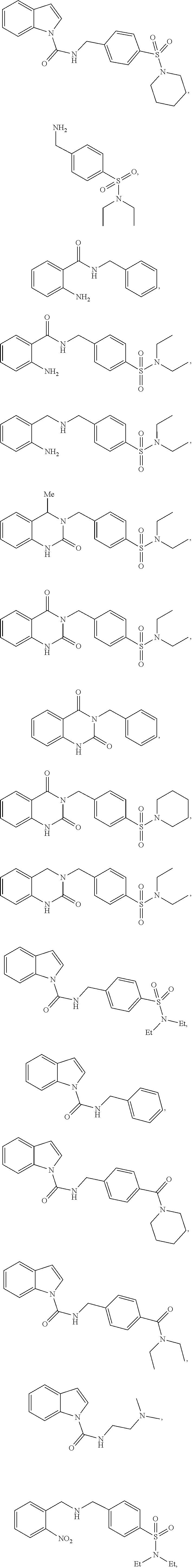 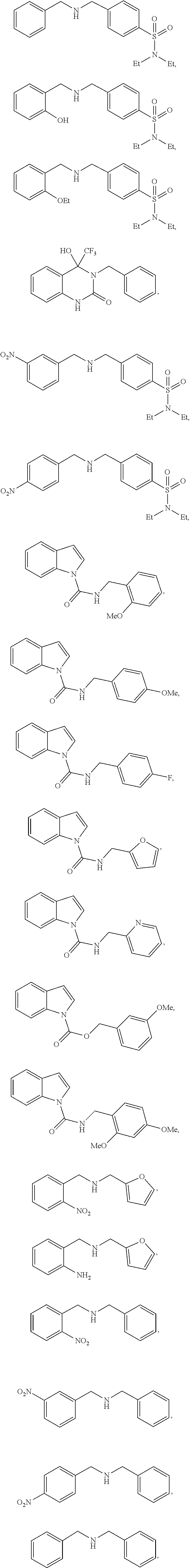 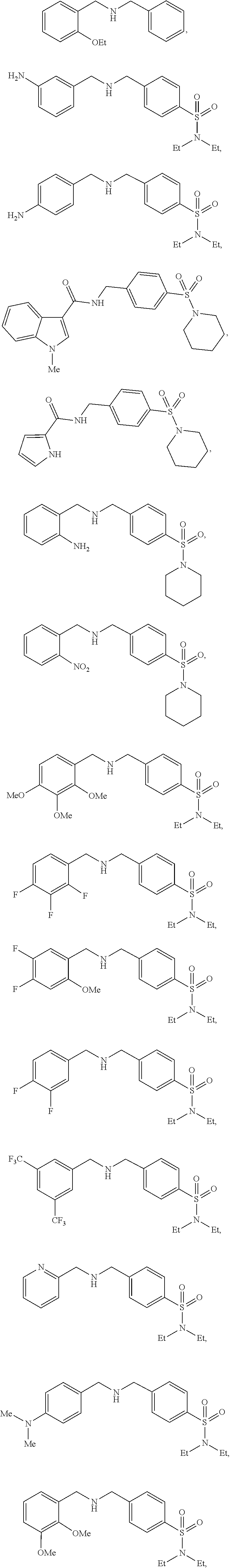 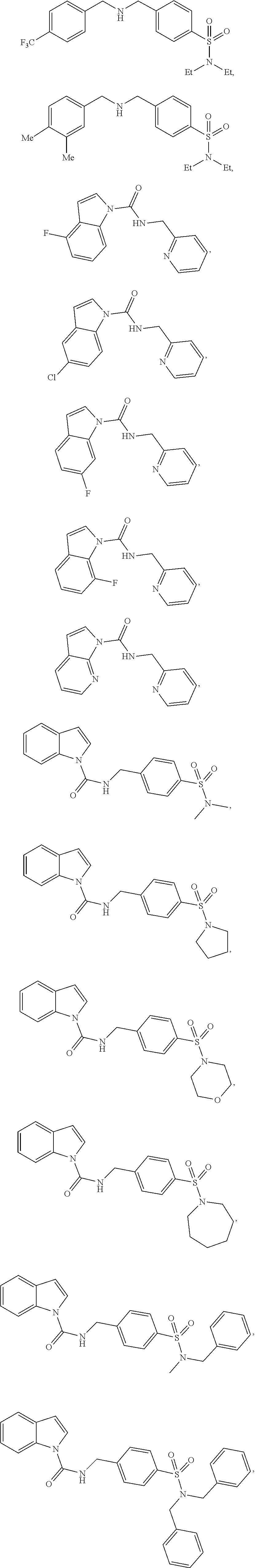 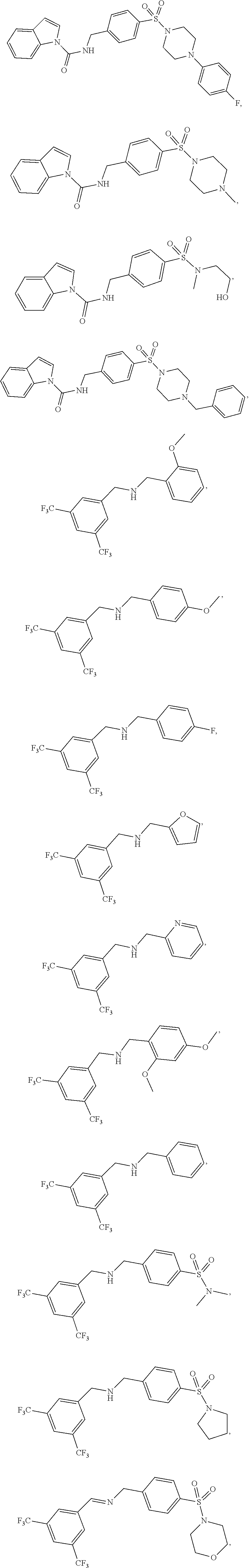 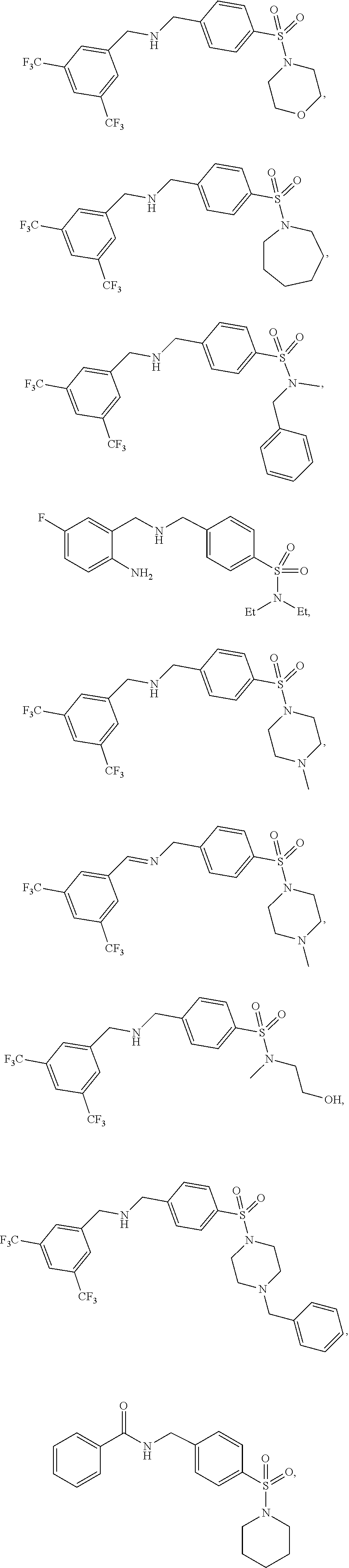 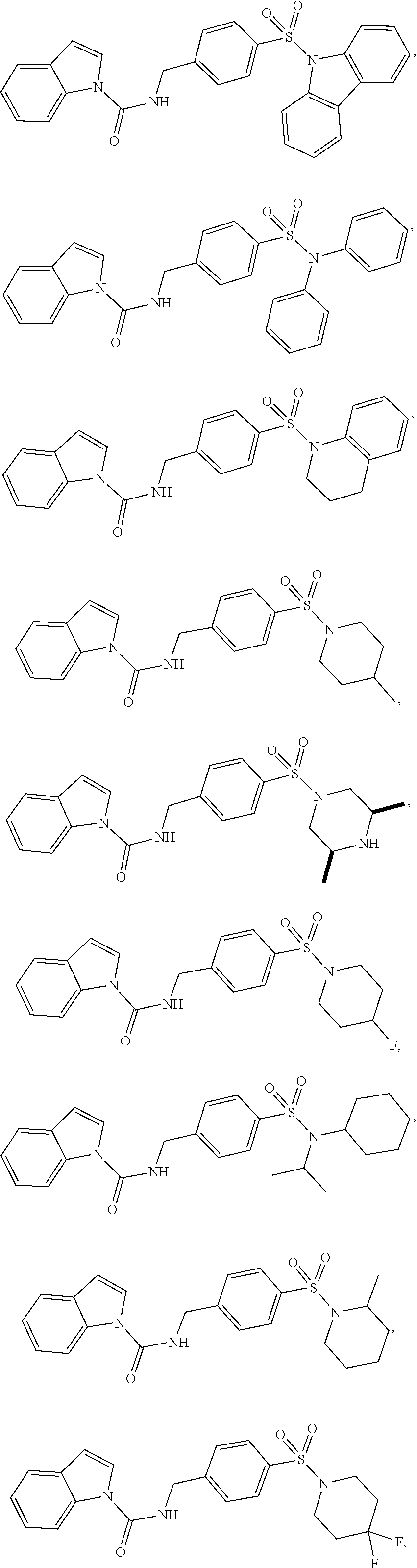 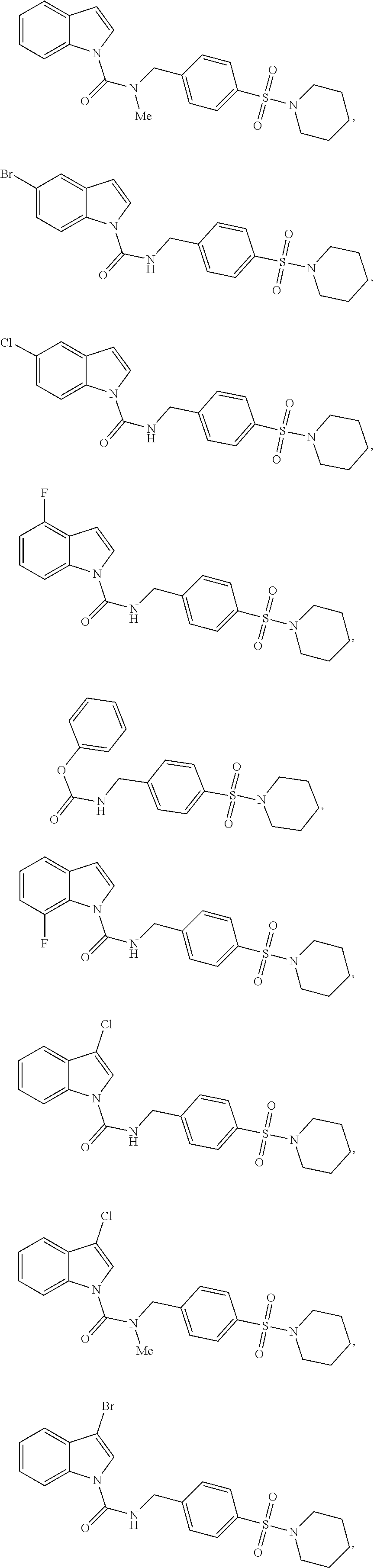 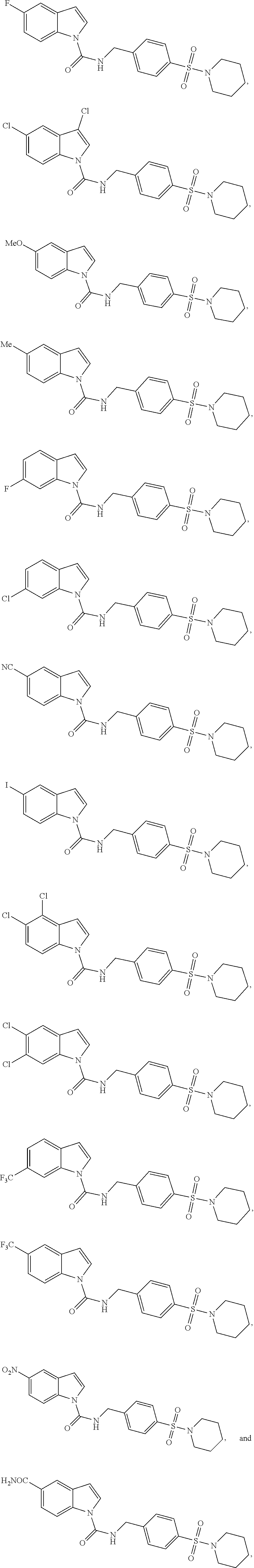
or a pharmaceutically acceptable salt thereof.

30. A pharmaceutical composition comprising a compound of any one of the preceding claims and a pharmaceutically acceptable excipient.

31. A method of treating cancer in a subject in need thereof, comprising administering to the subject a compound of any one of claims 1-29 or a composition of claim 30.

32. The method of claim 31, wherein the subject in need thereof has been treated with radiation.

33. The method of any one of claims 31-32, wherein the cancer is glioblastoma or breast cancer.

34. A method of preventing conversion of non-stem cancer cells into induced cancer-initiating cells, comprising administering to the subject a compound of any one of claims 1-29 or a composition of claim 30.

35. The method of claim 34, wherein the non-stem cancer cells have been treated with radiation.

36. The method of claim 34 or claim 35, wherein the non-stem cancer cells are glioblastoma cells.
